(19) United States
(12) Patent Application Publication
(10) Pub. No.: US 2005/0004177 A1
(43) Pub. Date: Jan. 6, 2005

(54) Combination of an allosteric inhibitor of matrix metalloproteinase-13 and a ligand to an alpha-2-delta receptor

(75) Inventor: William Howard Roark, Ann Arbor, MI (US)

Correspondence Address: WARNER-LAMBERT COMPANY, 2800 PLYMOUTH RD, ANN ARBOR, MI, 48105, US

(73) Assignee: WARNER-LAMBERT COMPANY LLC

(21) Appl. No.: 10/883,899

(22) Filed: Jul. 2, 2004

Related U.S. Application Data
(60) Provisional application No. 60/484,577, filed on Jul. 2, 2003.

(51) Int. Cl. A61K 31/4439; A61K 31/41

(52) U.S. Cl. 514/341; 514/381

(57) ABSTRACT

This invention relates to a combination of an allosteric inhibitor of matrix metalloproteinase-13, or a pharmaceutically acceptable salt thereof, and a ligand to an alpha-2-delta receptor, or a pharmaceutically acceptable salt thereof, a pharmaceutical composition comprising the combination, and a method of using the combination to treat a disease or disorder in a mammal suffering therefrom, wherein the disease or disorder is responsive to treatment in one aspect by an allosteric inhibitor of MMP-13 and in the same or a different aspect by a ligand to an alpha-2-delta receptor.

### CROSS-REFERENCE TO RELATED APPLICATIONS This application claims benefit of priority from U.S. Provisional Patent Application No. 60/484,577 filed Jul. 2, 2003.

### FIELD OF THE INVENTION

This invention relates to a combination of an allosteric inhibitor of matrix metalloproteinase-13 and a ligand to an alpha-2-delta receptor, a pharmaceutical composition comprising the combination, and a method of using the combination to treat a disease or disorder in a mammal suffering therefrom, wherein the disease or disorder is responsive to treatment in one aspect by an allosteric inhibitor of MMP-13 and in the same or a different aspect by a ligand to an alpha-2-delta receptor.

### BACKGROUND OF THE INVENTION

Alpha-2-delta receptor ligands, including gabapentin and pregabalin, have been found to be effective for treating diseases and disorders such as epilepsy, convulsions, neuropathic pain, including post-herpetic neuralgia, fibromyalgia, including symptoms of fibromyalgic pain, sleep disturbance, and fatigue, social anxiety disorder, generalized anxiety disorder, panic disorder, migraine, hot flashes, restless legs syndrome, osteoarthritis, osteoarthritic pain, inflammation, inflammatory pain, including rheumatoid arthritic pain, and rheumatoid arthritis, and for inhibiting cartilage damage in a joint.

Matrix metalloproteinases (sometimes referred to as MMPs) comprise a family of more than twenty naturally occurring enzymes most of which are found in most mammals. Over-expression and activation of MMPs or some other pathological imbalance between MMPs and their naturally occurring inhibitors, the tissue inhibitors of metalloproteinases (“TIMPs”), has been causally linked to the pathogenesis of diseases characterized by the breakdown of extracellular matrix or connective tissues. Pathological imbalance or over-expression and activation of MMP-13 has been directly implicated in diseases such as, for example, osteoarthritis, rheumatoid arthritis, cartilage damage, abdominal aortic aneurysms, skin ulcers, and metastasis of a cancer selected from: ovarian cancer, squamous carcinoma, head carcinoma, neck carcinoma, fibrosarcoma, chondrosarcoma, basal cell carcinoma of the skin, and breast cancer.

Applicant's invention is a combination of an allosteric inhibitor of MMP-13, or a pharmaceutically acceptable salt thereof, and an alpha-2-delta receptor ligand, or a pharmaceutically acceptable salt thereof, that is particularly useful for treating a disease or disorder responsive to treatment in one aspect by an allosteric inhibitor of MMP-13 and in the same or a different aspect by a ligand to an alpha-2-delta receptor. All that is required to treat such diseases or disorders in a mammal suffering therefrom according to a method of the present invention is to administer to the mammal a therapeutically effective, sufficiently nontoxic amount of a combination or pharmaceutical composition of the present invention.

### SUMMARY OF THE INVENTION

There are many aspects of the present invention. One aspect of this invention is a combination, comprising an allosteric inhibitor of MMP-13, or a pharmaceutically acceptable salt thereof, and a ligand to an alpha-2-delta receptor, or a pharmaceutically acceptable salt thereof.

Another aspect of this invention provides a pharmaceutical composition, comprising a combination of an allosteric inhibitor of MMP-13, or a pharmaceutically acceptable salt thereof, a ligand to an alpha-2-delta receptor, or a pharmaceutically acceptable salt thereof, and a pharmaceutically acceptable carrier, diluent, or excipient.

Still another aspect of this invention is a method of treating a disease or disorder that is responsive to treatment by a combination of an allosteric inhibitor of MMP-13, or a pharmaceutically acceptable salt thereof, and an alpha-2-delta receptor ligand, or a pharmaceutically acceptable salt thereof, in a mammal suffering therefrom, comprising administering to the mammal a therapeutically effective amount of a pharmaceutical composition comprising said combination, and a pharmaceutically acceptable carrier, diluent, or excipient.

Still another aspect of this invention is a combination, pharmaceutical composition, or method of this invention, wherein the allosteric inhibitor of MMP-13, or a pharmaceutically acceptable salt thereof, is a allosteric inhibitor of MMP-13 named in any one of the below listed U.S. patents, U.S. patent applications, U.S. patent application publications, European patents, European patent application publications, or PCT International Patent Application Publications, or a pharmaceutically acceptable salt thereof.

Still another aspect of this invention is a combination, pharmaceutical composition, or method of this invention, wherein the allosteric inhibitor of MMP-13 is a compound of Formula IA 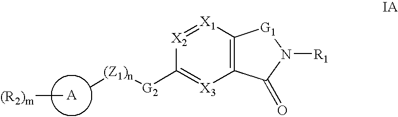
wherein:

X₁, X₂, and X₃, independently of each other, represent a nitrogen atom or a group -CR₃ in which R₃ represents a group selected from hydrogen, (C₁-C₆)alkyl, amino, mono(C₁-C₆)alkylamino, di(C₁-C₆)alkylamino, hydroxy, (C₁-C₆)alkoxy, and halogen,

with the proviso that not more than two of the groups X₁, X₂ and X₃ simultaneously represent a nitrogen atom,

G₁ represents a group selected from those of formulae (i/a) and (i/b): 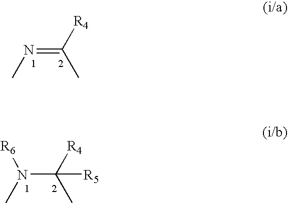
in which:

the carbon atom with number 2 is attached to the group N-R₁, in the ring,

R₄ and R₅, identical or different, independently of each other, represent a group selected from hydrogen, (C₁-C₆)alkyl, aryl, aryl(C₁-C₆)alkyl, cycloalkyl, cycloalkyl(C₁-C₆)alkyl, heteroaryl, heteroaryl(C₁-C₆)alkyl, heterocycloalkyl, and heterocycloalkyl(C₁-C₆)alkyl,

R₆ represents a group selected from:

hydrogen, trifluoromethyl, OR₇, NR₇R₈, in which R₇ and R₈, identical or different independently of each other, represent hydrogen or (C₁-C₆)alkyl,

(C₁-C₆)alkyl, (C₂-C₆)alkenyl, (C₂-C₆)alkynyl, aryl, aryl(C₁-C₆)alkyl, cycloalkyl(C₁-C₆)alkyl, heteroaryl, heteroaryl(C₁-C₆)alkyl, heterocycloalkyl, and heterocycloalkyl(C₁-C₆)alkyl, these groups being optionally substituted by one or more groups, which may be identical or different independently of each other, selected from halogen, amino, mono(C₁-C₆)alkylamino, di(C₁-C₆)alkylamino, each alkyl moiety being identical or different independently of each other, cyano, trihalogeno(C₁-C₆)alkyl, (C₁-C₆)acyl, —C(=O)OR₇, —OR₇ and —SR₇, in which R₇ is as defined hereinbefore,

G₂ represents a group selected from carbon-carbon triple bond, -CH=C=CH-, C=O, C=S, S(O)ₙ₁ in which n1 represents an integer from 0 to 2 inclusive, and a group of formula (i/c): 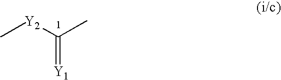
in which the carbon atom with number 1 is attached to the bicycle of the compound of formula (I), Y₁ represents a group selected from oxygen, sulphur, —NH and —N(C₁-C₆)alkyl, and Y₂ represents a group selected from oxygen, sulphur, —NH and —N(C₁-C₆)alkyl,

n represents an integer from 0 to 6 inclusive,

Z₁ represents —CR₉R₁₀, wherein R₉ and R₁₀, identical or different independently of each other, represent a group selected from hydrogen, (C₁-C₆)alkyl, trihalogeno(C₁-C₆)alkyl, halogen, —OR₇, —SR₇, and -C(=O)OR₇, in which R₇ is as defined hereinbefore, amino, mono(C₁-C₆)alkylamino, di(C₁-C₆)alkylamino in which each alkyl moiety is identical or different independently of each other, and

wherein when n is greater than or equal to 2, the hydrocarbon chain Z₁, optionally contains one to two isolated or conjugated multiple bonds,

and/or wherein when n is greater than or equal to 2, one of said —CR₉R₁₀ may optionally be replaced with a group selected from oxygen, S(O)ₙ₁ in which n1 is as defined hereinbefore, —NH and —NH(C₁-C₆)alkyl,

A represents a group selected from aryl, heteroaryl, cycloalkyl, and heterocycloalkyl, these groups being 5- or 6-menbered monocycle or bicycle composed of two 5- or 6- membered monocycle,

R₁ represents a group selected from:

hydrogen,

(C₁-C₆)alkyl, (C₂-C₆)alkenyl, (C₂-C₆)alkynyl, these groups may be optionally substituted with one or more groups, which may be identical or different independently of each other, selected from amino, cyano, trihalogeno(C₁-C₆)alkyl, cycloalkyl, —C(=O)NR₇R₈, —C(=O)OR₈, OR₈, SR₈, in which R₇ and R₈, which may be identical or different independently of each other, represent hydrogen or (C₁-C₆)alkyl,

and the group of formula (i/d):

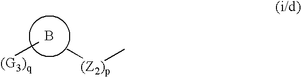

in which p is an integer from 0 to 8 inclusive,

Z₂ represents —CR₁₁R₁₂ wherein R₁₁ and R₁₂, identical or different independently of each other, represent a group selected from hydrogen, (C₁-C₆)alkyl, phenyl, trihalogeno(C₁-C₆)alkyl, halogen, amino, OR₇, SR₇ and —C(=O)OR₇ in which R₇ represents hydrogen or (C₁-C₆)alkyl, and

wherein when p is greater than or equal to 2, the hydrocarbon chain Z₂ optionally contains one or two isolated or conjugated multiple bonds,

and/or wherein n is greater than or equal to 2, one of said —CR₁₁R₁₂ may optionally be replaced with a group selected from oxygen, S(O)ₙ₁ in which n1 is as defined hereinbefore, —NH, —NH(C₁-C₆)alkyl, and carbonyl,

B represents a group selected from aryl, heteroaryl, cycloalkyl, and heterocycloalkyl, these groups being 5- or 6-menbered monocycle or bicycle composed of two 5- or 6- membered monocycle,

q is an integer from 0 to 7 inclusive,

the group(s) G₃, which may be identical or different independently of each other, is (are) selected from (C₁-C₆)alkyl, halogen, CN, NO₂, CF₃, OCF₃, —(CH₂)ₖNR₁₃R₁₄, —N(R₁₃)C(=O)R₁₄, —N(R₁₃)C(=O)OR₁₄, —N(R₁₃)SO₂R₁₄, —N(SO₂R₁₃)₂, —OR₁₃, —S(O)ₖ₁R₁₃, —SO₂—N(R₁₃) —(CH₂)ₖ₂-NR₁₄R₁₅, —(CH₂)ₖSO₂NR₁₃R₁₄, X₄(CH₂)ₖC(=O)OR₁₃, —(CH₂)ₖC(=O)OR₁₃, —C(=O)O—(CH₂)ₖ₂—NR₁₃R₁₄, —C(=O)O—(CH₂)ₖ₂—C(=O)OR₁₆, —X₄(CH₂)ₖC(=O)NR₁₃R₁₄, (CH₂)ₖC(=O)NR₁₃R₁₄, —R₁₇—C(=O)OR₁₃, —X₅—R₁₈, and —C(=O)—R₁₉—NR₁₃R₁₄ in which:

X₄ represents a group selected from oxygen atom, sulphur atom optionally substituted by one or two oxygen atoms, and nitrogen atom substituted by a hydrogen atom or a (C₁-C₆)alkyl group,

k is an integer from 0 to 3 inclusive,

k1 is an integer from 0 to 2 inclusive,

k₂ is an integer from 1 to 4 inclusive,

R₁₃, R₁₄ and R₁₅, which may be identical or different independently of each other, are selected from hydrogen and (C₁-C₆)alkyl,

R₁₆ represents a group selected from (C₁-C₆)alkyl, —R₁₉—NR₁₃R₁₄, —R₁₉—NR₁₃—C(=O)—R₁₉—NR₁₄R₁₅, and —C(=O)O—R₁₉—NR₁₃R₁₄ in which R₁₉ represents a linear or branched (C₁-C₆)alkylene group, and R₁₃, R₁₄ and R₁₅ are as defined hereinbefore,

R₁₇ represents a (C₃-C₆)cycloalkyl group,

X₅ represents a group selected from single bond, —CH₂—, oxygen atom, sulphur atom optionally substituted by one or two oxygen atoms, and nitrogen atom substituted by hydrogen atom or (C₁-C₆)alkyl group,

R₁₈ represents a group selected from:

5- or 6-menbered monocycle aryl, heteroaryl, which is optionally substituted by one or more groups, which may be identical or different, selected from (C₁-C₆)alkyl, halogen, hydroxy, cyano, tetrazolyl, amino, and —C(=O)OR₇ wherein R₇ represents hydrogen or (C₁-C₆)alkyl,

and 5- or 6-menbered monocycle cycloalkyl, heterocycloalkyl, which is optionally substituted by one or more groups, which may be identical or different, selected from (Cl-C₆)alkyl, halogen, hydroxy, oxo, cyano, tetrazolyl, amino, and —C(=O)OR₇ wherein R₇ represents hydrogen or (C₁-C₆)alkyl,

m is an integer from 0 to 7 inclusive,

the group(s) R₂, which may be identical or different independently of each other, is (are) selected from (C₁-C₆)alkyl, halogen, —CN, NO₂, SCF₃, —CF₃, —OCF₃, —NR₇R₈, —OR₈, —SR₈, —SOR₈, —SO₂R₈, —(CH₂)ₖSO₂NR₇R₈, —X₇(CH₂)ₖC(=O)OR₈, —(CH₂)ₖC(=O)OR₈, —X₇(CH₂)ₖC(=O)NR₇R₈, —(CH₂)ₖC(=O)NR₇R₈, and —X₈-R₂₀ in which:

X₇ represents a group selected from oxygen, sulphur optionally substituted by one or two oxygen atoms, and nitrogen substituted by hydrogen or (C₁-C₆)alkyl,

k is an integer from 0 to 3 inclusive,

R₇ and R₈, which may be identical or different independently of each other, are selected from hydrogen and (Cl-C₆)alkyl,

X₈ represents a group selected from single bond, —CH₂—, oxygen atom, sulphur atom optionally substituted by one or two oxygen atoms, and nitrogen atom substituted by hydrogen atom or (C₁-C₆)alkyl group,

R₂₀ represents 5- or 6-menbered monocycle aryl, heteroaryl, cycloalkyl, or heterocycloalkyl which is optionally substituted by one or more groups, which may be identical or different, selected from (C₁-C₆)alkyl, halogen, hydroxy and amino, and when the ring is heterocyclic, it comprises from 1 to 4 heteroatoms selected from nitrogen, oxygen and sulphur,

optionally, the racemic forms thereof, isomers thereof, N-oxides thereof, and the pharmaceutically acceptable salts thereof,

it being understood that when no specification are described:

an aryl group denotes an aromatic monocyclic or bicyclic system containing from 5 to 10 carbon atoms, and in the case of a bicyclic system, one of the ring of which is aromatic in character, and the other ring of which may be aromatic or partially hydrogenated,

a heteroaryl group denotes an aryl group as described above in which 1 to 4 carbon atoms are replaced by 1 to 4 hetero atoms selected from oxygen, sulfur and nitrogen,

a cycloalkyl group denotes a monocyclic or bicyclic system containing from 3 to 10 carbon atoms, this system being saturated or partially unsaturated but without aromatic character,

and a heterocycloalkyl group denotes a cycloalkyl group as defined hereinbefore in which 1 to 4 carbon atoms are replaced by 1 to 4 heteroatoms selected from oxygen, sulfur, and nitrogen.

Still another aspect of this invention is a combination, pharmaceutical composition, or method of this invention, wherein the allosteric inhibitor of MMP-13 is a compound of Formula IA, or a pharmaceutically acceptable salt thereof, wherein:

(i) G₂ represents a group selected from C=O, C=S, S(O)ₙ₁ in which n1 represents an integer from 0 to 2 inclusive, or a group of formula (i/c): 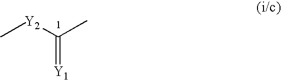
in which the carbon atom with number 1 is attached to the bicycle of the compound of formula (I), Y₁ represents a group selected from oxygen, sulphur, —NH and —NH(C₁-C₆)alkyl, and Y₂ represents a group selected from oxygen, sulphur, —NH and —NH(C₁-C₆)alkyl,

X₁, X₂, X₃, G₁ , n, Z₁, A, R₁, m and R₂ are as defined in formula (I), optionally, the racemic forms thereof, isomers thereof, N-oxides thereof, and the pharmaceutically acceptable salts thereof; or

(ii) G₂ represents a carbon-carbon triple bond,

n represents an integer from 1 to 6 inclusive,

X₁, X₂, X₃, G₁, Z₁, A, R₁, m and R₂ are as defined in formula (I), optionally, the racemic forms thereof, isomers thereof, N-oxides thereof, and the pharmaceutically acceptable salts thereof.

Still another aspect of this invention is a combination, pharmaceutical composition, or method of this invention, wherein the allosteric inhibitor of MMP-13 is a compound of Formula IA, or a pharmaceutically acceptable salt thereof, selected from:

(i) 3-(4-methoxy-benzyl)-4-oxo-3,4-dihydro-quinazoline-6-carboxylic acid 4-methoxy-benzylamide;

3-(4-methoxy-benzyl)-2-methyl-4-oxo-3,4-dihydro-quinazoline-6-carboxylic acid 4-methoxy-benzylamide, hydrochloride;

3-(4-methoxy-benzyl)-1-methyl-4-oxo-1,2,3,4-tetrahydro-quinazoline-6-carboxylic acid 4-methoxy-benzylamide;

3-(4-methoxy-benzyl)-1,2,2-trimethyl-4-oxo- 1,2,3 ,4-tetrahydro-quinazoline-6-carboxylic acid 4-methoxy-benzylamide;

4-[6-(4-methoxy-benzylcarbamoyl)-4-oxo-1,4-dihydro-2H-quinazolin-3-ylmethyl]-benzoic acid;

4-[6-(4-methoxy-benzylcarbamoyl)-1-methyl-4-oxo-1 ,4-dihydro-2H-quinazolin-3-ylmethyl]-benzoic acid methyl ester;

4-[6-(4-methoxy-benzylcarbamoyl)-1-methyl-4-oxo-1 ,4-dihydro-2H-quinazolin-3-ylmethyl]-benzoic acid;

3-(4-fluoro-benzyl)4-oxo-3,4-dihydro-quinazoline-6-carboxylic acid 3-methoxy-benzylamide;

3-(4-methanesulfonyl-benzyl-4-oxo-3 ,4-dihydro-quinazoline-6-carboxylic acid 4-methoxy-benzylamide;

4-Oxo-3-[4-(pyrrolidine- 1 -sulfonyl)-benzyl]-3,4-dihydro-quinazoline-6-carboxylic acid 4-methoxy-benzylamide;

4-[6-(3-methoxy-benzylcarbamoyl)-4-oxo-4H-quinazolin-3-ylmethyl]-benzoic acid;

3-(4-fluoro-benzyl)-4-oxo-3,4-dihydro-quinazoline-6-carboxylic acid (2-methoxy-pyridin-4-ylmethyl)-amide;

3-(3-fluoro-benzyl)-4-oxo-3,4-dihydro-pyrido[3,4-d]pyrimidine-6-carboxylic acid 3-methoxy-benzylamide;

3-(3-fluoro-benzyl)-4-oxo-3,4-dihydro-pyrido[3,4-d]pyrimidine-6-carboxylic acid 4-methoxy-benzylamide;

3-(3,4-Difluoro-benzyl)-4-oxo-3,4-dihydro-quinazoline-6-carboxylic acid (2-methoxy-pyridin-4-ylmethyl)-amide; and

3-(3,4-Difluoro-benzyl)-4-oxo-3,4-dihydro-quinazoline-6-carboxylic acid 4-methoxy-benzylamide, or a pharmaceutically acceptable salt thereof; or

(ii) 3-(4-fluorobenzyl)-6-(3-phenyl-pro-1-ynyl)-3H-quinazolin-4-one; methyl 4-[4-oxo-6-(3-phenyl-prop-1-ynyl)-4H-quinazolin-3-ylmethyl]-benzoate;

4-[4-oxo-6-(3-phenyl-prop-1-ynyl)4H-quinazolin-3-ylmethyl]-benzoic acid;

3-(4-fluorobenzyl)-6-(3-phenyl-prop-1-ynyl)-3H-pyrido[3,4-d]pyrimidin-4-one;

methyl 4-[6-(3-phenyl-prop-1-ynyl)-4-oxo-4H-pyrido[3,4-d]pyrimidin-3-ylmethyl]-benzoate;

4-[6-(3-phenyl-prop-1 -ynyl)-4-oxo-4H-pyrido[3,4-d]pyrimidin-3-ylmethyl]-benzoic acid;

4-[4-oxo-6-(3-phenyl-prop-1-ynyl)-4H-quinazoline-3-ylmethyl]-benzoic acid;

4-{6-[3-(4-methoxy-phenyl)-prop-1-ynyl]-4-oxo-4H-quinazoline-3-ylmethyl}-benzoic acid;

4-[4-oxo-6-(3-phenyl-prop-1-ynyl)-4H-quinazoline-3-ylmethyl]-benzamide;

3-[(3,5-difluoro-4-hydroxy)-benzyl]-6-(3-phenyl-prop-1-ynyl)-3H-quinazolin-4-one;

4-[6-(3-Imidazol-1-yl-prop-1-ynyl)-4-oxo-4H-quinazolin-3-ylmethyl]-benzoic acid;

4-[4-Oxo-6-(3-phenyl-prop-1-ynyl)-4H-quinazolin-3-ylmethyl]-benzenesulfonamide;

4-[⁴-Oxo-6-(3-phenyl-prop-1-ynyl)-4H-quinazolin-3-ylmethyl]-benzonitrile;

3-(3-Chloro-benzyl)-6-(4-phenyl-but-1-ynyl)-3H-quinazolin-4-one;

3-(3-Chloro-benzyl)-6-(3-phenyl-prop-1-ynyl)-3H-quinazolin-4-one;

4-[4-Oxo-6-(3-pyrazol-1-yl-prop-1-ynyl)-4H-quinazolin-3-ylmethyl]-benzoic acid;

6-(3-Phenyl-prop-1-ynyl)-3-[4-(1H-tetrazol-5-yl)-benzyl]-3H-quinazolin-4-one;

3-(3,4-Difluoro-benzyl)-6-[3-(pyridin-4-yloxy)-prop-1-ynyl]-3H-quinazolin-4-one;

3-(3,4-Difluoro-benzyl)-6-[3-(4-methoxy-phenyl)-prop-1-ynyl]-3H-quinazolin-4-one;

N-{4-[4-Oxo-6-(3-phenyl-prop-1-ynyl)-4H-quinazolin-3-ylmethyl]-phenyl}-acetamide;

3-(3,4-Difluoro-benzyl)-6-(3-phenyl-prop-1-ynyl)-3H-quinazolin-4-one;

3-(4-Acetyl-benzyl)-6-[3-(4-methoxy-phenyl)-prop-1-ynyl]-3H-quinazolin-4-one;

6-(3-Phenyl-prop-1-ynyl)-3-pyridin-4-ylmethyl-3H-quinazolin-4-one; and

6-[3-(4-Methoxy-phenyl)-prop-1-ynyl]-3-pyridin-4-ylmethyl-3H-quinazolin-4-one; or

(iii) Methyl 4-[4-Oxo-6-(3-phenyl-prop-1-ynyl)4H-quinazolin-3-ylmethyl] benzoate;

4-[4-Oxo-6-(3-phenyl-prop-1-ynyl)-4H-quinazolin-3-ylmethyl]-benzoic acid;

4-[6-(3-Methoxy-benzylcarbamoyl)-4-oxo-4H-quinazolin-3-ylmethyl] benzoic acid;

4-{6-[3-(4-Methoxy-phenyl)-prop-1-ynyl]-4-oxo-4H-quinazoline-3-ylmethyl}-benzoic acid;

3-(3-Fluoro-benzyl)-4-oxo-3,4-dihydro-pyrido[3 ,4-d]pyrimidine-6 carboxylic acid 3-methoxy-benzylamide;

3-(3-Fluoro-benzyl)-4-oxo-3,4-dihydro-pyrido[3 ,4-d]pyrimidine-6 carboxylic acid 4-methoxy-benzylamide;

4-[6-(3-Imidazol-1-yl-prop-1-ynyl)-4-oxo-4H-quinazolin-3-ylmethyl]-benzoic acid;

4-[4-Oxo-6-(3-phenyl-prop-1-ynyl)-4H-quinazolin-3-ylmethyl]-benzenesulfonamide;

4-[4-Oxo-6-(3-phenyl-prop-1-ynyl)-4H-quinazolin-3-ylmethyl]-benzonitrile;

6-(3-Phenyl-prop-1-ynyl)-3-[4-(1H-tetrazol-5-yl)-benzyl]-3H-quinazolin-4-one;

3-(3,4-Difluoro-benzyl)-6-[3-(pyridin-4-yloxy)-prop-1-ynyl]-3H-quinazolin-4-one;

3-(3,4-Difluoro-benzyl)-6-[3-(4-methoxy-phenyl)-prop-1-ynyl]-3H-quinazolin-4-one;

N-{4-[4-Oxo-6-(3-phenyl-prop-1-ynyl)-4H-quinazolin-3-ylmethyl]-phenyl}-acetamide;

3-(4-Acetyl-benzyl)-6-[3-(4-methoxy-phenyl)-prop-1-ynyl]-3H-quinazolin-4-one;

6-(3-Phenyl-prop-1-ynyl)-3-pyridin-4-ylmethyl-3H-quinazolin-4-one; and

6-[3-(4-Methoxy-phenyl)-prop-1-ynyl]-3-pyridin-4-ylmethyl-3H-quinazolin-4-one;

Still another aspect of this invention is a combination, pharmaceutical composition, or method of this invention, wherein the allosteric inhibitor of MMP-13, or a pharmaceutically acceptable salt thereof, is selected from:

3-benzyl-2,4-dioxo-3,4-dihydro-2H-benzo[e][1,3]thiazine-6-carboxylic acid 4-methoxy benzylamide;

3-(4-methoxybenzyl)2,4-dioxo-3,4-dihydro-2H-benzo[e][1,3]oxazine-6-carboxylic acid 4-methoxybenzylamide; and

and 4-[2,4-dioxo-6-(3-phenyl-prop-1-ynyl)-4H-1,3-benzothiazin-3-ylmethyl]-benzoic acid;

or a pharmaceutically acceptable salt thereof.

Still another aspect of this invention is a combination, pharmaceutical composition, or method of this invention, wherein the allosteric inhibitor of MMP-13 is a compound of Formula IB

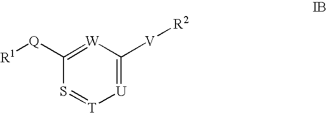

or a pharmaceutically acceptable salt thereof,

wherein:

R¹ and R² independently are selected from: H, C₁-C₆ alkyl, Substituted C₁-C₆ alkyl, C₂-C₆ alkenyl, Substituted C₂-C₆ alkenyl, C₂-C₆ alkynyl, Substituted C₂-C₆ alkynyl, C₃-C₆ cycloalkyl, Substituted C₃-C₆ cycloalkyl, C₃-C₆ cycloalkyl-(C₁-C₆ alkylenyl), Substituted C₃-C₆ cycloalkyl-(C₁-C₆ alkylenyl), 3-to 6-membered heterocycloalkyl, Substituted 3-to 6-membered heterocycloalkyl, 3-to 6-membered heterocycloalkyl-(C₁-C₆ alkylenyl), Substituted 3-to 6-membered heterocycloalkyl-(C₁-C₆ alkylenyl), Phenyl-(C₁-C₆ alkylenyl), Substituted phenyl-(C₁-C₆ alkylenyl), Naphthyl-(C₁-C₆ alkylenyl), Substituted naphthyl-(C₁-C₆ alkylenyl), 5-, 6-, 9-, and 10-membered heteroaryl-(C₁-C₆ alkylenyl), Substituted 5-, 6-, 9-, and 10-membered heteroaryl-(C₁-C₆ alkylenyl), Phenyl, Substituted phenyl, Naphthyl, Substituted naphthyl, 5-, 6-, 9-, and 10-membered heteroaryl, Substituted 5-, 6-, 9-, and 10-membered heteroaryl, R³O-(C₁-C₆ alkylenyl), Substituted R³O-(C₁-C₆ alkylenyl), Phenyl, Substituted phenyl, Naphthyl, Substituted naphthyl, 5- or 6-membered heteroaryl, Substituted 5- or 6-membered heteroaryl, 8-to 10-membered heterobiaryl, Substituted 8-to 10-membered heterobiaryl, Phenyl-O-(C₁-C₈ alkylenyl), Substituted phenyl-O-(C₁-C₈ alkylenyl), Phenyl-S-(C₁-C₈ alkylenyl), Substituted phenyl-S-(C₁-C₈ alkylenyl), Phenyl-S(O)-(C₁-C₈ alkylenyl), Substituted phenyl-S(O)-(C₁-C₈ alkylenyl), Phenyl-S(0)₂-(C₁-C₈ alkylenyl), and Substituted phenyl-S(O)₂-(C₁-C8 alkylenyl);

wherein R₁ and R² are not both selected from: H, C₁-C₆ alkyl, C₂-C₆ alkenyl, C₂-C₆ alkynyl, and C₃-C₆ cycloalkyl;

Each R³ independently is selected from: H, C₁-C₆ alkyl, Substituted C₁-C₆ alkyl, C₃-C₆ cycloalkyl, Substituted C₃-C₆ cycloalkyl, Phenyl-(C₁-C₆ alkylenyl), Substituted phenyl-(C₁-C₆ alkylenyl), Naphthyl-(C₁-C₆ alkylenyl), Substituted naphthyl-(C₁-C₆ alkylenyl), 5-, 6-, 9-, and 10-membered heteroaryl-(C₁-C₆ alkylenyl), Substituted 5-, 6-, 9-, and 10-membered heteroaryl-(C₁-C₆ alkylenyl), Phenyl, Substituted phenyl, Naphthyl, Substituted naphthyl, 5-, 6-, 9-, and 10-membered heteroaryl, Substituted 5-, 6-, 9-, and 10-membered heteroaryl;

S, T, U, and W each are C-R⁴; or

One of S, T, U, and W is N and the other three of S, T, U, and W are C-R⁴; or

Two of S, T, U, and W are N and the other two of S, T, U, and W are C-R⁴; or

T is C-R⁴ and S, U, and W are each N; or

U is C-R⁴ and S, T, and W are each N; or

S is C-R⁴ and T, U, and W are each N;

Each R⁴independently is selected from: H, F, CH₃, CF₃, C(O)H, CN, HO, CH₃O, C(F)H₂O, C(H)F₂O, and CF₃O;

V is a 5-membered heteroarylenyl; and

Q is selected from: OCH₂, N(R⁶)CH₂, OC(O), CH(R⁶)C(O), OC(NR⁶), CH(R⁶)C(NR⁶), N(R⁶)C(O), N(R⁶)C(S), N(R⁶)C(NR⁶), N(R⁶)CH₂, SC(O), CH(R⁶)C(S), SC(NR⁶), trans-(H)C=C(H), cis-(H)C=C(H), C≡C, CH₂C≡C, C≡CCH₂, CF₂C≡C, C≡CCF₂, 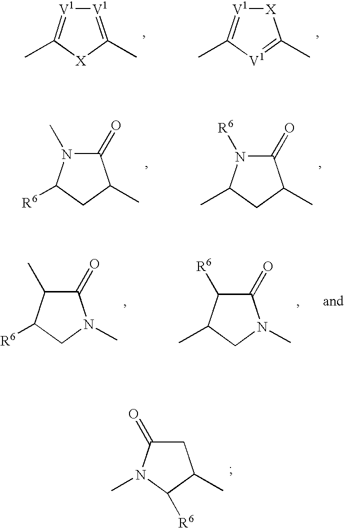
or

V is C(O)O, C(S)O, C(O)N(R⁵), or C(S)N(R⁵); and

Q is selected from: OCH₂, N(R⁶)CH₂, CH(R⁶)C(O), OC(NR⁶), CH(R⁶)C(NR⁶), N(R⁶)C(NR⁶), N(R⁶)CH₂, CH(R⁶)C(S), SC(NR⁶), trans-(H)C=C(H), cis-(H)C=C(H), C≡CCH₂, C≡CCF₂,

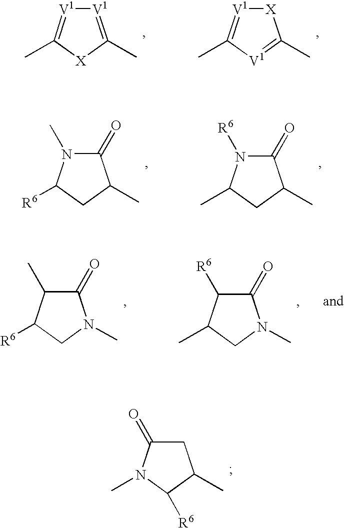

R⁵ is H or C₁-C₆ alkyl;

R⁶ is H, C₁-C₆ alkyl, C₃-C₆ cycloalkyl, 3-to 6-membered heterocycloalkyl, phenyl, benzyl, or 5- or 6-membered heteroaryl;

X is O, S, N(H), or N(C₁-C₆ alkyl);

Each V¹ is independently C(H) or N;

Each “substituted” group contains from 1 to 4 substituents, each independently on a carbon or nitrogen atom, independently selected from: C₁-C₆ alkyl, C₂-C₆ alkenyl, C₂-C₆ alkynyl, C₃-C₆ cycloalkyl, C₃-C₆ cycloalkylmethyl, Phenyl, Phenylmethyl, 3-to 6-membered heterocycloalkyl, 3-to 6-membered heterocycloalkylmethyl, cyano, CF₃, (C₁-C₆ alkyl)-OC(O), HOCH₂, (C₁-C₆ alkyl)-OCH₂, H₂NCH₂, (C₁-C₆ alkyl)—NH(H)CH₂, (C₁-C₆ alkyl)₂-NCH₂, N(H)₂C(O), (C₁-C₆ alkyl)—NH(H)C(O), (C₁-C₆ alkyl)₂-NC(O), N(H)₂C(O)N(H), (C₁-C₆ alkyl)—NH(H)C(O)N(H), N(H)₂C(O)N(C₁-C₆ alkyl), (C₁-C₆ alkyl)—NH(H)C(O)N(C₁-C₆ alkyl), (C₁-C₆ alkyl)₂-NC(O)N(H), (C₁-C₆ alkyl)₂-NC(O)N(C₁-C₆ alkyl), N(H)₂C(O)O, (C₁-C₆ alkyl)-N(H)C(O)O, (C₁-C₆ alkyl)₂-NC(O)O, HO, (C₁-C₆ alkyl)-O, CF₃O, CF₂(H)O, CF(H)₂O, H₂N, (C₁-C₆ alkyl)—NH(H), (C₁-C₆ alkyl)₂-N, O₂N, (C₁-C₆ alkyl)-S, (C₁-C₆ alkyl)-S(O), (C₁-C₆ alkyl)-S(O)₂, (C₁-C₆ alkyl)₂-NS(O)₂, (C₁-C₆ alkyl)-S(O)₂—NH(H)—C(O)-(C₁-C₈ alkylenyl)ₘ, and (C₁-C₆ alkyl)—C(O)—NH(H)-S(O)₂-(C₁-C8 alkylenyl)ₘ;

wherein each substituent on a carbon atom may further be independently selected from: Halo, HO₂C, and OCH₂O, wherein each O is bonded to adjacent carbon atoms to form a 5-membered ring;

wherein 2 substituents may be taken together with a carbon atom to which they are both bonded to form the group C═O;

wherein two adjacent, substantially sp² carbon atoms may be taken together with a diradical substituent to form a cyclic diradical selected from:

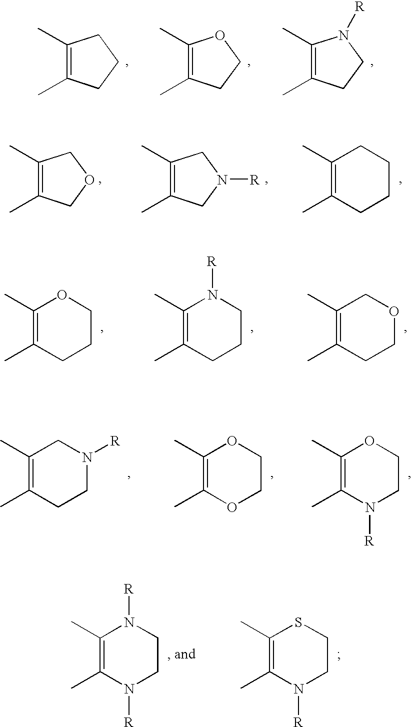

R is H or C₁-C₆ alkyl;

wherein each m independently is an integer of 0 or 1;

wherein each 5-membered heteroarylenyl independently is a 5-membered ring containing carbon atoms and from 1 to 4 heteroatoms selected from 1 O, 1 S, 1 NH, 1 N(C₁-C₆ alkyl), and 4 N, wherein the O and S atoms are not both present, and wherein the heteroarylenyl may optionally be unsubstituted or substituted with 1 substituent selected from fluoro, methyl, hydroxy, trifluoromethyl, cyano, and acetyl;

wherein each heterocycloalkyl is a ring that contains carbon atoms and 1 or 2 heteroatoms independently selected from 2 O, 1 S, 1 S(O), 1 S(O)₂, 1 N, 2 N(H), and 2 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond;

wherein each 5-membered heteroaryl contains carbon atoms and from 1 to 4 heteroatoms independently selected from O, 1 S, 1 N(H), 1 N(C₁-C₆ alkyl), and 4 N, and each 6-membered heteroaryl contains carbon atoms and 1 or 2 heteroatoms independently selected from N, N(H), and N(C₁-C₆ alkyl), and 5- and 6-membered heteroaryl are monocyclic rings; and 9- and 10-membered heteroaryl are 6,5-fused and 6,6-fused bicyclic rings, respectively, wherein at least 1 of the 2 fused rings of a bicyclic ring is aromatic, and wherein when the O and S atoms both are present, the O and S atoms are not bonded to each other;

wherein with any (C₁-C₆ alkyl)₂-N group, the C₁-C₆ alkyl groups may be optionally taken together with the nitrogen atom to which they are attached to form a 5- or 6-membered heterocycloalkyl; and

wherein each group and each substituent recited above is independently selected.

Still another aspect of this invention is a combination, pharmaceutical composition, or method of this invention, wherein the allosteric inhibitor of MMP-13 is a compound of Formula IB, or a pharmaceutically acceptable salt thereof, wherein

(i) V is selected from the groups:

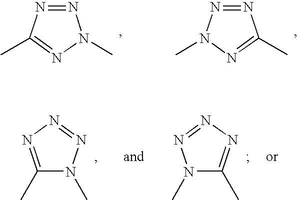

(ii) Q is C≡C, CH₂C≡C, or CF₂C≡C; or

(iii) Q is N(R⁶)C(O); or

(iv) each of R¹ and R² are independently selected from:
Phenyl-(C₁-C₆ alkylenyl); and
Substituted phenyl-(C₁-C₆ alkylenyl);

wherein each group and each substituent is independently selected; or

(v) each of R¹ and R² is independently selected from:
5-, 6-, 9-, and 10-membered heteroaryl-(C₁-C₆ alkylenyl); and
Substituted 5-, 6-, 9-, and 10-membered heteroaryl-(C₁-C₆ alkylenyl);

wherein each heteroaryl contains carbon atoms and from 1 to 4 heteroatoms independently selected from 1 O, 1 S, 1 N(H), 1 N(C₁-C₆ alkyl), and 4 N, and 5- and 6-membered heteroaryl are monocyclic rings and 9- and 10-membered heteroaryl are 6,5-fused and 6,6-fused bicyclic rings, respectively, wherein at least 1 of the 2 fused rings of a bicyclic ring is aromatic, and wherein when the O and S atoms both are present, the O and S atoms are not bonded to each other; and

wherein each group and each substituent is independently selected; or

(vi) wherein S, T, U, and W are each CH.

Still another aspect of this invention is a combination, pharmaceutical composition, or method of this invention, the allosteric inhibitor of MMP-13 is a compound of Formula IB as drawn above, or a pharmaceutically acceptable salt thereof, wherein:

each of R¹ and R² is independently selected from:
Phenyl-(C₁-C₆ alkylenyl); and
Substituted phenyl-(C₁-C₆ alkylenyl);
5-, 6-, 9-, and 10-membered heteroaryl-(C₁-C₆ alkylenyl); and
Substituted 5-, 6-, 9-, and 10-membered heteroaryl-(C₁-C₆ alkylenyl);

S, T, U, and W each are C-R⁴; or

One of S, T, U, and W is N and the other three of S, T, U, and W are C-R⁴;

Each R⁴ independently is selected from: H, CH₃, and OCH₃;

V is a 5-membered heteroarylenyl which is:

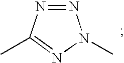

Q is selected from: N(H)C(O);

Each “substituted” group contains from 1 to 4 substituents, each independently on a carbon or nitrogen atom, independently selected from: C₁-C₆ alkyl, C₂-C₆ alkenyl, C₂-C₆ alkynyl, C₃-C₆ cycloalkyl, C₃-C₆ cycloalkylmethyl, Phenyl, Phenylmethyl, 3-to 6-membered heterocycloalkyl, 3-to 6-membered heterocycloalkylmethyl, cyano, CF₃, (C₁-C₆ alkyl)-OC(O), HOCH₂, (C₁-C₆ alkyl)-OCH₂, H₂NCH₂, (C₁-C₆ alkyl)—NH(H)CH₂, (C₁-C₆ alkyl)₂-NCH₂, N(H)₂C(O), (C₁-C₆ alkyl)—NH(H)C(O), (C₁-C₆ alkyl)₂-NC(O), N(H)₂C(O)N(H), (C₁-C₆ alkyl)—NH(H)C(O)N(H), N(H)₂C(O)N(C₁-C₆ alkyl), (C₁-C₆ alkyl)—NH(H)C(O)N(C₁-C₆ alkyl), (C₁-C₆ alkyl)₂-NC(O)N(H), (C₁-C₆ alkyl)₂-NC(O)N(C₁-C₆ alkyl), N(H)₂C(O)O, (C₁-C₆ alkyl)—NH(H)C(O)O, (C₁-C₆ alkyl)₂-NC(O)O, HO, (C₁-C₆ alkyl)-O, CF₃O, CF₂(H)O, CF(H)₂O, H₂N, (C₁-C₆ alkyl)—NH(H), (C₁-C₆ alkyl)₂-N, O₂N, (C₁-C₆ alkyl)-S, (C₁-C₆ alkyl)-S(O), (C₁-C₆ alkyl)-S(O)₂, (C₁-C₆ alkyl)₂-NS(O)₂, (C₁-C₆ alkyl)-S(O)₂—NH(H)—C(O)-(C₁-C₈ alkylenyl)ₘ, and

(C₁-C₆ alkyl)—C(O)—NH(H)-S(O)₂-(C₁-C₈ alkylenyl)ₘ;

wherein each substituent on a carbon atom may further be independently selected from: Halo, HO₂C, and OCH₂O, wherein each O is bonded to adjacent carbon atoms to form a 5-membered ring;

wherein 2 substituents may be taken together with a carbon atom to which they are both bonded to form the group C═O;

wherein each m independently is an integer of 0 or 1;

wherein each 5-membered heteroarylenyl independently is a 5-membered ring containing carbon atoms and from 1 to 4 heteroatoms selected from 1 O, 1 S, 1 NH, 1 N(C₁-C₆ alkyl), and 4 N, wherein the O and S atoms are not both present, and wherein the heteroarylenyl may optionally be unsubstituted or substituted with 1 substituent selected from fluoro, methyl, hydroxy, trifluoromethyl, cyano, and acetyl;

wherein each heterocycloalkyl is a ring that contains carbon atoms and 1 or 2 heteroatoms independently selected from 2 O, 1 S, 1 S(O), 1 S(O)₂, 1 N, 2 N(H), and 2 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond;

wherein each 5-membered heteroaryl contains carbon atoms and from 1 to 4 heteroatoms independently selected from O, 1 S, 1 N(H), 1 N(C₁-C₆ alkyl), and 4 N, and each 6-membered heteroaryl contains carbon atoms and 1 or 2 heteroatoms independently selected from N, N(H), and N(C₁-C₆ alkyl), and 5- and 6-membered heteroaryl are monocyclic rings; and 9- and 10-membered heteroaryl are 6,5-fused and 6,6-fused bicyclic rings, respectively, wherein at least 1 of the 2 fused rings of a bicyclic ring is aromatic, and wherein when the O and S atoms both are present, the O and S atoms are not bonded to each other;

wherein with any (C₁-C₆ alkyl)₂-N group, the C₁-C₆ alkyl groups may be optionally taken together with the nitrogen atom to which they are attached to form a 5- or 6-membered heterocycloalkyl; and

wherein each group and each substituent recited above is independently selected. Preferred is S is N and T, U, and W are each C-R⁴, wherein R⁴ is as defined above (i.e., each R⁴ independently is H, CH₃, or OCH₃). Also preferred is each C₁-C₆ alkylenyl is independently CH₂ or C(═O). Also preferred is each C₁-C₆ alkyl independently is CH₃ or CH₂CH₃. Also preferred is each C₁-C₈ alkylenyl independently is CH₂ or CH₂CH₂. Also preferred is each m is 0. Also preferred is one m is 1 and each other m is 0. Also preferred is each R¹ and R² is independently a substituted phenyl-(C₁-C₆ alkylenyl) or a substituted 5-, 6-, 9-, or 10-membered heteroaryl-(C₁-C₆ alkylenyl).

Still another aspect of this invention is a combination, pharmaceutical composition, or method of this invention, wherein the allosteric inhibitor of MMP-13 is a compound of Formula IB, or a pharmaceutically acceptable salt thereof, selected from:

4-({3-[2-(4-Methoxy-benzyl)-2H-tetrazol-5-yl]-benzoylamino}-methyl)-benzoic acid methyl ester;

4-({3-[2-(4-Methoxy-benzyl)-2H-tetrazol-5-yl]-benzoylamino }-methyl)-benzoic acid;

4-({3-[2-(3-Methoxy-benzyl)-2H-tetrazol-5-yl]-benzoylamino }-methyl)-benzoic acid methyl ester;

4-({3-[2-(3-Methoxy-benzyl)-2H-tetrazol-5-yl]-benzoylamino}-methyl)-benzoic acid;

3-[2-(4-Methoxy-benzyl)-2H-tetrazol-5-yl]-N-(4-morpholin-4-ylmethyl-benzyl)-benzamide;

3-[2-(4-Methoxy-benzyl)-2H-tetrazol-5-yl]-N-(3-trifluoromethyl-benzyl)-benzamide;

N-Benzyl-3-[2-(4-methoxy-benzyl)-2H-tetrazol-5-yl]-benzamide;

3-[2-(4-Methoxy-benzyl)-2H-tetrazol-5-yl]-N-(2-trifluoromethyl-benzyl)-benzamide; and

N-(4-Methoxy-benzyl)-3-[2-(4-methoxy-benzyl)-2H-tetrazol-5-yl]-benzamide;

or a pharmaceutically acceptable salt thereof.

Still another aspect of this invention is a combination, pharmaceutical composition, or method of this invention, wherein the allosteric inhibitor of MMP-13, or a pharmaceutically acceptable salt thereof, is selected from:

4-({3-[2-(4-Fluoro-benzyl)-2H-tetrazol-5-yl]-benzoylamino}-methyl)-benzoic acid methyl ester;

4-({3-[2-(4-Fluoro-benzyl)-2H-tetrazol-5-yl]-benzoylamino}-methyl)-benzoic acid;

4-({3-[2-(3-Fluoro-benzyl)-2H-tetrazol-5-yl]-benzoylamino}-methyl)-benzoic acid methyl ester;

4-({3-[2-(3-Fluoro-benzyl)-2H-tetrazol-5-yl]-benzoylamino}-methyl)-benzoic acid;

N-(3-Chloro-4-fluoro-benzyl)-3-[2-(4-methoxy-benzyl)-2H-tetrazol-5-yl]-benzamide;

N-(2,3-Difluoro-benzyl)-3-[2-(4-methoxy-benzyl)-2H-tetrazol-5-yl]-benzamide; and

N-(4-Fluoro-benzyl)-2-methoxy-5-[2-(4-methoxy-benzyl)-2H-tetrazol-5-yl]-benzamide; or

a pharmaceutically acceptable salt thereof.

Still another aspect of this invention is a combination, pharmaceutical composition, or method of this invention, wherein the allosteric inhibitor of MMP-13, or a pharmaceutically acceptable salt thereof, is selected from:

N-Benzyl-3-[2-(4-cyano-benzyl)-2H-tetrazol-5-yl]-benzamide;

4-{[3-(2-Thiazol-2-ylmethyl-2H-tetrazol-5-yl)-benzoylamino]-methyl}-benzoic acid methyl ester;

4-{[3-(2-But-2-enyl-2H-tetrazol-5-yl)-benzoylamino]-methyl}benzoic acid methyl ester;

N-Benzyl-3-(2-but-2-enyl-2H-tetrazol-5-yl)-benzamide;

3-(2-But-2-enyl-2H-tetrazol-5-yl)-N-(3-methoxy-benzyl)-benzamide;

3-[2-(4-Methoxy-benzyl)-2H-tetrazol-5-yl]-N-thiazol-2-ylmethyl-benzamide;

N-2,1,3-Benzothiadiazol-5-ylmethyl-3-[2-(4-methoxy-benzyl)-2H-tetrazol-5-yl]-benzamide;

3-[2-(4-Methoxy-benzyl)-2H-tetrazol-5-yl]-N-(2-methoxy-pyridin-4-ylmethyl)-benzamide;

3-[2-(4-Methoxy-benzyl)-2H-tetrazol-5-yl]-N-pyridin-4-ylmethyl-benzamide;

N-1,3-Benzodioxol-5-ylmethyl-3-[2-(4-methoxy-benzyl)-2H-tetrazol-5-yl]-benzamide; and

N-Furan-2-ylmethyl-3-[2-(4-methoxy-benzyl)-2H-tetrazol-5-yl]-benzamide; or

a pharmaceutically acceptable salt thereof.

Still another aspect of this invention is a combination, pharmaceutical composition, or method of this invention, wherein the allosteric inhibitor of MMP-13, or a pharmaceutically acceptable salt thereof, is selected from:

4-(5-{3-[(Pyridin-4-ylmethyl)-carbamoyl]-phenyl }-tetrazol-2-ylmethyl)-benzoic acid;

4-(5-{3-[(Pyridin-3-ylmethyl)-carbamoyl]-phenyl }-tetrazol-2-ylmethyl)-benzoic acid;

4-(5- {3-[(2-Methoxy-pyridin-4-ylmethyl)-carbamoyl]-phenyl }-tetrazol-2-ylmethyl)-benzoic acid;

3-[2-(4-Methoxy-benzyl)-2H-tetrazol-5-yl]-N-(2-pyridin-4-yl-ethyl)-benzamide;

N-Isopropyl-3-[2-(4-methoxy-benzyl)-2H-tetrazol-5-yl]-benzamide;

3-[2-(4-Methoxy-benzyl)-2H-tetrazol-5-yl]-N-( 1-phenyl-ethyl)-benzamide;

4-(5-{3-[(Methyl-pyridin-3-ylmethyl)-carbamoyl]-phenyl}-tetrazol-2-ylmethyl)-benzoic acid;

4-({2-Methoxy-5-[2-(4-methoxy-benzyl)-2H-tetrazol-5-yl]-benzoylamino}ylmethyl)-benzoic acid;

4-({2-Methoxy-5-[2-(4-methoxy-benzyl)-2H-tetrazol-5-yl]-benzoylamino}-methyl)-benzoic acid; and

2-Methoxy-5-[2-(4-methoxy-benzyl)-2H-tetrazol-5-yl]-N-(4-trifluoromethyl-benzyl)-benzamide;

or a pharmaceutically acceptable salt thereof.

Still another aspect of this invention is a combination, pharmaceutical composition, or method of this invention, wherein the allosteric inhibitor of MMP-13, or a pharmaceutically acceptable salt thereof, is selected from:

Benzyl{3-[2-(4-methoxy-benzyl)-2H-tetrazol-5-yl]-benzyl}-amine hydrochloride;

(4-Methanesulfonyl-benzyl)-{3-[2-(4-methoxy-benzyl)-2H-tetrazol-5-yl]-benzyl}-amine;

4-({3-[2-(4-Methoxy-benzyl)-2H-tetrazol-5yl]-benzylamino}-methyl)-benzoic acid;

4-{3-[2-(4-Methoxy-benzyl)-2H-tetrazol-5-yl]-benzyloxymethyl}-benzoic acid;

4-{3-[2-(4-Methoxy-benzyl)-2H-tetrazol-5-yl]-benzyloxy}-benzoic acid;

4-{5-[3-(3-Phenyl-prop-1-ynyl)-phenyl]-tetrazol-2-ylmethyl}-benzoic acid;

4-(5-{3-[3-(4-Fluoro-phenyl)-prop-1-ynyl]-phenyl}-tetrazol-2-ylmethyl)-benzoic acid;

4-{5-[3-(3-Methyl-3-phenyl-but-1-ynyl)-phenyl]-tetrazol-2-ylmethyl}-benzoic acid; and

4-{5-[3-(3-Imidazol-1-yl-prop-1-ynyl)-phenyl]-terazol-2-ylmethyl}-benzoic acid;

or a pharmaceutically acceptable salt thereof.

Still another aspect of this invention is a combination, pharmaceutical composition, or method of this invention, wherein the allosteric inhibitor of MMP-13, or a pharmaceutically acceptable salt thereof, is selected from:

4-[1-Oxo-7-(3-phenyl-prop-1-ynyl)-1H-isoquinolin-2-ylmethyl]benzoic acid tert-butyl ester;

4-[1-Oxo-7-(3-phenyl-prop-1-ynyl)-1H-isoquinolin-2-ylmethyl]benzoic acid methyl ester;

3-[1-Oxo-7-(3-phenyl-prop-1-ynyl)-1H-isoquinolin-2-ylmethyl]benzoic acid methyl ester;

4-[7-(3-Imidazol-1-ylprop-1-ynyl)-1-oxo-1H-isoquinolin-2-ylmethyl]benzoic acid tert-butyl ester;

4-[1-Oxo-7-(3-[1,2,3]triazol-1-ylprop-1-ynyl)-1H-isoquinolin-2-ylmethyl]benzoic acid tert-butyl ester;

4-[1 -Oxo-7-(3-[1,2,4]triazol-1-ylprop-1 -ynyl)-1H-isoquinolin-2-ylmethyl]benzoic acid tert-butyl ester;

4-[4-Methyl-1-oxo-7-(3-phenylprop-1-ynyl)-1H-isoquinolin-2-ylmethyl]benzoic acid tert-butyl ester;

4-[4-Methyl-1-oxo-7-(3-phenylprop-1-ynyl)-1H-isoquinolin-2-ylmethyl]benzoic acid tert-butyl ester; or

a pharmaceutically acceptable salt thereof.

Still another aspect of this invention is a combination, pharmaceutical composition, or method of this invention, wherein the allosteric inhibitor of MMP-13, or a pharmaceutically acceptable salt thereof, is selected from:

4-[1-Oxo-7-(3-phenyl-prop-1-ynyl)-1H-isoquinolin-2-ylmethyl]benzoic acid;

7-(3-Phenyl-prop-1-ynyl)-2-(4-trifluoromethylbenzyl)-2H-isoquinolin-1-one;

2-(3-Fluorobenzyl)-7-(3-phenyl-prop-1-ynyl)-2H-isoquinolin-1-one;

3-[1-Oxo-7-(3-phenyl-prop-1-ynyl)-1H-isoquinolin-2-ylmethyl]benzonitrile;

4-[1-Oxo-7-(3-phenyl-prop-1-ynyl)-1H-isoquinolin-2-ylmethyl]benzenesulfonamide;

2-(4-Fluorobenzyl)-7-3-phenylprop-1-ynyl-2H-isoquinolin-1-one;

7-(3-Phenylprop-1-ynyl)-2-(3-trifluoromethylbenzyl)-2H-isoquinolin-1-one;

2-(3-Chlorobenzyl)-7-(3-phenylprop-1-ynyl)-2H-isoquinolin-1-one;

2-(3,4-Difluorobenzyl)-7-(3-phenylprop-1-ynyl)-2H-isoquinolin-1-one;

2-(3,5-Difluoro-4-hydroxybenzyl)-7-[3-(4H-[1 ,2,3]triazol-4-yl)prop-1-ynyl]-2H-isoquinolin-1-one;

4-[7-(3-Imidazol-1-ylprop-1-ynyl)-1-oxo-1H-isoquinolin-2-ylmethyl]benzoic acid; acid;

4-[1-Oxo-7-(3-[1,2,4]triazol-1-ylprop-1 -ynyl)- lH-isoquinolin-2-ylmethyl]benzoic acid;

4-[4-Methyl-1-oxo-7-(3-phenylprop-1-ynyl)- 1H-isoquinolin-2-ylmethyl]benzoic acid;

4-[4-Methyl-1-oxo-7-(3-phenylprop-1-ynyl)-1H-isoquinolin-2-ylmethyl]benzoic acid; or

a pharmaceutically acceptable salt thereof.

Still another aspect of this invention is a combination, pharmaceutical composition, or method of this invention, wherein the allosteric inhibitor of MMP-13, or a pharmaceutically acceptable salt thereof, is selected from:

2,2-Dioxo-3-[1-phenyl-meth-(Z)-ylidene]-6-(3-phenyl-prop-1-ynyl)-2,3-dihydro-1λ⁶-benzo[c][1,2]thiazin-4-one; or

a pharmaceutically acceptable salt thereof.

Still another aspect of this invention is a combination, pharmaceutical composition, or method of this invention, wherein the allosteric inhibitor of MMP-13, or a pharmaceutically acceptable salt thereof, is selected from:

3-(4-Methanesulfonyl-benzyl)-1-methyl-6-(3-phenyl-prop-1-ynyl)-3,4-dihydro-1H-quinazolin-2-one;

or a pharmaceutically acceptable salt thereof.

Still another aspect of this invention is a combination, pharmaceutical composition, or method of this invention, wherein the allosteric inhibitor of MMP-13, or a pharmaceutically acceptable salt thereof, is selected from:

4-[4-(Biphenyl-3-ylmethylsulfanyl)-5-methyl-2,6-dioxo-3,6-dihydro-2H-pyrimidin-1-ylmethyl]-benzoic acid;

3-Benzyl-6-(biphenyl-3-ylmethanesulfinyl)-5-methyl-1H-pyrimidine-2,4-dione;

3-Benzyl-6-(biphenyl-3-ylmethylsulfanyl)-5-methyl-1H-pyrimidine-2,4-dione;

3-Benzyl-6-(biphenyl-4-ylmethylsulfanyl)-5-methyl-1H-pyrimidine-2,4-dione;

3-Benzyl-6-{2-[3-(2,4-dichloro-phenyl)-isoxazol-5-yl]-2-oxo-ethylsulfanyl}-5-methyl-1H-pyrimidine-2,4-dione;

3-Benzyl-6-[5-(4-chloro-phenyl)-isoxazol-3-ylmethylsulfanyl]-5-methyl-1H-pyrimidine-2,4-dione;

3-Benzyl-6-[3-(4-methoxy-phenyl)-isoxazol-5-ylmethylsulfanyl]-5-methyl-1H-pyrimidine-2,4-dione;

3-Benzyl-6-[3-(2,6-dichloro-phenyl)-isoxazol-5-ylmethylsulfanyl]-5-methyl-1H-pyrimidine-2,4-dione;

3-Benzyl-6-[5-(2-chloro-phenyl)-isoxazol-3-ylmethylsulfanyl]-5-methyl-1H-pyrimidine-2,4-dione;

3-Benzyl-6-[2-(4-chloro-phenyl)-thiazol4-ylmethylsulfanyl]-5-methyl-1H-pyrimidine-2,4-dione;

3-Benzyl-6-[5-(4-methoxy-phenyl)-[1 ,2,4]oxadiazol-3-ylmethylsulfanyl]-5-methyl- 1H-pyrimidine-2,4-dione;

3-Benzyl-6-[3-(4-chloro-phenyl)-[1 ,2,4]oxadiazol-5-ylmethylsulfanyl]-5-methyl-1H-pyrimidine-2,4-dione;

3-Benzyl-6-[3-(4-chloro-phenyl)-isoxazol-5-ylmethylsulfanyl]-5-methyl-1H-pyrimidine-2,4-dione;

6-(4-Amino-5-phenyl-4H-[1 ,2,4]triazol-3-ylsulfanyl)-3 -benzyl-5-methyl-1H-pyrimidine-2,4-dione;

3-Benzyl-5-methyl-6-[5-(2-methylsulfanyl-pyridin-3-yl)-[1 ,2,4]oxadiazol-3-ylmethylsulfanyl]-1H-pyrimidine-2,4-dione;

3-Benzyl-5-methyl-6-(3-phenyl-isoxazol-5-ylmethylsulfanyl)-1H-pyrimidine-2,4-dione;

3-Benzyl-5-methyl-6-(5-phenyl-isoxazol-3-ylmethylsulfanyl)-1H-pyrimidine-2,4-dione;

3-Benzyl-5-methyl-6-(5-phenyl-[1 ,2,4]oxadiazol-3-ylmethylsulfanyl)-1H-pyrimidine-2,4-dione;

3-Benzyl-5-methyl-6-(2-phenyl-thiazol-4-ylmethylsulfanyl)-1H-pyrimidine-2,4-dione;

3-Benzyl-5-methyl-6-[3-(4-nitro-benzyl)-[1 ,2,4]oxadiazol-5-ylmethylsulfanyl]-1H-pyrimidine-2,4-dione;

3-Benzyl-6-[5-(4-chloro-phenylamino)-2H-[1 ,2,4]triazol-3-ylsulfanyl]-5-methyl-1H-pyrimidine-2,4-dione;

6-(Benzothiazol-2-ylsulfanyl)-3-benzyl-5-methyl- 1H-pyrimidine-2,4-dione;

3-Benzyl-6-(6-methoxy-benzothiazol-2-ylamino)-5-methyl-1H-pyrimidine-2,4-dione; or

a pharmaceutically acceptable salt thereof. St

ill another aspect of this invention is a combination, pharmaceutical composition, or method of this invention, wherein the allosteric inhibitor of MMP-13, or a pharmaceutically acceptable salt thereof, is selected from:

3-Benzyl-5-methyl-6-(4-morpholin-4-yl-phenylamino)-1H-pyrimidine-2,4-dione;

6-(4-Benzooxazol-2-yl-phenoxy)-3-benzyl-5-methyl-1H-pyrimidine-2,4-dione;

3-Benzyl-5-methyl-6-(4-oxazolo[4,5-b]pyridin-2-yl-phenoxy)-1H-pyrimidine-2,4-dione;

3-Benzyl-6-[3-(2,6-dichloro-phenyl)-isoxazol-5-ylmethylsulfanyl]-1,5-dimethyl-1H-pyrimidine-2,4-dione;

3-Benzyl-1,5-dimethyl-6-[5-(3-methyl-4-nitro-phenyl)-[1,3,4]oxadiazol-2-ylmethylsulfanyl]-1H-pyrimidine-2,4-dione;

3-Benzyl-1 ,5-dimethyl-6-[5-naphthalen-2-yl-[1 ,3,4]oxadiazol-2-ylmethylsulfanyl]-1H-pyrimidine-2,4-dione;

3-Benzyl-1 ,5-dimethyl-6-(5-phenyl-isoxazol-3-ylmethylsulfanyl)-1H-pyrimidine-2,4-dione;

3-Benzyl-1 ,5-dimethyl-6-[3-(4-nitro-benzyl)-[1 ,2,4]oxadiazol-5-ylmethylsulfanyl]-1H-pyrimidine-2,4-dione; and

3-Benzyl-6-{3-[4-(4-chloro-benzyloxy)-phenyl]-[1 ,2,4]oxadiazol-5-ylmethylsulfanyl}-5-methyl-1H-pyrimidine-2,4-dione;

or a pharmaceutically acceptable salt thereof.

Still another aspect of this invention is a combination, pharmaceutical composition, or method of this invention, wherein the allosteric inhibitor of MMP-13, or a pharmaceutically acceptable salt thereof, is selected from:

4-{6-[2-(3-Methoxy-phenyl)-acetylamino]-4-oxo-4H-quinazolin-3-ylmethyl}-benzoic acid;

4-{7-Fluoro-6-[2-(3-methoxy-phenyl)-acetylamino]-4-oxo-4H-quinazolin-3-ylmethyl}-benzoic acid;

4-{6-[2-(4-Methoxy-phenyl)-acetylamino]-4-oxo4H-quinazolin-3-ylmethyl}-benzoic acid;

4-{6-[2-(4-Fluoro-phenyl)-acetylamino]-4-oxo-4H-quinazolin-3-ylmethyl}-benzoic acid;

4-{6-[2-(3-Fluoro-phenyl)-acetylamino]-4-oxo-4H-quinazolin-3-ylmethyl}-benzoic acid;

4-{7-fluoro-6-[2-(4-methoxyphenyl)acetylamino]-4-oxo-4H-quinazolin-3-ylmethyl}benzoic acid;

4-[7-fluoro-4-oxo-6-(2-p-tolylacetylamino)-4H-quinazolin-3-ylmethyl]benzoic acid;

4-{7-fluoro-6-[2-(4-hydroxyphenyl)acetylamino]-4-oxo-4H-quinazolin-3-ylmethyl}benzoic acid;

4-{7-[2-(3-Methoxy-phenyl)-acetylamino]-1-oxo-1H-isoquinolin-2-ylmethyl}-benzoic acid;

4-{7-[2-(4-Methoxy-phenyl)-acetylamino]-1-oxo-1H-isoquinolin-2-ylmethyl}-benzoic acid;

4-{7-[2-(3-Fluoro-phenyl)-acetylamino]-1 -oxo- 1H-isoquinolin-2-ylmethyl}-benzoic acid; and

4-{7-[2-(4-Fluoro-phenyl)-acetylamino]-1 -oxo- 1H-isoquinolin-2-ylmethyl}-benzoic acid;

or a pharmaceutically acceptable salt thereof.

Still another aspect of this invention is a combination, pharmaceutical composition, or method of this invention, wherein the allosteric inhibitor of MMP-13, or a pharmaceutically acceptable salt thereof, is selected from:

4-{6-[2-(4-Methoxy-phenyl)-acetylamino]-1-methyl-2,4-dioxo-1,4-dihydro-2H-quinazolin-3-ylmethyl}-benzoic acid;

4-{6-[2-(3-Methoxy-phenyl)-acetylamino]-1-methyl-2,4-dioxo-1,4-dihydro-2H-quinazolin-3-ylmethyl}-benzoic acid;

4-{6-[2-(4-Fluoro-phenyl)-acetylamino]-1 -methyl-2,4-dioxo-1 ,4-dihydro-2H-quinazolin-3-ylmethyl}-benzoic acid;

4-{6-[2-(3-Fluoro-phenyl)-acetylamino]-1 -methyl-2,4-dioxo-1 ,4-dihydro-2H-quinazolin-3-ylmethyl}-benzoic acid;

4-{7-Fluoro-6-[2-(3-methoxy-phenyl)-acetoxy]-4-oxo-4H-quinazolin-3-ylmethyl}-benzoic acid;

4-{6-[2-(3-Methoxy-phenyl)-acetoxy]-4-oxo-4H-quinazolin-3-ylmethyl}-benzoic acid;

4-{6-[2-(4-Methoxy-phenyl)-acetoxy]-4-oxo-4H-quinazolin-3-ylmethyl}-benzoic acid;

4-{6-[2-(3-Fluoro-phenyl)-acetoxy]-4-oxo-4H-quinazolin-3-ylmethyl}-benzoic acid;

4-{6-[2-(4-Fluoro-phenyl)-acetoxy]-4-oxo-4H-quinazolin-3-ylmethyl}-benzoic acid;

4-{7-[2-(4-Fluoro-phenyl)-acetoxy]-1-oxo- 1H-isoquinolin-2-ylmethyl}-benzoic acid;

4-{7-[2-(3-Fluoro-phenyl)-acetoxy]-1-oxo-1H-isoquinolin-2-ylmethyl}-benzoic acid;

4-{7-[2-(4-Methoxy-phenyl)-acetoxy]-1 -oxo-1H-isoquinolin-2-ylmethyl}-benzoic acid;

4-{7-[2-(3-Methoxy-phenyl)-acetoxy]-1 -oxo-1H-isoquinolin-2-ylmethyl}-benzoic acid;

4-{6-[2-(4-Methoxy-phenyl)-acetoxy]-1-methyl-2,4-dioxo-1 ,4-dihydro-2H-quinazolin-3-ylmethyl}-benzoic acid;

5 4-{6-[2-(3-Methoxy-phenyl)-acetoxy]-l-methyl-2,4-dioxo-1 ,4-dihydro-2H-quinazolin-3-ylmethyl}-benzoic acid;

4-{6-[2-(4-Fluoro-phenyl)-acetoxy]-1-methyl-2,4-dioxo-1 ,4-dihydro-2H-quinazolin-3-ylmethyl}-benzoic acid; and

4-{6-[2-(3-Fluoro-phenyl)-acetoxy]-1 -methyl-2,4-dioxo-1 ,4-dihydro-2H-10 quinazolin-3-ylmethyl}-benzoic acid;

or a pharmaceutically acceptable salt thereof.

Still another aspect of this invention is a combination, pharmaceutical composition, or method of this invention, wherein the ligand to the alpha-2-delta receptor, or a pharmaceutically acceptable salt thereof, is a ligand to the alpha-2-delta receptor named in any one of the below listed U.S. patents, U.S. patent applications, U.S. patent application publications, European patents, European patent application publications, or PCT International Patent Application Publications, or a pharmaceutically acceptable salt thereof.

Still another aspect of this invention is a combination, pharmaceutical composition, or method of this invention, wherein the ligand to the alpha-2-delta receptor, or a pharmaceutically acceptable salt thereof, is gabapentin.

Still another aspect of this invention is a combination, pharmaceutical composition, or method of this invention, wherein the ligand to the alpha-2-delta receptor, or a pharmaceutically acceptable salt thereof, is pregabalin.

Still another aspect of this invention is a combination, pharmaceutical composition, or method of this invention, wherein the ligand to the alpha-2-delta receptor, or a pharmaceutically acceptable salt thereof, is (3S,4S)-(1-Aminomethyl-3,4-dimethyl-cyclopentyl)-acetic acid.

Still another aspect of this invention is a combination, pharmaceutical composition, or method of this invention, wherein the ligand to the alpha-2-delta receptor, or a pharmaceutically acceptable salt thereof, is 3-(1-aminomethyl-cyclohexylmethyl)-4H-[1,2,4]oxadiazol-5-one hydrochloride.

Still another aspect of this invention is a combination, pharmaceutical composition, or method of this invention, wherein the ligand to the alpha-2-delta receptor, or a pharmaceutically acceptable salt thereof, is selected from:

3-(1 -aminomethyl-cyclohexylmethyl)-4H-[1 ,2,4]oxadiazol-5-one;

C-[1-(1H-tetrazol-5-ylmethyl)-cycloheptyl]-methylamine;

(3S,5R)-3-aminomethyl-5-methyl-octanoic acid;

(S,S)-(1-aminomethyl-3,4-dimethyl-cyclopentyl)-acetic acid;

[(1R,5R,6S)-6-(aminomethyl)bicyclo[3.2.0]hept-6-yl]acetic acid;

(1α,3α,5α)(3-amino-methyl-bicyclo[3.2.0]hept-3-yl)-acetic acid;

(3S,5R)-3-amino-5-methyl-nonanoic acid;

(3S,5R)-3-amino-5-methyl-octanoic acid;

(3S,5R)-3-amino-5-methyl-heptanoic acid; or

a pharmaceutically acceptable salt thereof.

Still another aspect of this invention is a combination, pharmaceutical composition, or method of this invention, wherein the ligand to the alpha-2-delta receptor, or a pharmaceutically acceptable salt thereof, is a compound of Formula A2DL I 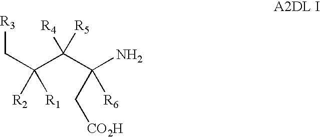
or a pharmaceutically acceptable salt thereof, wherein R₁ is hydrogen or (C₁-C₆)alkyl optionally substituted with from one to five fluorine atoms;

R₂ is hydrogen or (C₁-C₆)alkyl optionally substituted with from one to five fluorine atoms; or

R₁ and R₂, together with the carbon to which they are attached, form a three to six membered cycloalkyl ring;

R₃ is (C₁-C₆)alkyl, (C₃-C₆)cycloalkyl, (C₃-C₆)cycloalkyl-(C₁-C₃)alkyl, phenyl, phenyl-(C₁-C₃)alkyl, pyridyl, pyridyl-(C₁-C₃)alkyl, phenyl—NH(H)-, or pyridyl—NH(H)-, wherein each of the foregoing alkyl moieties can be optionally substituted with from one to five fluorine atoms, preferably with from zero to three fluorine atoms, and wherein said phenyl and said pyridyl and the phenyl and pyridyl moieties of said phenyl-(C₁-C₃)alkyl and said pyridyl-(C₁-C₃)alkyl, respectively, can be optionally substituted with from one to three substituents, preferably with from zero to two substituents, independently selected from chloro, fluoro, amino, nitro, cyano, (C₁-C₃)alkylamino, (C₁-C₃)alkyl optionally substituted with from one to three fluorine atoms and (C₁-C₃)alkoxy optionally substituted with from one to three fluorine atoms;

R₄ is hydrogen or (C₁-C₆)alkyl optionally substituted with from one to five fluorine atoms;

R₅ is hydrogen or (C₁-C₆)alkyl optionally substituted with from one to five fluorine atoms; and

R₆ is hydrogen or (C₁-C₆)alkyl.

Still another aspect of this invention is a combination, pharmaceutical composition, or method of this invention, wherein the ligand to the alpha-2-delta receptor is a compound of Formula A2DL I, or a pharmaceutically acceptable salt thereof, wherein

(i) R₁ is hydrogen and R₂ is not hydrogen; or

(ii) R₁ is methyl, R₂, R₄, R₅, and R6, are hydrogen, and R₃ is as defined above.

Still another aspect of this invention is a combination, pharmaceutical composition, or method of this invention, wherein the ligand to the alpha-2-delta receptor is a compound of Formula A2DL I, or a pharmaceutically acceptable salt thereof, wherein the ligand to the alpha-2-delta receptor is

(i) a compound of Formula A2DL IA, 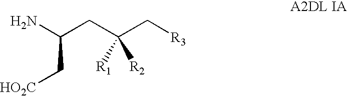
wherein R₁, R₂, and R₃ are defined as for Formula A2DL I above; or

(ii) a compound of Formula A2DL IA wherein R₁ is hydrogen, R₂ is methyl and R₃ is defined as for Formula A2DL I above; or

(iii) a compound of Formula A2DL IA selected from:

(3S,5R)-3-Amino-5-methyl-heptanoic acid;

(3S,5R)-3-Amino-5-methyl-octanoic acid; and

(3S,5R)-3-Amino-5-methyl-nonanoic acid; or

a pharmaceutically acceptable salt thereof; or

(iv) a compound of Formula A2DL I of the formula A2DL IB 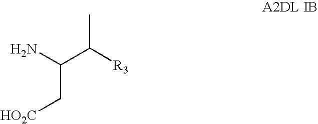
or a pharmaceutically acceptable salt thereof, wherein R₃ is defined as above; or

(v) a compound of Formula A2DL I of the formula A2DL IC 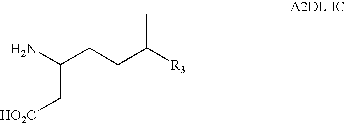
or a pharmaceutically acceptable salt thereof, wherein R₃ is defined as above for Formula A2DL I; or

(vi) a compound of Formula A2DL II 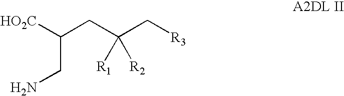
or a pharmaceutically acceptable salt thereof, wherein R₁, R₂, and R₃ are defined as for formula A2DL I above; or

(vii) a compound named 2-aminomethyl-4-propyl-heptanoic acid, or a pharmaceutically acceptable salt thereof; or

(viii) a compound of Formula A2DL II, or a pharmaceutically acceptable salt thereof, wherein R₁ is methyl, R₂ is hydrogen, and R₃ is as defined above for Formula A2DL I; or

(ix) a compound of Formula A2DL I of formula A2DL III 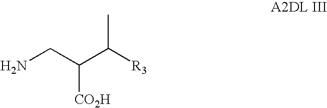
or a pharmaceutically acceptable salt thereof, wherein R₃ is defined as for Formula A2DL I above; or

(x) a compound of Formula A2DL I of formula A2DL IV 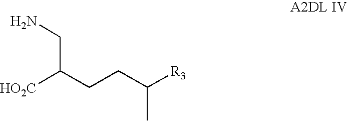
or a pharmaceutically acceptable salt thereof, wherein R₁ and R₃ are defined as for compounds of the Formula A2DL I above.

Still another aspect of this invention is a combination, pharmaceutical composition, or method of this invention, wherein the ligand to the alpha-2-delta receptor is a compound of Formula IC 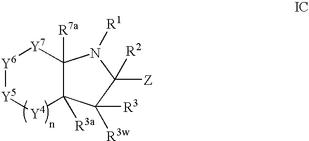
or a pharmaceutically acceptable salt thereof, wherein: Z is selected from COOH, C(O)N(H)R⁹, and Z¹; Z¹ is selected from: 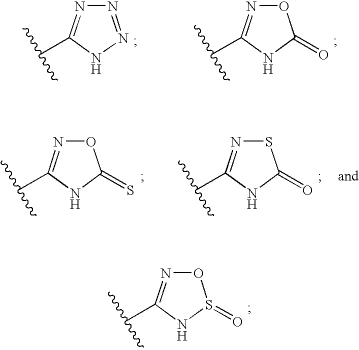
Each Y⁴, Y⁵, Y⁶, and Y⁷ is C(R¹⁰)R^{10w}; or One of Y⁴, Y⁵, Y⁶, and Y⁷ is selected from O, S, S(O), S(O)₂, and NR⁵, and the other three of Y⁴, Y⁵, Y⁶, and Y⁷ are each C(R¹⁰)R^{10w}; or
Two nonadjacent Y⁴, Y⁵, Y⁶ and Y⁷ are independently selected from O, S, S(O), S(O)₂, and NR⁵, and the other two of Y⁴, Y⁵, Y⁶, and Y7 are each C(R¹⁰)R^{10w};
Each R², R³,R^{3w}, R^{3a}, R^{7a}, R¹⁰, and R^{10w} is independently selected from: H, HO, H₂N, H₂NS(O)₂-(G)ₘ, HS, Halo, CN, CF₃, FC(H)₂_{O, F}₂C(H)O, CF₃O, and a group, which may be unsubstituted or substituted, independently selected from: C₁-C₆ alkyl-(G)ₘ-, C₂-C₆ alkenyl-(G)m-, C₂-C₆ alkynyl-(G)ₘ-, 2-to to 6-membered heteroalkyl-(G)ₘ-, 2-to 6-membered heteroalkenyl-(G)ₘ-, C₃-C₇ cycloalkyl-(G)ₘ-, C₃-C₇ cycloalkenyl-(G)ₘ-, C₇-C₁₀ bicycloalkyl-(G)ₘ-, 3-to 7-membered heterocycloalkyl-(G)ₘ-, 7-to 10-membered heterobicycloalkyl-(G)ₘ-, Phenyl-(G)ₘ-, Naphthyl-(G)ₘ-, 5-and 6-membered heteroaryl-(G)ₘ-, 8-to 10-membered heterobiaryl-(G)ₘ-, and any of the above R², R³, R^{3w}, R^{3a}, R^{7a}, R¹⁰, and R^{10w} groups each independently substituted on carbon or nitrogen atoms with from 1 to 6 substituents R^{X};
wherein R³ and R^{3w}, and any geminal pair of R¹⁰ and R^{10w}, and any two R^{X} substituents geminally substituted on a carbon atom in substituted R², R³, R^{3w}, R^{3a}, R^{7a}, R¹⁰, and R^{10w} groups further may independently be taken together with a carbon atom to which they are both bonded to form the group C(═O);
   Each R⁵ and R⁹ is independently H, HO, or a group, which may be unsubstituted or substituted, independently selected from: C₁-C₆ alkyl-(L)ₘ-, C₂-C₆ alkenyl-(L)ₘ-, C₂-C₆ alkynyl-(L)ₘ-, 2-to 6- membered heteroalkyl-(L)ₘ-, 2-to 6-membered heteroalkenyl-(L)ₘ-, C₃-C₇ cycloalkyl-(L)ₘ-, C₃-C₇ cycloalkenyl-(L)ₘ-, C₇-C₁₀ bicycloalkyl-(L)ₘ-, 3-to 7-membered heterocycloalkyl-(L)ₘ-, 7-to 10-membered heterobicycloalkyl-(L)ₘ-, Phenyl-(L)ₘ-, Naphthyl-(L)ₘ-, 5- and 6-membered heteroaryl-(L)ₘ-, 8-to 10-membered heterobiaryl-(L)ₘ-, and any of the above R⁵ and R⁹ groups independently substituted, on carbon or nitrogen atoms, with from 1 to 6 substituents R^{X};
R¹ is HO or a group that may be unsubstituted or substituted, independently selected from: C₁-C₆ alkyl-(T)ₘ-, C₂-C₆ alkenyl-(T)ₘ-, C₂-C₆ alkynyl-(T)ₘ-, 2-to 6-membered heteroalkyl-(T)ₘ-, 2-to 6-membered heteroalkenyl-(T)ₘ-, C₃-C₇ cycloalkyl-(T)ₘ-,C₃-C₇ cycloalkenyl-(T)ₘ-,C₇-C₁₀ bicycloalkyl-(T)ₘ-, 3-to 7-membered heterocycloalkyl-(T)ₘ-, 7-to 10-membered heterobicycloalkyl-(T)ₘ-, Phenyl-(T)ₘ-, Naphthyl-(T)ₘ-,5- and 6-membered heteroaryl-(T)ₘ-, 8-to 10-membered heterobiaryl-(T)ₘ-, and any of the above R¹ groups independently substituted on a carbon or nitrogen atom, with from 1 to 6 substituents R^{X};
R¹ may further be H when: (i) at least one of R², R³, R^{3w}, R^{3a}, R^{7a}, R¹⁰, and R^{10w} is not H, or (ii) Z is C(O)N(H)R⁹ wherein R⁹ is as defined above wherein m is 1 and L is S(O)₂, or (iv) Z is Z¹;
wherein any 2 groups each selected from R⁵, R¹⁰, and R^{10w} that are bonded to contiguous carbon or nitrogen atoms in Formula I may be taken together with the contiguous atoms in Formula I to which they are bonded to form C=C or C=N;
wherein any 2 groups selected from R¹, R², R³,R^{3w}, R^{3a}, R⁵, R^{7a}, R¹⁰, and R^{10w} that are bonded to contiguous carbon or nitrogen atoms in Formula I may be taken together to form (i) a CH₂ diradical, (ii) a 3-membered diradical selected from:

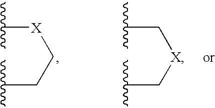

(iii) a 4-membered diradical selected from: 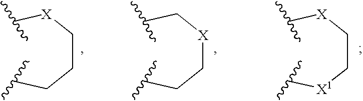
wherein any two groups R³ and R^{3w}, and R¹⁰ and R^{10w}, that are geminally bonded to a single carbon atom in Formula I may be taken together to form a 4-membered diradical as defined above or a 5-membered diradical selected from: 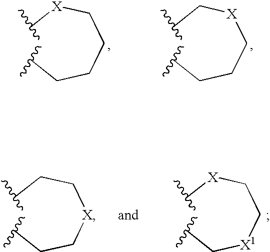
X is O, S, S(O), S(O)₂, or N-R;
X¹ is O or N-R;
Each G is independently selected from C(=O), S(O), S(O)₂, OC(O), N(R⁴)C(O), (C₁-C₈ alkylenyl)ₘ, (2-to 8-membered heteroalkylenyl)ₘ, and (C₁-C₈ alkylenyl)ₘ and (2-to 8-membered heteroalkylenyl)ₘ independently substituted on carbon or nitrogen atoms with from 1 to 4 substituents R^{X};
Each T is independently selected from S(O), S(O)₂, N(R⁴)C(O), (C₁-C₈ alkylenyl)ₘ, (2-to 8-membered heteroalkylenyl)ₘ, and (C₁-C₈ alkylenyl)ₘ and (2-to 8-membered heteroalkylenyl)ₘ independently substituted on carbon or nitrogen atoms with from 1 to 4 substituents R^{X};
Each L is independently selected from O, N(R⁴), S(O), S(O)₂, C(=O), C(O)O, OC(O), C(O)N(R⁴), N(R⁴)C(O), OC(O)N(R⁴), N(R⁴)C(O)O, N(R⁴)C(O)N(R^{4w}), (C₁-C₈ alkylenyl)ₘ, (2-to 8-membered heteroalkylenyl)ₘ, and (C₁-C₈ alkylenyl)ₘ and (2-to 8-membered heteroalkylenyl)ₘ independently substituted on carbon or nitrogen atoms with from 1 to 4 substituents R^{X};
Each R, R⁴, and R^{4w} is independently H or C₁-C₆ alkyl, which C₁-C₆ alkyl may be unsubstituted or substituted with from 1 to 3 substituents R^{X};
Each Rx is independently selected from: HO, H₂N, H₂NS(O)₂, CN, CF₃, FCH₂O, F₂C(H)O, CF₃O, O₂N, C₁-C₆ alkyl-(Q)ₘ-, 2-to 6-membered heteroalkyl-(Q)ₘ-, C₃-C₇ cycloalkyl-(Q)ₘ-, 3-to 7-membered heterocycloalkyl-(Q)ₘ-, Phenyl-(Q)ₘ, and 5-membered heteroaryl-(Q)ₘ,
   wherein phenyl and 5-membered heteroaryl-(Q)ₘ each is unsubstituted or independently substituted with from 1 to 3 substituents selected from halo, HO, HOC(O), CH₃OC(O), CH₃C(O), H₂N, CF₃, CN, and C₁-C₆ alkyl;
wherein each R^{X} substituent on a carbon atom may further be independently selected from: HS, (C₁-C₆ alkyl)-S, halo, and HO₂C; and
Each Q independently is O, N(R⁶), S(O), S(O)₂, C(=O), C(O)O, OC(O), C(O)N(R⁶), N(R⁶)C(O), OC(O)N(R⁶), N(R⁶)C(O)O, or N(R⁶)C(O)N(R^{6w});
Each R⁶ and R^{6w} independently is H or unsubstituted C₁-C₆ alkyl;
Each m independently is an integer of 0 or 1; and
Each n independently is an integer of from 0 to 2.

Still another aspect of this invention is a combination, pharmaceutical composition, or method of this invention, wherein the allosteric inhibitor of MMP-13 is a compound of Formula IC, or a pharmaceutically acceptable salt thereof, selected from any one of groups (i) to (v):

(i) 1 -methyl-octahydroindole-2-carboxylic acid;

[2(S), 3a(S), 7a(S)]-1-methyl-octahydro-indole-2-carboxylic acid hydrochloride;

[2(S), 3a(S), 7a(S)]-1-methyl-octahydro-indole-2-carboxylic acid hemi tartaric acid salt;

[2(S), 3a(S), 7a(S)]-1-methyl-octahydro-indole-2-carboxylic acid;

1-(2-amino-1-oxopropyl)-octahydro-indole-2-carboxylic acid;

[2(S), 3a(S), 7a(S)]-1-ethyl-octahydro-indole-2-carboxylic acid;

[2(R), 3a(R), 7a(R)]-1-methyl-octahydro-indole-2-carboxylic acid; or

(ii) (2R,3aS,7aS)-2-methyl-octahydroindole-2-carboxylic acid hydrochloride; and

(2S,3aS,7aS)-2-methyl-octahydroindole-2-carboxylic acid hydrochloride; or

(iii) 6-Ethyl-octahydro-indole-2-carboxylic acid hydrochloride;

(2S, 3aR, 6R/S, 7aR)-6-Phenyl-octahydro-indole-2-carboxylic acid;

6-Methoxy-octahydro-indole-2-carboxylic acid hydrochloride;

5-Ethyl-octahydro-indole-2-carboxylic acid hydrochloride;

5-Methyl-octahydro-indole-2-carboxylic acid hydrochloride;

5-Cyclohexylcarbonylamino-octahydro-indole-2-carboxylic acid hydrochloride;

5-Amino-octahydro-indole-2-carboxylic acid hydrochloride;

5-(1,1-Dimethylethyl)-octahydro-indole-2-carboxylic acid hydrochloride;

7-Methyl-octahydro-indole-2-carboxylic acid hydrochloride; and

4-Trifluoromethyl-octahydro-indole-2-carboxylic acid hydrochloride; or

(iv) (2S, 3aS, 7aS)-N-(Octahydroindole-2-carbonyl)-methanesulfonamide;

(2S, 3aS, 7aS)-N-(1-Methyl-octahydroindole-2-carbonyl)-methanesulfonamide;

(2S, 3aS, 7aS)-N-(Octahydroindole-2-carbonyl)-trifluoromethanesulfonamide; and

(2S, 3aS, 7aS)-N-(1-Methyl-octahydroindole-2-carbonyl)-trifluoromethanesulfonamide; or (v) (S,S,S)-3-(Octahydroindol-2-yl)-4H-[1 ,2,4]oxadiazol-5-one hydrochloride;

(S,S,S)-5-(Octahydroindol-2-yl)-1H-tetrazole.

(1aS, 1bS,5aS ,6aS)-octahydro-6-aza-cyclopropa[α]indene-6a-carboxylic acid; or

a pharmaceutically acceptable salt thereof.

Still another aspect of this invention is a combination, pharmaceutical composition, or method of this invention, wherein the ligand to the alpha-2-delta receptor is a compound of Formula ID

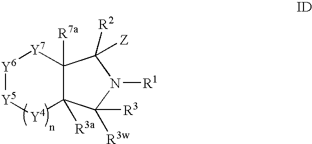

or a pharmaceutically acceptable salt thereof,

wherein:

Z is selected from COOH, C(O)N(H)R⁹, and Z¹;

Z¹ is selected from: 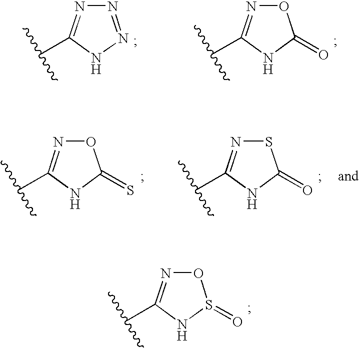
Each Y⁴, Y⁵, Y⁶, and Y⁷ is C(R¹⁰)R^{10w}; or
One of Y⁴, Y⁵, Y⁶, and Y⁷ is selected from O, S, S(O), S(O)₂, and NR⁵, and the other three of Y⁴, Y⁵, Y⁶, and Y⁷ are each C(R¹⁰)R^{10w}; or
Two nonadjacent Y4, Y⁵, Y⁶, and Y⁷ are independently selected from O, S, S(O), S(O)₂, and NR⁵, and the other two of Y⁴, Y⁵, Y⁶, and Y⁷ are each C(R¹⁰)R^{10w};
Each R², R³, R^{3w}, R^{3a}, R^{7a}, R¹⁰, and R^{10w} is independently selected from: H, HO, H₂N, H₂NS(O)₂-(G)ₘ, HS, Halo, CN, CF₃, FC(H)₂O, F₂C(H)O, CF₃O, and
a group, which may be unsubstituted or substituted, independently selected from:
C₁-C₆ alkyl-(G)ₘ-, C₂-C₆ alkenyl-(G)ₘ-, C₂-C₆ alkynyl-(G)ₘ-, 2-to 6-membered heteroalkyl-(G)ₘ-, 2-to 6-membered heteroalkenyl-(G)ₘ-, C₃-C₇ cycloalkyl-(G)ₘ-, C₃-C₇ cycloalkenyl-(G)ₘ-, C₇-C₁₀ bicycloalkyl-(G)ₘ-, 3-to 7-membered heterocycloalkyl-(G)ₘ-,7-to 10-membered heterobicycloalkyl-(G)ₘ-, Phenyl-(G)ₘ-, Naphthyl-(G)ₘ-, 5- and 6-membered heteroaryl-(G)ₘ-, 8-to 10-membered heterobiaryl-(G)ₘ-, and
any of the above R², R³, R^{3w}, R^{3a}, R^{7a}, R¹⁰, and R^{10w} groups each independently substituted on carbon or nitrogen atoms with from 1 to 6 substituents R^{X};
wherein R³ and R^{3w}, and any geminal pair of R¹⁰ and R^{10w}, and any two R^{X} substituents geminally substituted on a carbon atom in substituted R², R³, R^{3w}, R^{3a}, R^{7a}, R¹⁰, and R^{10w} groups further may independently be taken together with a carbon atom to which they are both bonded to form the group C(=O);
R¹ is HO or a group that may be unsubstituted or substituted, independently selected from:
C₁-C₆ alkyl-(T)ₘ-, C₂-C₆ alkenyl-(T)ₘ-, C₂-C₆ alkynyl-(T)ₘ-, 2-to 6-membered heteroalkyl-(T)ₘ-, 2-to 6-membered heteroalkenyl-(T)ₘ-C₃-C₇ cycloalkyl-(T)ₘ-7, C₃-C₇ cycloalkenyl-(T)ₘ-, C₇-C₁₀ bicycloalkyl-(T)ₘ-, 3-to 7-membered heterocycloalkyl-(T)ₘ-, 7-to 10-membered heterobicycloalkyl-(T)ₘ-, Phenyl-(T)ₘ-,Naphthyl-(T)ₘ-, 5- and 6-membered heteroaryl-(T)ₘ-, 8-to 10-membered heterobiaryl-(T)ₘ-, and
any of the above R¹ groups independently substituted on a carbon or nitrogen atom, with from 1 to 6 substituents R^{X};
R¹ may further be H when: (i) at least one of R², R³, R^{3w}, R^{3a}, R^{7a}, R¹⁰, and R^{10w} is not H, or (ii) Z is C(O)N(H)R⁹ wherein R⁹ is as defined above wherein m is 1 and L is S(O)₂, or (iv) Z is Z¹;
Each R⁵ and R⁹ is independently H, HO, or a group, which may be unsubstituted or substituted, independently selected from: C₁-C₆ alkyl-(L)ₘ-, C₂-C₆ alkenyl-(L)ₘ-, C₂-C₆ alkynyl-(L)ₘ-, 2-to 6-membered heteroalkyl-(L)ₘ-, 2-to 6-membered heteroalkenyl-(L)ₘ-, C₃-C₇ cycloalkyl-(L)ₘ-, C₃-C₇ cycloalkenyl-(L)ₘ-, C₇-C₁₀ bicycloalkyl-(L)ₘ-, 3-to 7-membered heterocycloalkyl-(L)ₘ-, 7-to 10-membered heterobicycloalkyl-(L)ₘ-, Phenyl-(L)ₘ-, Naphthyl-(L)ₘ-, 5- and 6-membered heteroaryl-(L)ₘ-, 8-to 10-membered heterobiaryl-(L)ₘ-, and
any of the above R⁵ and R⁹ groups independently substituted, on carbon or nitrogen atoms, with from 1 to 6 substituents R^{X};
wherein any 2 groups each selected from R⁵, R¹⁰, and R^{10w} that are bonded to contiguous carbon or nitrogen atoms in Formula I may be taken together with the contiguous atoms in Formula I to which they are bonded to form C=C or C=N;
wherein any 2 groups selected from R¹, R², R³, R^{3w}, R^{3a}, R⁵, R^{7a}, R¹⁰, and R^{10w} that are bonded to contiguous carbon or nitrogen atoms in Formula I may be taken together to form (i) a diradical selected from CH₂ and CH₂CH₂CH₂, (ii) a 3-membered diradical selected from:

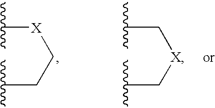

(iii) a 4-membered diradical selected from:

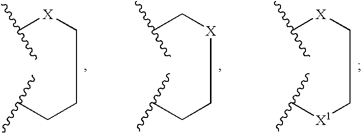

wherein any two groups R³ and R^{3w}, and R¹⁰ and R^{10w}, that are geminally bonded to a single carbon atom in Formula I may be taken together to form a 4-membered diradical as defined above or a 5-membered diradical selected from: 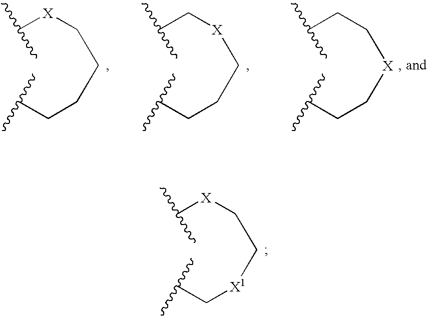
wherein any 2 groups selected from R¹, R², R³, R^{3w}, R^{3a}, R⁵, R^{7a}, R¹⁰, and R^{10w} that are bonded to noncontiguous carbon or nitrogen atoms in Formula I may be taken together to form (i) a CH₂CH₂ diradical or (ii) —O— diradical;
X is O, S, S(O), S(O)₂, or N-R;
X¹ is O or N-R;
Each G is independently selected from C(═O), S(O), S(O)₂, OC(O), N(R⁴)C(O), (C₁-C₈ alkylenyl)ₘ, (2-to 8-membered heteroalkylenyl)ₘ, and (C₁-C₈ alkylenyl)ₘ and (2-to 8-membered heteroalkylenyl)ₘ independently substituted on carbon or nitrogen atoms with from 1 to 4 substituents R^{X};
Each T is independently selected from S(O), S(O)₂, N(R⁴)C(O), (C₁-C₈ alkylenyl)ₘ, (2-to 8-membered heteroalkylenyl)ₘ, and (C₁-C₈ alkylenyl)ₘ and (2-to 8-membered heteroalkylenyl)ₘindependently substituted on carbon or nitrogen atoms with from 1 to 4 substituents R^{X};
Each L is independently selected from O, N(R⁴), S(O), S(O)₂, C(═O), C(O)O, OC(O), C(O)N(R⁴), N(R⁴)C(O), OC(O)N(R⁴), N(R⁴)C(O)O, N(R⁴)C(O)N(R⁴w), (C₁-C₈ alkylenyl)ₘ, (2-to 8-membered heteroalkylenyl)ₘ, and (C₁-C₈ alkylenyl)ₘ and (2- to 8-membered heteroalkylenyl)ₘ independently substituted on carbon or nitrogen atoms with from 1 to 4 substituents R^{X};
Each R, R⁴, and R^{4w} is independently H or C₁-C₆ alkyl, which C₁-C₆ alkyl may be unsubstituted or substituted with from 1 to 3 substituents R^{X};
Each R^{X} is independently selected from: HO, H₂N, H₂NS(O)₂, CN, CF₃, FCH₂O, F₂C(H)O, CF₃O, O₂N, C₁-C₆ alkyl-(Q)ₘ-, 2- to 6-membered heteroalkyl-(Q)ₘ-, C₃-C₇ cycloalkyl-(Q)ₘ-, 3- to 7-membered heterocycloalkyl-(Q)ₘ-, Phenyl-(Q)ₘ, and 5-membered heteroaryl-(Q)ₘ,
wherein phenyl and 5-membered heteroaryl-(Q)ₘ each is unsubstituted or independently substituted with from 1 to 3 substituents selected from halo, HO, HOC(O), CH₃O C(O), CH₃C(O), H₂N, CF₃, CN, and C₁-C₆ alkyl;
wherein each R^{X} substituent on a carbon atom may further be independently selected from: HS, (C₁-C₆ alkyl)—S, halo, and HO₂C; and
Each Q independently is O, N(R⁶), S(O), S(O)₂, C(═O), C(O)O, OC(O), C(O)N(R⁶), N(R⁶)C(O), OC(O)N(R⁶), N(R⁶)C(O)O, or N(R⁶)C(O)N(R^{6w});

Each R⁶ and R^{6w} independently is H or unsubstituted C₁-C₆ alkyl;

Each m independently is an integer of 0 or 1; and

Each n independently is an integer of from 0 to 2.

Still another aspect of this invention is a combination, pharmaceutical composition, or method of this invention, wherein the allosteric inhibitor of MMP-13 is a compound of Formula ID, or a pharmaceutically acceptable salt thereof, selected from any one of groups (i) to (iv):

(i) 1,2-dimethyl-octahydro-isoindole-1-carboxylic acid hydrochloride;

1-methyl-octahydro-isoindole-1-carboxylic acid hydrochloride;

5,6-Dimethoxy-octahydro-isoindole-1-carboxylic acid;

cis-2,3,3a,4,7,7a-Hexahydro-1H-isoindole-1-carboxylic acid hydrochloride;

diastereomer 1 of 6-chloro-2,2-dimethyl-octahydro-[1,3]dioxolo[4,5-ƒ]isoindole-5-carboxylic acid; and

diastereomer 2 of 6-chloro-2,2-dimethyl-octahydro-[1,3]dioxolo[4,5-ƒ]isoindole-5-carboxylic acid; or

a pharmaceutically acceptable salt thereof; or

(ii) 3-aza-6-oxabicyclo[4.3.0]nonane-2-carboxylic acid;

10-oxa-4-aza-tricyclo[5.2. 1.0^{2,6}]decane-3-carboxylic acid;

4-methyl-4aza-tricyclo[5.2.2.0^{2,6}]undecane-3-carboxylic acid hydrochloride; and

4-aza-tricyclo[5.2.2.0^{2,6}]undecane-3-carboxylic acid hydrochloride; or

a pharmaceutically acceptable salt thereof; or

(iii) 3-aza-6,6-difluorobicyclo[3.3.0]octane-2-carboxylic acid;

3-aza-6-fluorobicyclo[3.3.0]octane-2-carboxylic acid;

3-aza-6-n-butoxybicyclo [3.3.0]octane-2-carboxylic acid;

3-aza-hydroxybicyclo[3.3.0]octane-2-carboxylic acid hydrochloride;

3-aza-6-oxobicyclo[3.3.0]octane-2-carboxylic acid hydrochloride;

octahydro-pyrrolo[2,1-α]isoindole-9b-carboxylic acid hydrochloride; or

a pharmaceutically acceptable salt thereof; or

(iv) 7α-benzyl-octahydro-isoindole-1-carboxylic acid hydrochloride;

7α-methyl-octahydro-isoindole-1-carboxylic acid;

3,3-dimethyloctahydro-isoindole-1-carboxylic acid hydrochloride;

3-(octahydro-isoindol-1-yl-4H-[1,2,4]oxadiazol-5-one; and

3-(2-methyl-octahydro-isoindol-1-yl)4H-[1,2,4]oxadiazol-5-one; or

a pharmaceutically acceptable salt thereof.

Still another aspect of this invention is any one of the above combinations and pharmaceutical compositions, wherein the alpha-2-delta receptor ligand is an acyclic compound.

Still another aspect of this invention is any one of the above combinations, wherein the alpha-2-delta receptor ligand is a monocyclic compound wherein the monocyclic compound contains a cyclopentyl optionally substituted with 0, 1, or 2 methyl groups.

Still another aspect of this invention is any one of the above combinations and pharmaceutical compositions, wherein the alpha-2-delta receptor ligand is a monocyclic compound wherein the monocyclic compound contains a cyclohexyl substituted with 1 or 2 methyl groups.

Still another aspect of this invention is a combination, pharmaceutical composition, or method of this invention, wherein the allosteric inhibitor of MMP13, or a pharmaceutically acceptable salt thereof, is any one of the above named allosteric inhibitors of MMP-13, or a pharmaceutically acceptable salt thereof, and the ligand to the alpha-2-delta receptor, or a pharmaceutically acceptable salt thereof, is any one of the above named ligands to the alpha-2-delta receptor, or a pharmaceutically acceptable salt thereof.

Still another aspect of this invention is a combination, comprising a means for allosterically inhibiting MMP-13 and a means for binding to an alpha-2-delta receptor.

Still another aspect of this invention is a combination, comprising gabapentin, or a pharmaceutically acceptable salt thereof, and 4-{3-[2-(4-methoxy-benzyl)-2H-tetrazol-5-yl]-benzyloxymethyl}-benzoic acid, or a pharmaceutically acceptable salt thereof.

Still another aspect of this invention is a combination, comprising pregabalin, or a pharmaceutically acceptable salt thereof, and 4-(5-{3-[3-(4-fluoro-phenyl)-prop-1-ynyl]-phenyl}-tetrazol-2-ylmethyl)-benzoic acid, or a pharmaceutically acceptable salt thereof.

Still another aspect of this invention is a combination, comprising 3-(1-aminomethyl-cyclohexylmethyl)-4H-[1,2,4]oxadiazol-5-one, or a pharmaceutically acceptable salt thereof, and 4-(5-{3-[(pyridin-4-ylmethyl)-carbamoyl]-phenyl}-tetrazol-2-ylmethyl)-benzoic acid, or a pharmaceutically acceptable salt thereof.

Still another aspect of this invention is a combination, comprising C-[1-(1H-tetrazol-5-ylmethyl)-cycloheptyl]-methylamine, or a pharmaceutically acceptable salt thereof, and 3-[2-(4-methoxy-benzyl)-2H-tetrazol-5-yl]-N-(2-methoxy-pyridin-4-ylmethyl)-benzamide, or a pharmaceutically acceptable salt thereof.

Still another aspect of this invention is a combination, comprising (3S,5R)-3-aminomethyl-5-methyl-octanoic acid, or a pharmaceutically acceptable salt thereof, and 4-({3-[2-(4-fluoro-benzyl)-2H-tetrazol-5-yl]-benzoylamino}-methyl)-benzoic acid, or a pharmaceutically acceptable salt thereof.

Still another aspect of this invention is a combination, comprising (S,S)-(1-aminomethyl-3,4-dimethyl-cyclopentyl)-acetic acid, or a pharmaceutically acceptable salt thereof, and 4-[6-(3-phenyl-prop-1-ynyl)-4-oxo-4H-pyrido[3,4-d]pyrimidin-3-ylmethyl]-benzoic acid, or a pharmaceutically acceptable salt thereof.

Still another aspect of this invention is a combination, comprising [(lR,5R,6S)-6-(aminomethyl)bicyclo[3.2.0]hept-6-yl]acetic acid, or a pharmaceutically acceptable salt thereof, and 4-(5-{5-[3-(4-methoxy-phenyl)-prop-1-ynyl]-pyridin-3-yl}-tetrazol-2-ylmethyl)-benzoic acid, or a pharmaceutically acceptable salt thereof.

Still another aspect of this invention is a combination, comprising (1α,3α,5α)(3-amino-methyl-bicyclo[3.2.0]hept-3-yl)-acetic acid, or a pharmaceutically acceptable salt thereof, and 4-(5-{3-[3-(4-fluoro-phenyl)-prop-1-ynyl]-phenyl}-tetrazol-2-ylmethyl)-cyclohexanecarboxylic acid, or a pharmaceutically acceptable salt thereof.

Still another aspect of this invention is a combination, comprising (3S,5R)-3-amino-5-methyl-nonanoic acid, or a pharmaceutically acceptable salt thereof, and 4-[4-oxo-6-(3-phenyl-prop-1-ynyl)-4H-quinazolin-3-ylmethyl]-benzoic acid, or a pharmaceutically acceptable salt thereof.

Still another aspect of this invention is a combination, comprising (3S,5R)-3-amino-5-methyl-octanoic acid, or a pharmaceutically acceptable salt thereof, and 3-(4-methoxy-benzyl)-4-oxo-3,4-dihydro-quinazoline-6-carboxylic acid 4-methoxy-benzylamide, or a pharmaceutically acceptable salt thereof.

Still another aspect of this invention is a combination, comprising (3S,5R)-3-amino-5-methyl-heptanoic acid, or a pharmaceutically acceptable salt thereof, and 4-[6-(4-methoxy-benzylcarbamoyl)-4-oxo-1,4-dihydro-2H-quinazolin-3-ylmethyl]-benzoic acid, or a pharmaceutically acceptable salt thereof.

In one embodiment, this invention is a combination, comprising gabapentin and 4-{3-[2-(4-methoxy-benzyl)-2H-tetrazol-5-yl]-benzyloxymethyl}-benzoic acid.

In another embodiment, this invention is a combination, comprising pregabalin and 4-(5-{3-[3-(4-fluoro-phenyl)-prop-1-ynyl]-phenyl}-tetrazol-2-ylmethyl)-benzoic acid.

In another embodiment, this invention is a combination, comprising 3-(1-aminomethyl-cyclohexylmethyl)-4H-[1,2,4]oxadiazol-5-one hydrochloride and 4-(5-{3-[(pyridin-4-ylmethyl)-carbamoyl]-phenyl}-tetrazol-2-ylmethyl)-benzoic acid.

In another embodiment, this invention is a combination, comprising C-[1-(1H-tetrazol-5-ylmethyl)-cycloheptyl]-methylamine and 3-[2-(4-methoxy-benzyl)-2H-tetrazol-5-yl]-N-(2-methoxy-pyridin-4-ylmethyl)-benzamide.

In another embodiment, this invention is a combination, comprising (3S,5R)-3-aminomethyl-5-methyl-octanoic acid and 4-({3-[2-(4-fluoro-benzyl)-2H-tetrazol-5-yl]-benzoylamino}-methyl)-benzoic acid.

In another embodiment, this invention is a combination, comprising (S,S)-(1-aminomethyl-3,4-dimethyl-cyclopentyl)-acetic acid and 4-[6-(3-phenyl-prop-1-ynyl)-4-oxo-4H-pyrido[3,4-d]pyrimidin-3-ylmethyl]-benzoic acid.

In another embodiment, this invention is a combination, comprising [(lR,5R,6S)-6-(aminomethyl)bicyclo[3.2.0]hept-6-yl]acetic acid and 4-(5-{5-[3-(4-methoxy-phenyl)-prop-1-ynyl]-pyridin-3-yl}-tetrazol-2-ylmethyl)-benzoic acid.

In another embodiment, this invention is a combination, comprising (1α,3α,5α)(3-amino-methyl-bicyclo[3.2.0]hept-3-yl)-acetic acid and 4-(5-{3-[3-(4-fluoro-phenyl)-prop-1-ynyl]-phenyl}-tetrazol-2-ylmethyl)-cyclohexanecarboxylic acid.

In another embodiment, this invention is a combination, comprising (3S,5R)-3-amino-5-methyl-nonanoic acid and 4-[4-oxo-6-(3-phenyl-prop-1-ynyl)-4H-quinazolin-3-ylmethyl]-benzoic acid.

In another embodiment, this invention is a combination, comprising (3S,5R)-3-amino-5-methyl-octanoic acid and 3-(4-methoxy-benzyl)-4-oxo-3,4-dihydro-quinazoline-6-carboxylic acid 4-methoxy-benzylamide.

In another embodiment, this invention is a combination, comprising (3S,5R)-3-amino-5-methyl-heptanoic acid and 4-[6-(4-methoxy-benzylcarbamoyl)-4-oxo-1,4-dihydro-2H-quinazolin-3-ylmethyl]-benzoic acid.

Still another embodiment of this invention is a pharmaceutical composition, comprising any one of the above-recited combination embodiments, and a pharmaceutically acceptable carrier, diluent, or excipient.

Still another aspect of this invention is a pharmaceutical composition, comprising a combination comprising a means for allosterically inhibiting MMP-13 and a means for binding to an alpha-2-delta receptor, and a pharmaceutically acceptable carrier, diluent, or excipient.

Still another aspect of this invention is a method of treating an arthritic disease or disorder in a mammal, comprising administering to the mammal a therapeutically effective, sufficiently nontoxic amount of any one of the above combinations or pharmaceutical compositions of this invention, wherein the arthritic disease or disorder is selected from: osteoarthritis, rheumatoid arthritis, psoriatic arthritis, juvenile arthritis, reactive arthritis, Lyme arthritis, and infectious arthritis.

Still another aspect of this invention is a method of treating a joint disorder in a mammal, comprising administering to the mammal a therapeutically effective, sufficiently nontoxic amount of any one of the above combinations or pharmaceutical compositions of this invention, wherein the joint disorder is selected from: joint pain, joint inflammation, joint edema, and impaired joint function.

Still other aspects of this invention is the above method of treating joint pain, wherein the joint pain is acute joint pain, chronic joint pain, osteoarthritic joint pain, rheumatoid arthritic joint pain, post-operative joint pain, perioperative joint pain, or inflammatory joint pain.

Still another aspect of this invention is a method of treating joint cartilage damage in a mammal, comprising administering to the mammal a therapeutically effective, sufficiently nontoxic amount of any one of the above combinations or pharmaceutical compositions of this invention.

Still another aspect of this invention is a method of treating ankylosing spondylitis in a mammal, comprising administering to the mammal a therapeutically effective, sufficiently nontoxic amount of any one of the above combinations or pharmaceutical compositions of this invention.

Still another aspect of this invention is a method of treating fibromyalgia in a mammal, comprising administering to the mammal a therapeutically effective, sufficiently nontoxic amount of any one of the above combinations or pharmaceutical compositions of this invention.

Still another aspect of this invention is a method of treating fibromyalgic symptoms in a mammal, comprising administering to the mammal a therapeutically effective, sufficiently nontoxic amount of any one of the above combinations or pharmaceutical compositions of this invention, wherein the fibromyalgic symptoms are selected from fibromyalgic pain, sleep disturbance, and fatigue.

Still another aspect of this invention is a method of treating an inflammatory skin disease or disorder in a mammal, comprising administering to the mammal a therapeutically effective, sufficiently nontoxic amount of any one of the above combinations or pharmaceutical compositions of this invention, wherein the inflammatory skin disease or disorder is selected from: psoriasis, eczema, atopic dermatitis, contact dermatitis, discoid lupus, pemphigus vulgaris, bullous pemphigoid, and alopecia areata.

Still another aspect of this invention is a method of treating a skin ulcer or wound in a mammal, comprising administering to the mammal a therapeutically effective, sufficiently nontoxic amount of any one of the above combinations or pharmaceutical compositions of this invention.

Still another aspect of this invention is a method of promoting skin ulcer or wound healing and alleviating skin ulcer or wound pain in a mammal, comprising administering to the mammal a therapeutically effective, sufficiently nontoxic amount of any one of the above combinations or pharmaceutical compositions of this invention.

Still another aspect of this invention is a method of alleviating pain in a mammal, comprising administering to the mammal a therapeutically effective, sufficiently nontoxic amount of any one of the above combinations or pharmaceutical compositions of this invention, wherein the pain is selected from migraine, spinal pain, fibromyalgic pain, osteoarthritic pain, rheumatoid arthritic pain, and inflammatory pain.

Still another aspect of this invention is a method of alleviating neuropathic pain in a mammal, comprising administering to the mammal a therapeutically effective, sufficiently nontoxic amount of any one of the above combinations or pharmaceutical compositions of this invention, wherein the neuropathic pain includes post-herpetic neuralgia.

Still another aspect of this invention is a method of treating abdominal aortic aneurism in a mammal, comprising administering to the mammal a therapeutically effective, sufficiently nontoxic amount of any one of the above combinations or pharmaceutical compositions of this invention.

Still another aspect of this invention is a method of treating cancer in a mammal, comprising administering to the mammal a therapeutically effective, sufficiently nontoxic amount of any one of the above combinations or pharmaceutical compositions of this invention, wherein the cancer is selected from bone cancer, breast cancer, ovarian cancer, squamous carcinoma, head carcinoma, neck carcinoma, fibrosarcoma, chondrosarcoma, and basal cell carcinoma of the skin.

Still another aspect of this invention is a method of inhibiting cancer metastasis and alleviating cancer pain in a mammal, comprising administering to the mammal a therapeutically effective, sufficiently nontoxic amount of any one of the above combinations or pharmaceutical compositions of this invention, wherein the cancer is selected from bone cancer, breast cancer, ovarian cancer, squamous carcinoma, head carcinoma, neck carcinoma, fibrosarcoma, chondrosarcoma, and basal cell carcinoma of the skin.

Still another aspect of this invention is a method of treating a neurological disorder in a mammal, comprising administering to the mammal a therapeutically effective, sufficiently nontoxic amount of any one of the above combinations or pharmaceutical compositions of this invention, wherein the neurological disorder is selected from epilepsy and convulsions.

Still another aspect of this invention is a method of treating a neurological disorder in a mammal, comprising administering to the mammal a therapeutically effective, sufficiently nontoxic amount of any one of the above combinations or pharmaceutical compositions of this invention, wherein the neurological disorder is selected from social anxiety disorder, generalized anxiety disorder, and panic disorder.

Still another embodiment of this invention is any one of the above-recited methods of treating aspects, wherein what is administered to the mammal is any one of the above-recited combination embodiments.

Still other aspects of this invention include (i) a combination, comprising any single species selected from the allosteric inhibitors of MMP-13, or a pharmaceutically acceptable salt thereof, named above and any single species selected from the alpha-2-delta receptor ligands named above, or a pharmaceutically acceptable salt thereof; (ii) a pharmaceutical composition, comprising the combination described in (i) and a pharmaceutically acceptable carrier, diluent, or excipient; and (iii) any method of treating a disease or disorder described above using the pharmaceutical composition described in (ii).

Still another aspect of this invention is use of any one of the pharmaceutical compositions described herein in the preparation of a medicament to treat a mammalian disease or disorder in a mammal in need of treatment, wherein the disease or disorder is selected from those described above.

Still another aspect of this invention is use of any one of the above pharmaceutical compositions to treat a mammalian disease or disorder in a mammal in need of treatment, wherein the disease or disorder is selected from those described above.

Still another aspect of this invention is any one of Formulations 1-3 described below.

FORMULATION 1
Tablet Formulation:

| Ingredient | Amount (mg) |
|---|---|
| An allosteric inhibitor of | 25 |
| MMP-13 or an alpha-2-delta | |
| receptor ligand | |
| Lactose | 50 |
| Cornstarch (for mix) | 10 |
| Cornstarch (paste) | 10 |
| Magnesium stearate (1%) | 5 |
| Total | 100 |

The allosteric inhibitor of MMP-13 or the alpha-2-delta receptor ligand, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for inhibiting cartilage damage or treating osteoarthritis.

### Formulation 2

Injection Vials:

The pH of a solution of 500 g of an allosteric inhibitor of MMP-13, or a pharmaceutically acceptable salt thereof, or pregabalin, and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 25 mg of the allosteric inhibitor of MMP-13 or pregabalin.

The following Formulation 3 illustrates the invention pharmaceutical composition containing an invention combination in a single formulation with a pharmaceutically acceptable carrier, diluent, or excipient.

FORMULATION 3
Tablet Formulation:

| Ingredient | Amount (mg) |
|---|---|
| An allosteric inhibitor of MMP-13 | 25 |
| gabapentin | 20 |
| Lactose | 50 |
| Cornstarch (for mix) | 10 |
| Cornstarch (paste) | 10 |
| Magnesium stearate (1%) | 5 |
| Total | 120 |

The allosteric inhibitor of MMP-13, gabapentin, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of one of the above-listed diseases.

DETAILED DESCRIPTION OF THE INVENTION

As noted above, this invention relates to a combination of an allosteric inhibitor of matrix metalloproteinase-13 and a ligand to an alpha-2-delta receptor, a pharmaceutical composition comprising the combination, and a method of using the combination to treat a disease or disorder in a mammal suffering therefrom, wherein the disease or disorder is responsive to treatment in one aspect by an allosteric inhibitor of MMP-13 and in the same or a different aspect by a ligand to an alpha-2-delta receptor. An aspect of the disease or disorder being treated can include, for example, a sign such as altered gait, a symptom such as pain, inflammation, or edema, or a disease pathology such as damage to cartilage, bone, or extracellular matrix.

It should be appreciated that the combination of the present invention is not a single compound that is both an allosteric inhibitor of MMP-13 as defined herein and a ligand to an alpha-2-delta receptor as defined herein.

Allosteric inhibitors of MMP-13 useful in the present invention, their uses, and preparations thereof are described in U.S. patent application Nos. 10/071,032, 10/264,764; 10/269,197; and 10/417,073; and PCT International Patent Application Publication Nos. WO 02/064547; WO 02/064598; WO 02/064080; WO 02/064572; WO 02/064595; WO 02/064578; WO 02/064571; and WO 02/064568, and their corresponding U.S. patent application publication nos. US2002-0156061; US2003-0004172; US2003-0078276; US2002-0193377; US2002-0151558; US2002-0156069; US2002-0151555; and US2002-0161000, respectively, and PCT International Patent Application Publication Nos. WO 02/064599 and WO 03/032999, which correspond to U.S. application nos. 10/071,032 and 10/264,764, respectively.

Many alpha-2-delta ligands useful in the present invention have been described previously. For example, preparations of gabapentin, and pharmaceutically acceptable salts thereof, are described in U.S. Pat. No. 4,024,175 and its divisional U.S. Pat. No. 4,087,544. Preparations of pregabalin, and its pharmaceutically acceptable salts, are described in U.S. Pat. No. 5,563,175 and EP641330.

Other alpha-2-delta receptor ligands useful in the present invention include the alpha-2-delta receptor ligands, as well as structurally related homologs, stereoisomers, and regioisomers thereof, that are described in U.S. Pat. Nos. 5,563,175 and 6,518,289; U.S. Patent Application Nos. 60/462,850; 60/463,113; 10/324,929; and 10/401,060; PCT International Patent Application Publication Nos. WO 97/29101; WO 97/33858; WO 97/33859; WO 98/17627; WO 99/21824; WO 99/31057; WO 99/31074; WO 99/31075; WO 99/61424; WO 00/76958; WO 01/28978; WO 01/90052; WO 02/22568; WO 02/30871; and WO 02/85839; and European Patent Application Publication nos. EP 0,641,330; EP 1,178,034; and EP 1,201,240.

Still other alpha-2-delta receptor ligands useful in the present invention include the alpha-2-delta receptor ligands, as well as structurally related homologs, stereoisomers, and regioisomers thereof, that are described in PCT International Patent Application Publication Nos. WO 03/000642 (Nicox); WO 02/22568 (Grunenthal); WO 02/30871 (Grunenthal); WO 02/30881 (Grunenthal); WO 02/100392 (Xenoport); WO 02/100347 (Xenoport); WO 02/42414 (Xenoport); WO 02/32736 (Xenoport); and WO 02/28881 (Xenoport).

Additional aspects of this invention include, but are not limited to, those described below.

Some of the compounds utilized in an invention combination are capable of further forming pharmaceutically acceptable salts, including, but not limited to, acid addition and/or base salts. The acid addition salts are formed from basic compounds, whereas the base addition salts are formed from acidic compounds. All of these forms are within the scope of the compounds useful in the invention combination.

Pharmaceutically acceptable acid addition salts of the basic compounds useful in the invention combination include salts derived from inorganic acids such as hydrochloric, nitric, phosphoric, sulfuric, hydrobromic, hydroiodic, hydrofluoric, phosphorous, and the like, as well salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are salts of amino acids such as arginate and the like and gluconate, galacturonate (see, for example, Berge S. M. et al., “Pharmaceutical Salts,” J. of Pharma. Sci., 1977;66:1).

An acid addition salt of a basic compound useful in the invention combination is prepared by contacting the free base form of the compound with a sufficient amount of a desired acid to produce a salt in the conventional manner. The free base form of the compound may be regenerated by contacting the acid addition salt so formed with a base, and isolating the free base form of the compound in the conventional manner.

A pharmaceutically acceptable base addition salt of an acidic compound useful in the invention combination may be prepared by contacting the free acid form of the compound with a metal cation such as an alkali or alkaline earth metal cation, or an amine, especially an organic amine. Examples of suitable metal cations include sodium cation (Na⁺), potassium cation (K⁺), magnesium cation (Mg²⁺), calcium cation (Ca²⁺), and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, supra., 1977).

A base addition salt of an acidic compound useful in the invention combination may be prepared by contacting the free acid form of the compound with a sufficient amount of a desired base to produce the salt in the conventional manner. The free acid form of the compound may be regenerated by contacting the salt form so formed with an acid, and isolating the free acid of the compound in the conventional manner.

The free base forms or free acid forms of the compounds useful in the invention combination differ from their respective salt forms somewhat in certain physical properties such as solubility, crystal structure, hygroscopicity, and the like, but otherwise the salts are equivalent to their respective free base or acid forms for purposes of the present invention.

Certain of the compounds useful in the invention combination can exist in unsolvated forms as well as solvated forms, including hydrated forms. In general, the solvated forms, including hydrated forms, are equivalent to unsolvated forms and are encompassed within the scope of the present invention.

Certain of the compounds useful in the invention combination possess one or more chiral centers, and each center may exist in the (R) or (S) configuration. An invention combination may utilize any diastereomeric, enantiomeric, or epimeric form of a compound useful in the invention combination, as well as mixtures thereof.

Additionally, certain compounds useful in the invention combination may exist as geometric isomers such as the entgegen (E) and zusammen (Z) isomers of 1,2-disubstituted alkenyl groups or cis and trans isomers of disubstituted cyclic groups. An invention combination may utilize any cis, trans, syn, anti, (E), or (Z) isomer of a compound useful in the invention combination, as well as mixtures thereof.

Certain compounds useful in the invention combination can exist as two or more tautomeric forms. Tautomeric forms of the compounds may interchange, for example, via enolization/de-enolization, 1,2-hydride, 1,3-hydride, or 1,4-hydride shifts, and the like. An invention combination may utilize any tautomeric form of a compound useful in the invention combination, as well as mixtures thereof.

Other mammalian diseases and disorders which are treatable by administration of an invention combination or a further combination with an NSAID include: fever (including rheumatic fever and fever associated with influenza and other viral infections), common cold, dysmenorrhea, menstrual cramps, inflammatory bowel disease, Crohn's disease, emphysema, acute respiratory distress syndrome, asthma, bronchitis, chronic obstructive pulmonary disease, Alzheimer's disease, organ transplant toxicity, cachexia, allergic reactions, allergic contact hypersensitivity, cancer (such as solid tumor cancer including colon cancer, breast cancer, lung cancer and prostrate cancer; hematopoietic malignancies including leukemias and lymphomas; Hodgkin's disease; aplastic anemia, skin cancer and familiar adenomatous polyposis), tissue ulceration, peptic ulcers, gastritis, regional enteritis, ulcerative colitis, diverticulitis, recurrent gastrointestinal lesion, gastrointestinal bleeding, coagulation, anemia, synovitis, gout, ankylosing spondylitis, restenosis, periodontal disease, epidermolysis bullosa, osteoporosis, loosening of artificial joint implants, atherosclerosis (including atherosclerotic plaque rupture), aortic aneurysm (including abdominal aortic aneurysm and brain aortic aneurysm), periarteritis nodosa, congestive heart failure, myocardial infarction, stroke, cerebral ischemia, head trauma, spinal cord injury, neuralgia, neuro-degenerative disorders (acute and chronic), autoimmune disorders, Huntington's disease, Parkinson's disease, migraine, depression, peripheral neuropathy, pain (including low back and neck pain, headache and toothache), gingivitis, cerebral amyloid angiopathy, nootropic or cognition enhancement, amyotrophic lateral sclerosis, multiple sclerosis, ocular angiogenesis, comeal injury, macular degeneration, conjunctivitis, abnormal wound healing, muscle or joint sprains or strains, tendonitis, skin disorders (such as psoriasis, eczema, scleroderma and dermatitis), myasthenia gravis, polymyositis, myositis, bursitis, burns, diabetes (including types I and II diabetes, diabetic retinopathy, neuropathy and nephropathy), tumor invasion, tumor growth, tumor metastasis, corneal scarring, scleritis, immunodeficiency diseases (such as AIDS in humans and FLV, FIV in cats), sepsis, premature labor, hypoprothrombinemia, hemophilia, thyroiditis, sarcoidosis, Behcet's syndrome, hypersensitivity, kidney disease, Rickettsial infections (such as Lyme disease, Erlichiosis), Protozoan diseases (such as malaria, giardia, coccidia), reproductive disorders (preferably in livestock), epilepsy, convulsions, and septic shock.

The invention method is useful in human and veterinary medicines for treating mammals suffering from one or more of the diseases and disorders recited herein.

One of ordinary skill in the art will appreciate that when using the combinations of the invention in the treatment of a specific disease that the combinations of the invention may be combined with various existing therapeutic agents used for that disease.

It should be appreciated that the combination of the present invention may itself be used in combination with an NSAID for treating arthritic pain, cartilage damage, inflammation, and the like.

For the treatment of rheumatoid arthritis, the combinations of the invention may be combined with agents such as TNF-α inhibitors such as anti-TNF monoclonal antibodies and TNF receptor immunoglobulin molecules (such as Enbrel®), low dose methotrexate, lefunimide, hydroxychloroquine, d-penicillamine, auranofin or parenteral or oral gold.

The combinations of the invention can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib and rofecoxib, analgesics and intraarticular therapies such as corticosteroids and hyaluronic acids such as hyalgan and synvisc.

This invention also relates to a method of or a pharmaceutical composition for treating inflammatory processes and diseases comprising administering a combination of this invention to a mammal, including a human, cat, livestock or dog, wherein said inflammatory processes and diseases are defined as above and said inhibitory combination is used in combination with one or more other therapeutically active agents under the following conditions:

A.) where a joint has become seriously inflamed as well as infected at the same time by bacteria, fungi, protozoa and/or virus, said inhibitory combination is administered in combination with one or more antibiotic, antifungal, antiprotozoal and/or antiviral therapeutic agents;

B.) where a multi-fold treatment of pain and inflammation is desired, said inhibitory combination is administered in combination with inhibitors of other mediators of inflammation, comprising one or more members independently selected from the group consisting essentially of:

(1) NSAIDs;

(2) H₁-receptor antagonists;

(3) kinin-B₁- and B₂ -receptor antagonists;

(4) prostaglandin inhibitors selected from the group consisting of PGD-, PGF- PGI₂ - and PGE-receptor antagonists;

(5) thromboxane A₂ (TXA₂-) inhibitors;

(6) 5-, 12- and 15-lipoxygenase inhibitors;

(7) leukotriene LTC₄ -, LTD4/LTE₄ - and LTB₄ -inhibitors;

(8) PAF-receptor antagonists;

(9) gold in the form of an aurothio group together with one or more hydrophilic groups;

(10) immunosuppressive agents selected from the group consisting of cyclosporine, azathioprine and methotrexate;

(11) anti-inflammatory glucocorticoids;

(12) penicillamine;

(13) hydroxychloroquine;

(14) anti-gout agents including colchicine; xanthine oxidase inhibitors including allopurinol; and uricosuric agents selected from probenecid, sulfinpyrazone and benzbromarone;

C. where older mammals are being treated for disease conditions, syndromes and symptoms found in geriatric mammals, said inhibitory combination is administered in combination with one or more members independently selected from the group consisting essentially of:

(1) cognitive therapeutics to counteract memory loss and impairment;

(2) anti-hypertensives and other cardiovascular drugs intended to offset the consequences of atherosclerosis, hypertension, myocardial ischemia, angina, congestive heart failure and myocardial infarction, selected from the group consisting of:

a. diuretics;

b. vasodilators;

c. β-adrenergic receptor antagonists;

d. angiotensin-II converting enzyme inhibitors (ACE-inhibitors), alone or optionally together with neutral endopeptidase inhibitors;

e. angiotensin II receptor antagonists;

f. renin inhibitors;

g. calcium channel blockers;

h. sympatholytic agents;

i. α₂-adrenergic agonists;

j. α-adrenergic receptor antagonists; and

k. HMG-CoA-reductase inhibitors (anti-hypercholesterolemics);

(3) antineoplastic agents selected from:

a. antimitotic drugs selected from:

i. vinca alkaloids selected from:

[1] vinblastine and [2] vincristine;

(4) growth hormone secretagogues;

(5) strong analgesics;

(6) local and systemic anesthetics; and

(7) H₂ -receptor antagonists, proton pump inhibitors and other gastroprotective agents.

The active ingredient of the present invention may be administered in combination with inhibitors of other mediators of inflammation, comprising one or more members selected from the group consisting essentially of the classes of such inhibitors and examples thereof which include, matrix metalloproteinase inhibitors, aggrecanase inhibitors, TACE inhibitors, leucotriene receptor antagonists, IL-1 processing and release inhibitors, ILra, H₁ -receptor antagonists; kinin-B₁ - and B₂ -receptor antagonists; prostaglandin inhibitors such as PGD-, PGF- PGI₂ - and PGE-receptor antagonists; thromboxane A₂ (TXA2-) inhibitors; 5- and 12-lipoxygenase inhibitors; leukotriene LTC₄ -, LTD₄/LTE₄ - and LTB₄ -inhibitors; PAF-receptor antagonists; gold in the form of an aurothio group together with various hydrophilic groups; immunosuppressive agents, e.g., cyclosporine, azathioprine and methotrexate; anti-inflammatory glucocorticoids; penicillamine; hydroxychloroquine; anti-gout agents, e.g., colchicine, xanthine oxidase inhibitors, e.g., allopurinol and uricosuric agents, e.g., probenecid, sulfinpyrazone and benzbromarone.

The combinations of the present invention may also be used in combination with anticancer agents such as endostatin and angiostatin or cytotoxic drugs such as adriamycin, daunomycin, cis-platinum, etoposide, taxol, taxotere and alkaloids, such as vincristine and antimetabolites such as methotrexate.

The combinations of the present invention may also be used in combination with anti-hypertensives and other cardiovascular drugs intended to offset the consequences of atherosclerosis, including hypertension, myocardial ischemia including angina, congestive heart failure and myocardial infarction, selected from vasodilators such as hydralazine, β-adrenergic receptor antagonists such as propranolol, calcium channel blockers such as nifedipine, α₂-adrenergic agonists such as clonidine, α-adrenergic receptor antagonists such as prazosin and HMG-CoA-reductase inhibitors (anti-hypercholesterolemics) such as lovastatin or atorvastatin.

The combination of the present invention may also be administered in combination with one or more antibiotic, antifungal, antiprotozoal, antiviral or similar therapeutic agents.

The combinations of the present invention may also be used in combination with CNS agents such as antidepressants (such as sertraline), anti-Parkinsonian drugs (such as L-dopa, requip, mirapex, MAOB inhibitors such as selegine and rasagiline, comP inhibitors such as Tasmar, A-2 inhibitors, dopamine reuptake inhibitors, NMDA antagonists, nicotine agonists, dopamine agonists and inhibitors of neuronal nitric oxide synthase) and anti-Alzheimer's drugs such as donepezil, tacrine, COX-2 inhibitors, propentofylline or metryfonate.

The combinations of the present invention may also be used in combination with osteoporosis agents such as roloxifene, lasofoxifene, droloxifene or fosomax and immunosuppressant agents such as FK-506 and rapamycin.

The combinations of this invention may also be used in combination with a therapeutic biological agent such as CP-870.

The present invention also relates to the formulation of the combination of the present invention alone or with one or more other therapeutic agents which are to form the intended combination, including wherein the therapeutically active agents have varying half-lives, by creating controlled-release forms of said agents with different release times which achieves relatively uniform dosing; or, in the case of non-human patients, a medicated feed dosage form in which said agents used in the combination are present together in admixture in the feed composition.

There is further provided in accordance with the present invention co-administration in which the combination of drugs is achieved by the simultaneous administration of said drugs to be given in combination; including co-administration by means of different dosage forms and routes of administration; the use of combinations in accordance with different but regular and continuous dosing schedules whereby desired plasma levels of said drugs involved are maintained in the patient being treated, even though the individual drugs making up said combination are not being administered to said patient simultaneously.

Definitions:

It should be appreciated that the beneficial pharmacological and veterinary activities provided by the alpha-2-delta receptor ligands utilized by the combinations, pharmaceutical compositions, and methods of this invention are not necessarily a result of the alpha-2-delta receptor binding activities of these compounds. Alpha-2-delta receptors may, or may not, mediate the biological pathway(s) by which active compounds of this invention identified as ligands to an alpha-2-delta receptor produce their biological effects. The pharmacological and veterinary utilities of these compounds thus should not be limited to diseases or disorders responsive to liganding of an alpha-2-delta receptor.

The phrase “allosteric inhibitor of MMP-13” means an allosteric-binding inhibitor of MMP-13 with an IC₅₀ with MMP-13 full length enzyme or a catalytic domain thereof of less than, or equal to, 5 μM, but is not substantially bound to, coordinated to, or ligated to, either directly, or indirectly via a bridging water molecule, the catalytic zinc cation of a MMP-13 during inhibition. The catalytic zinc cation of the MMP-13 enzyme binds, ligates, or coordinates a functional group (e.g., an amide carbonyl oxygen atom) of a natural substrate(s).

Allosteric inhibitors of MMP-13 may be identified according to the method of Biological Method 3 below. As used herein, an allosteric inhibitor of MMP-13 is a noncompetitive or uncompetitive inhibitor of MMP-13 as characterized by Biological Method 3, whereas a non-allosteric inhibitor of MMP-13 is a competitive inhibitor of MMP-13 as characterized by Biological Method 3.

It should be appreciated that inhibitors of MMP-13 that are not allosteric inhibitors thereof typically are compounds that simultaneously bind to a catalytic zinc cation of the MMP-13 and occupy a nearby binding pocket (e.g., the S1′ pocket) of the MMP-13 at a binding energy minimum. A binding energy minimum is an inhibitor-enzyme form such as for a co-crystallized inhibitor bound MMP-13. Such a non-allosterically binding compound, when it is bound to an MMP-13, has a coordinatively positioned functional group that is bound to the catalytic zinc cation of the MMP-13 directly or via a bridging water molecule. This coordinatively positioned functional group is typically COOH, SH, C(O)N(H)OH, or a group such as the cyclic diradical of formula (A)

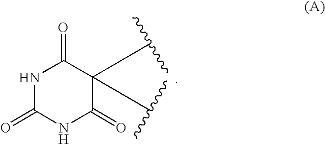

It should further be appreciated that allosteric inhibitors of MMP-13 can have a functional group such as COOH, SH, C(O)N(H)OH, or the diradical of formula (A) provided that the allosteric inhibitors of MMP-13 do not successfully present such a functional group in an orientation that is both favorable for binding to the catalytic zinc cation of the MMP-13 and for simultaneously occupying at least one nearby binding pocket of the MMP-13 enzyme at a binding energy minimum. Allosteric inhibitors of MMP-13 with such functional groups instead bind to a site removed from the catalytic zinc cation of MMP-13 (i.e., at an allosteric site) at a binding energy minimum.

A MMP-13 inhibitor can readily be identified as either an allosteric inhibitor of MMP-13 or a non-allosteric inhibitor of MMP-13 at binding energy minimum by either co-crystallization of the compound with MMP-13 catalytic domain according to conventional methods and observing the binding mode by x-ray diffraction techniques or by characterizing the inhibition of a particular compound as either competitive or noncompetitive or uncompetitive according to the method of Biological Method 3 below.

In another aspect of this invention, an allosteric inhibitor of MMP-13, as used in the present invention, is as defined above and further is a compound that is ≧5 times more potent in vitro versus MMP-13, or a truncated form thereof, than versus at least 2 other matrix metalloproteinase enzymes, including MMP-1, MMP-2, MMP-3, MMP-7, MMP-8, MMP-9, MMP-10, MMP-11, MMP-12, MMP-14, MMP-17, MMP-18, MMP-19, MMP-21, and MMP-26, and tumor necrosis factor alpha convertase (“TACE”). Another aspect of the present invention is a combination comprising an allosteric inhibitor of MMP-13 that is a selective inhibitor of MMP-13 over MMP-1.

Other aspects of the present invention are allosteric inhibitors of MMP-13, or a pharmaceutically acceptable salt thereof, that are ≧10, ≧20, ≧50, ≧100, or ≧1000 times more potent versus MMP-13 than versus at least two of any other MMP enzyme or TACE.

Still other aspects of the present invention are allosteric inhibitors of MMP-13, or a pharmaceutically acceptable salt thereof, that are ≧10, ≧20, ≧50, ≧100, or ≧1000 times more potent versus MMP-13 than versus 2, 3, 4, 5, 6, 7, or 8 other MMP enzymes, or versus TACE and 1, 2, 3, 4, 5, 6, 7, or 8 other MMP enzymes.

It should be appreciated that the phrase “alpha-2-delta receptor ligand” is synonymous with the phrase “alpha-2-delta ligand” and includes a compound that bind to an alpha-2-delta receptor with an IC₅₀ of less than, or equal to, 10 μM, as well as structurally related homologs, stereoisomers, and regioisomers thereof that do not bind to an alpha-2-delta receptor with an IC₅₀ of less than, or equal to, 10 μM, but nevertheless are efficacious in an MIA animal model of osteoarthritic pain with an ED₄₀ of less than, or equal to, 100 mg of the alpha-2-delta receptor ligand per kilogram of test animal body weight (≦100 mg/kg). Alpha-2-delta receptor ligands may be identified according to the method of Biological Method 5 below. Alpha-2-delta receptor ligands efficacious in an MIA animal model of osteoarthritic pain with an ED₄₀ of ≦100 mg/kg may be identified according to Biological Method 6 below.

The phrase “therapeutic biological agent” includes CP-870, etanercept (a tumor necrosis factor alpha (“TNF-alpha”) receptor immunoglobulin molecule; trade names ENBREL® and ENBREL ENTANERCEPT® by Immunex Corporation, Seattle, Wash.), infliximab (an anti-TNF-alpha chimeric IgG 1K monoclonal antibody; tradename REMICADE® by Centocor, Inc., Malvern, Pa.), methotrexate (tradename RHEUMATREX® by American Cyanamid Company, Wayne, N.J.), and adalimumab (a human monoclonal anti-TNF-alpha antibody; tradename HUMIRA® by Abbott Laboratories, Abbott Park, Ill.).

It should be appreciated that the terms “uses”, “utilizes”, and “employs”, and their derivatives thereof, are used interchangeably when describing an aspect of an invention method, composition, or combination.

The terms “including,” “having,” and “containing” are open ended unless otherwise indicated.

The term “patient” means a mammal, and the two terms are used interchangeably herein.

The terms “binding,” “coordinating,” and liganding” are synonymous and refer to an act of connecting directly via a noncovalent or covalent bond, or indirectly via a bridging water molecule, to a target molecule, including an enzyme or a receptor.

The term “ligand” means a compound that is capable of liganding as defined herein.

The term “treating” means administration, according to the invention method as defined above, of a combination or pharmaceutical composition of this invention that eliminates, alleviates, inhibits or prevents the onset of, inhibits the progress of, prevents further progress of, or reverses progression of, in part or in whole, any one or more of the pathological hallmarks or symptoms of any one of the diseases and disorders being treated, including, but not limited to, the pathological hallmark of cartilage damage and the symptoms of pain and inflammation. A patient at risk for developing a disease or disorder may, or may not, be prophylactically treated just as a patient having the disease or disorder may be medically treated.

The term “preventing” means prophylactic administration, according to the invention method as defined above, of a combination or pharmaceutical composition of this invention to an asymptomatic patient at risk for the disease or disorder being prevented to inhibit the onset of an associated pathological hallmark or symptom, including, but not limited to, the pathological hallmark of cartilage damage and the symptoms of pain and inflammation. Further, once onset of a pathological hallmark or symptom has begun, preventing means to prevent further progression or reverse progression, in part or in whole, of the pathological hallmark or symptom.

The term “inhibiting” means an act of preventing, suppressing, slowing, reducing, eliminating, or reversing a biological activity or effect of a target molecule, including an enzyme or a receptor, that is being inhibited.

The term “IC₅₀” means the concentration of a compound, usually expressed as micromolar or nanomolar, required to inhibit an enzyme's catalytic activity by 50%.

The term “ED₄₀” means the dose in milligrams of a compound per kilogram of patient body weight required to treat a disease with a statistically significant effect in about 40% of a patient group.

The term “ED₃₀” means the dose in milligrams of compound per kilogram of patient body weight required to treat a disease with a statistically significant effect in 30% of a patient group.

The phrase “pharmaceutical composition” means a composition suitable for administration in medical or veterinary use.

The term “admixed” and the phrase “in admixture” are synonymous and mean in a state of being in a homogeneous or heterogeneous mixture. In another aspect of this invention, the pharmaceutical composition is a homogeneous mixture.

It should be appreciated that an allosteric inhibitor of MMP-13 and an alpha-2-delta receptor ligand may be formulated together in the same pharmaceutical composition or may be formulated separately in different pharmaceutical compositions, the pharmaceutical compositions may be administered simultaneously (temporally overlapping administration) or sequentially (non-temporally overlapping administration provided efficacious blood levels of each active component temporally overlap), in the same form (e.g., both in tablets) or in different forms (e.g., one a tablet and one as a solution for intravenous administration), via the same route (e.g., both oral) or by different routes (e.g., one oral and one intravenous injection).

It should be appreciated that an invention combination or pharmaceutical composition may be administered in an amount that is “sufficiently nontoxic.” A sufficiently nontoxic amount may be an efficacious dose which may potentially produce toxic symptoms in certain patients at certain doses, but because of the pernicious nature of the disease being treated or the idiosyncratic nature of the appearance of the toxic symptoms in a patient population, and the risk/benefit value to the patient or patient population of the invention combination being used, it is acceptable to patients, medical or veterinary practitioners, and drug regulatory authorities to use such a sufficiently nontoxic dose. Under certain circumstances, a sufficiently nontoxic dose may be an efficacious dose at which more than 10% of a patient population experience one or more toxic symptoms but wherein the disease being treated is a life-threatening disease such as cancer, including breast cancer, and there are no curative treatment options. Alternatively, a sufficiently nontoxic dose may be a generally nontoxic efficacious dose at which a certain majority of patients being treated do not experience drug-related toxicity, although a small percentage of the patient population may be susceptible to an idiosyncratic toxic effect at the dose.

For the purposes of this invention, the term “arthritis” includes osteoarthritis, rheumatoid arthritis, degenerative joint disease, spondyloarthropathies, gouty arthritis, systemic lupus erythematosus, juvenile arthritis, and psoriatic arthritis. A combination or pharmaceutical composition of this invention may also be useful for treating degenerative joint disease, spondyloarthropathies, gouty arthritis, systemic lupus erythematosus, juvenile arthritis, and psoriatic arthritis.

The phrase “cartilage damage” means a disorder of articular cartilage and subchondral bone characterized by hypertrophy of tissues in and around an involved joint, which may or may not be accompanied by deterioration of articular cartilage surface. As used herein, the phrase cartilage damage relates to damage to joint cartilage or cartilage of the spinal column. In one aspect, cartilage damage relates to damage to joint cartilage.

It should be appreciated that cartilage is a multicellular tissue found at joint linings and in other parts of the body, including the nose, for example. Cartilage tissue provides frictionless surfaces for joint movement, and structure and support for soft tissue features of the body such as the nostrils of the nose. When cartilage tissue is damaged by disease or trauma, breakdown products are formed and the physiological function of the tissue is impaired. There are principally three types of cartilage in a body, including articular cartilage.

The phrase “inhibiting cartilage damage” means the therapeutic effect of a combination as described above, that eliminates, alleviates, inhibits or prevents the onset of, inhibits the progress of, prevents further progress of, or reverses progression of, in part or in whole, any one or more pathological hallmarks or symptoms of cartilage damage observed for any of the diseases and disorders which have cartilage damage as a component of the disease or disorder pathology. A patient at risk for developing cartilage damage may be prophylactically treated just as a patient having cartilage damage may be medically treated.

It should be appreciated that a pathological hallmark of a disease or disorder relates to a structural change in a body that is a direct or indirect result of the body being afflicted with the disease or disorder. Such structural changes may be identified by clinical observation, examination of biopsied tissue, pathological examination or by imaging techniques such as X-ray or magnetic resonance imaging, of the affected structure. Illustrative examples of a pathological hallmark include histopathological damage to cartilage, thickening or thinning of bone, hypertrophy of muscle, fibrosis, a tear in a ligament or tendon, damage to a nerve cell covering, and the like.

The term “osteoarthritis” includes diseases of the joint principally characterized by the pathological hallmark of joint cartilage damage, and optionally the symptom of joint pain. Osteoarthritis patients typically do not suffer from inflammation of the joint, although they may experience transient inflammatory flares from time to time.

The term “rheumatoid arthritis” includes rheumatic diseases of the joint principally characterized by the symptom of joint inflammation, and optionally joint pain. Rheumatoid arthritis patients may also experience damage to joint cartilage or bone.

The phrase “skin ulcer” means a break in the skin due to a wound, surgical incision, bed sore, or the like that may, or may not, heal (close) without surgical or pharmacological intervention within 1 month.

An invention combination or pharmaceutical composition may be administered prophylactically to prevent or inhibit the onset of osteoarthritis, rheumatoid arthritis, loss of joint function, cartilage damage, or any pain in an asymptomatic patient (mammal). It should be appreciated that an asymptomatic patient at risk for the disease or disorder being prevented may be identified by analysis of genetic risk factors (inherited or spontaneous mutation diseases and disorders), family medical history, occupation, participation in athletic activities, general medical screening, and the like.

The term “improving” means eliminating or preventing the loss, inhibiting further loss, or improving, in part or in whole, of any one or more of the clinical measures of a function in a patient suffering from any one of the diseases and disorders being improved, including, but not limited rheumatoid arthritis and osteoarthritis.

The phrase “joint function” relates to any one or more of the clinical assessments of joint function, including stiffness, range of movement, flexibility, and movement-related symptoms (e.g., altered gait, pain, warmth, or inflammation), in a patient suffering from any one of the diseases and disorders being improved, including, but not limited the diseases of rheumatoid arthritis and osteoarthritis. A clinician may use the Western Ontario and McMaster Universities Osteoarthritis Index (“WOMAC”) to assess joint function.

The phrase “joint pain” means any pain in a joint.

The phrase “osteoarthritic pain” means joint pain in an osteoarthritic joint.

The phrase “rheumatoid arthritic pain” means joint pain in a rheumatoid arthritic joint.

The phrase “inflammatory pain” means pain in a tissue that also exhibits edema or swelling, including inflammatory joint pain. Inflammatory joint pain includes rheumatoid arthritic joint pain.

The phrase “acute pain” means any pain, including, but not limited to, joint pain, osteoarthritic pain, rheumatoid arthritic pain, inflammatory pain, pain from a burn, pain from a cut, surgical pain, pain from fibromyalgia, bone cancer pain, menstrual pain, back pain, headache, static allodynia, and dynamic allodynia, that lasts from 1 minute to 91 days, 1 minute to 31 days, 1 minute to 7 days, 1 minute to 5 days, 1 minute to 3 days, 1 minute to 2 days, 1 hour to 91 days, 1 hour to 31 days, 1 hour to 7 days, 1 hour to 5 days, 1 hour to 3 days, 1 hour to 2 days, 1 hour to 24 hours, 1 hour to 12 hours, or 1 hour to 6 hours, per occurrence if left untreated. Acute pain includes, but is not limited to, joint pain, osteoarthritic pain, rheumatoid arthritic pain, inflammatory pain, pain from a burn, pain from a cut, surgical pain, pain from fibromyalgia, bone cancer pain, menstrual pain, back pain, headache, static allodynia, dynamic allodynia, acute joint pain, acute osteoarthritic pain, acute rheumatoid arthritic pain, acute inflammatory pain, acute headache, acute menstrual pain, acute back pain, and acute pain from fibromyalgia. Acute pain may be selected from acute joint pain, acute osteoarthritic pain, acute rheumatoid arthritic pain, acute inflammatory pain, acute headache, acute menstrual pain, and acute back pain. Acute pain may be selected from acute joint pain, acute osteoarthritic pain, acute rheumatoid arthritic pain, and acute inflammatory pain. Acute pain may be selected from acute joint pain, acute osteoarthritic pain, and acute rheumatoid arthritic pain. Acute pain may be selected from acute joint pain and acute osteoarthritic pain.

It should be appreciated that alleviating acute pain means having an appreciable pain alleviating effect within 91, 31, 7, 5, 3, or 2 days, or 24, 12, 6, 3, 2, 1, 0.5, 0.25, 0.20. 0.17, or 0.10 hours after administering the first dose of an active ingredient.

The phrase “chronic pain” means any pain, including, but not limited to, joint pain, osteoarthritic pain, rheumatoid arthritic pain, inflammatory pain, pain from a burn, pain from a cut, surgical pain, pain from fibromyalgia, bone cancer pain, menstrual pain, back pain, headache, static allodynia, dynamic allodynia, that lasts longer than 91 days, 6 months, 1 year, 5 years, or 10 years per occurrence if left untreated. Chronic pain may be selected from chronic joint pain, chronic osteoarthritic pain, chronic rheumatoid arthritic pain, chronic inflammatory pain, chronic headache, chronic menstrual pain, chronic back pain, and chronic pain from fibromyalgia. Chronic pain may be selected from chronic joint pain, chronic osteoarthritic pain, chronic rheumatoid arthritic pain, chronic inflammatory pain, chronic headache, chronic menstrual pain, and chronic back pain. Chronic pain may be selected from chronic joint pain, chronic osteoarthritic pain, chronic rheumatoid arthritic pain, and chronic inflammatory pain. Chronic pain may be selected from chronic joint pain, chronic osteoarthritic pain, and chronic rheumatoid arthritic pain. Chronic pain may be selected from chronic joint pain and chronic osteoarthritic pain.

It should be appreciated that alleviating chronic pain means having an appreciable pain alleviating effect within 91, 60, 31, 28, 21, 14, 7, 3, or 2 days or 24, 12, 6, 3, 2, 1, 0.5, 0.25, 0.20. 0.17, or 0.10 hours after administering the first dose of active ingredient.

In a clinical setting, a physician may assess a patients need for, or response to, treatment of osteoarthritis, rheumatoid arthritis, impaired joint function, pain, including osteoarthritic pain, rheumatoid arthritic pain, acute pain, joint pain, chronic pain, inflammatory pain, pain by administering a standard assessment questionnaire such as WOMAC or the Patient Global Impression of Change (“PGIC”).

Further with respect to the assessment of a patients need for, or response to, treatment of the aforementioned pain states and abdominal aortic aneurysm pain, skin ulcer pain, or cancer pain, the physician may apply a pain assessment scale such as the Visual Analog Scale (“VAS”), wherein a patient is asked to indicate a point on a 100 millimeter line, having a left anchor of no pain and a right anchor of worst possible pain, corresponding to their degree of pain or the Likert score, wherein a patient is asked to categorize their pain on a numerical scale of from 0 (no pain) to 10 (worst possible pain).

The phrases “therapeutically effective amount” and “effective amount” are synonymous and mean an amount of an active compound or combination or pharmaceutical composition of this invention as described above sufficient to alleviate, eliminate, inhibit or prevent the onset, or inhibit the progress, prevent further progress, or reverse progression, in part or in whole, of any one or more pathological hallmarks or symptoms of the disease or disorder that is appreciated or suspected or expected in the particular patient being treated or prevented.

It should be appreciated that a therapeutically effective or effective amount means an amount sufficient to have a desired effect in a patient to which that amount has been administered. For illustrative example, where cartilage damage is being inhibited, a therapeutically effective amount includes a cartilage damage inhibiting effective amount. Where osteoarthritis is being treated, a therapeutically effective amount includes an osteoarthritis treating effective amount. Where pain is being alleviated, a therapeutically effective amount includes a pain alleviating effective amount. Where osteoarthritic or rheumatoid arthritic pain is being alleviated, a therapeutically effective amount includes an osteoarthritic or rheumatoid arthritic pain alleviating effective amount, respectively.

A therapeutically effective amount, or, simply, effective amount, of an invention combination will generally be from about 1 to about 300 mg/kg of subject body weight of each of the allosteric inhibitor of MMP-13, or a pharmaceutically acceptable salt thereof, and the alpha-2-delta receptor ligand. Typical doses will be from about 10 to about 5000 mg/day for an adult subject of normal weight for each component of the combination. In a clinical setting, regulatory agencies such as, for example, the Food and Drug Administration (“FDA”) in the U.S. may require a particular therapeutically effective amount.

A therapeutic pain alleviating effect is the effect of one or more of the compounds or combinations according to the invention method as defined above that eliminates, or inhibits or prevents onset of, suppresses, reduces, prevents, or otherwise inhibits, pain in a patient, including, but not limited to, the suppression, reduction, prevention, inhibition or elimination of pain symptoms due to, for example, cartilage damage, acute pain, chronic pain, mechanical pain, static allodynia, dynamic allodynia, bone cancer pain, headache, osteoarthritic pain, rheumatoid arthritic pain, inflammatory pain, or pain associated with autoimmune disorders or fibromyalgia.

In determining what constitutes an effective amount or a therapeutically effective amount of an invention combination for treating, preventing, or reversing one or more symptoms of any one of the diseases and disorders described above that are being treated according to the invention methods, a number of factors will generally be considered by the medical practitioner or veterinarian in view of the experience of the medical practitioner or veterinarian, including the Food and Drug Administration guidelines, or guidelines from an equivalent agency, published clinical studies, the subject's (e.g., mammal's) age, sex, weight and general condition, as well as the type and extent of the disease, disorder or condition being treated, and the use of other medications, if any, by the subject. As such, the administered dose may fall within the ranges or concentrations recited above, or may vary outside them, ie, either below or above those ranges, depending upon the requirements of the individual subject, the severity of the condition being treated, and the particular therapeutic formulation being employed. Determination of a proper dose for a particular situation is within the skill of the medical or veterinary arts. Generally, treatment may be initiated using smaller dosages of the invention combination that are less than optimum for a particular subject. Thereafter, the dosage can be increased by small increments until the optimum effect under the circumstance is reached. For convenience, the total daily dosage may be divided and administered in portions during the day, if desired.

The terms “mammal” and “patient” are synonymous and include humans, companion animals such as cats and dogs, primates such as monkeys and chimpanzees, and livestock animals such as horses, cows, pigs, and sheep. In one aspect of this invention, the mammal is a human. In another aspect, the mammal is a cat or dog. In still another aspect, the mammal is a cow, horse, pig, or sheep. In still another aspect, the mammal is a laboratory animal such as a rat, mouse, rabbit, guinea pig, dog, cat, monkey, hamster, or transgenic variants thereof.

The phrase “livestock animals” as used herein refers to domesticated quadrupeds, which includes those being raised for meat and various byproducts, e.g., a bovine animal including cattle and other members of the genus Bos, a porcine animal including domestic swine and other members of the genus Sus, an ovine animal including sheep and other members of the genus Ovis, domestic goats and other members of the genus Capra; domesticated quadrupeds being raised for specialized tasks such as use as a beast of burden, e.g., an equine animal including domestic horses and other members of the family Equidae, genus Equus, or for searching and sentinel duty, e.g., a canine animal including domestic dogs and other members of the genus Canis; and domesticated quadrupeds being raised primarily for recreational purposes, e.g., members of Equus and Canis, as well as a feline animal including domestic cats and other members of the family Felidae, genus Felis.

The term “drug”, which is synonymous with the phrases “active component,” “active compound,” “active ingredient,” “therapeutically active agent,” and the like, means an allosteric inhibitor of MMP-13 named above, or a pharmaceutically acceptable salt thereof, or an alpha-2-delta receptor ligand, or a pharmaceutically acceptable salt thereof, and may further mean another therapeutic agent described above.

It should be appreciated that pregabalin, also known as (S)-3-(aminomethyl)-5-methylhexanoic acid, has the structure drawn below: 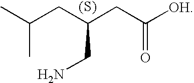
Pregabalin has been in late-stage clinical trials for the adjunctive treatment of epilepsy and the treatment of neuropathic pain and generalized anxiety disorder.

It should be appreciated that the compound named (3S,4S)-(1-Aminomethyl-3,4-dimethyl-cyclopentyl)-acetic acid is also known by the names (S,S)-(1-aminomethyl-3,4-dimethyl-cyclopentyl)-acetic acid and (3S,4S)-1-(aminomethyl)-cyclopentaneacetic acid. The compound named (3S,4S)-(1-Aminomethyl-3,4-dimethyl-cyclopentyl)-acetic acid has the structure drawn immediately below:

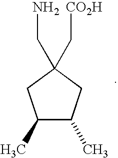

It should be appreciated that the alpha-2-delta receptor ligand named 3-(1-aminomethyl-cyclohexylmethyl)-4H-[1,2,4]oxadiazol-5-one hydrochloride is also known as “CI-1045”.

Gabapentin has previously been approved by the United States Food and Drug Administration (“FDA”) and is currently marketed in the U.S. under the tradename NEURONTIN® for the adjunctive treatment of epilepsy and post-herpetic pain.

The term “NSAID” is an acronym for the phrase “nonsteroidal anti-inflammatory drug”, which means any compound that inhibits cyclooxygenase-1 (“COX-1”) and cyclooxygenase-2. Most NSAIDs fall within one of the following five structural classes: (1) propionic acid derivatives, such as ibuprofen, naproxen, naprosyn, diclofenac, and ketoprofen; (2) acetic acid derivatives, such as tolmetin and sulindac; (3) fenamic acid derivatives, such as mefenamic acid and meclofenamic acid; (4) biphenylcarboxylic acid derivatives, such as diflunisal and flufenisal; and (5) oxicams, such as piroxim, peroxicam, sudoxicam, and isoxicam. Other useful NSAIDs include aspirin, acetominophen, indomethacin, and phenylbutazone. Selective inhibitors of cyclooxygenase-2 such as celecoxib (e.g., CELEBREX® by G. D. Searle & Co., Skokie Ill.), valdecoxib (e.g., BEXTRA® by Pharmacia & Upjohn Company, Peapack, N.J.), rofecoxib, (e.g., VIOXX® by MERCK & CO., Inc., Whitehouse Station, N.J.), lumiracoxib (e.g., PREXIGE® by Novartis AG, Basel, Switzerland), parecoxib (DYNASTAT® by G. D. Searle & Co., Skokie Ill.), etoricoxib (ARCOXIA® by MERCK & CO., Inc., Whitehouse Station, N.J.), carprofen (RIMADYL® by Pfizer, Inc., New York, N.Y.), deracoxib (DERAMAXX® by Novartis AG, Basel, Switzerland), and the like are also considered to be NSAIDs.

The term “Thr245” means threonine 245 of an MMP-13 enzyme.

The term “Thr247” means threonine 247 of an MMP-13 enzyme.

The term “Met253” means methionine 253 of an MMP-13 enzyme.

The term “His251” means histidine 251 of an MMP-13 enzyme.

It should be appreciated that the matrix metalloproteinases include, but are not limited to, the following enzymes:

MMP-1, also known as interstitial collagenase, collagenase-1, or fibroblast-type collagenase;

MMP-2, also known as gelatinase A or 72 kDa Type IV collagenase;

MMP-3, also known as stromelysin or stromelysin-1;

MMP-7, also known as matrilysin or PUMP-1;

MMP-8, also known as collagenase-2, neutrophil collagenase or polymorphonuclear-type (“PMN-type”) collagenase;

MMP-9, also known as gelatinase B or 92 kDa Type IV collagenase;

MMP-10, also known as stromelysin-2;

MMP-11, also known as stromelysin-3;

MMP-12, also known as metalloelastase;

MMP-13, also known as collagenase-3;

MMP-14, also known as membrane-type (“MT”) 1-MMP or MT1-MMP;

MMP-15, also known as MT2-MMP;

MMP-16, also known as MT3-MMP;

MMP-17, also known as MT4-MMP;

MMP-18; and

MMP-19.

Other known MMPs include MMP-26 (Matrilysin-2).

It should be appreciated that selectivity of an allosteric inhibitor of MMP-13, or a pharmaceutically acceptable salt thereof, is a multifactorial characteristic that includes the number of other MMP enzymes and TACE over which selectivity for MMP-13 inhibition is present and the degree of selectivity of inhibition of MMP-13 over another particular MMP or TACE, as measured by, for example, the IC₅₀ in micromolar concentration of inhibitor for the inhibition of the other MMP enzyme or TACE divided by the IC₅₀ in micromolar concentration of inhibitor for the inhibition of MMP-13.

It should be appreciated that the S1′ site of MMP-13 was previously thought to be a grossly linear channel which contained an opening at the top that allowed an amino acid side chain from a substrate molecule to enter during binding, and was closed at the bottom. Applicant points out that the S1′ site is actually composed of an S1′ channel angularly connected to a newly discovered pocket which applicant calls the S1″ site. The S1″ site is open to solvent at the bottom, which can expose a functional group of the instant allosteric MMP-13 inhibitors to solvent. For illustrative purposes, the S1′ site of the MMP-13 enzyme can now be thought of as being like a sock with a hole in the toe portion, wherein the S1′ channel is the region from approximately the opening to the ankle region, and the S1″ site is the foot region below the ankle region, which foot region is angularly connected to the ankle region.

More particularly, the S1′ channel is a specific part of the S1′ site and is formed largely by Leu218, Val219, His222 and by residues from Leu239 to Tyr244. The S1″ binding site is defined by residues from Tyr246 to Pro255. The S1″ site contains at least two hydrogen bond donors and aromatic groups which interact with a compound which is an allosteric inhibitor of MMP-13.

Without wishing to be bound by any particular theory, the inventor believes that the S1″ site could be a recognition site for triple helix collagen, the natural substrate for MMP-13. It is possible that the conformation of the S1″ site is modified only when an appropriate compound binds to MMP-13, thereby interfering with the collagen recognition process. This pattern of binding offers the possibility of greater selectivity than what is achievable with the binding pattern of non-allosteric, selective inhibitors of MMP-13, wherein the non-allosteric, selective binding pattern requires ligation of the catalytic zinc atom at the active site and occupation the S1′ channel, but not the S1″ site.

All that is required to practice the method of this invention is to administer an invention pharmaceutical composition, in an amount that is therapeutically effective for preventing, inhibiting, or reversing the condition(s) being treated. The invention combination can be administered directly or in a pharmaceutical composition as described below.

Pharmaceutical Compositions of the Invention:

Pharmaceutical compositions, described briefly here and more fully below, of an invention combination may be produced by formulating the invention combination in dosage unit form with a pharmaceutical carrier. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses. Alternatively, the active components of the invention combination may be formulated separately.

Some examples of suitable pharmaceutical carriers, including pharmaceutical diluents, are gelatin capsules; sugars such as lactose and sucrose; starches such as corn starch and potato starch; cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations.

The pharmaceutical compositions to be employed in the invention can also contain other components such as coloring agents, flavoring agents, and/or preservatives. These materials, if present, are usually used in relatively small amounts. The pharmaceutical compositions can, if desired, also contain other therapeutic agents commonly employed to treat any of the above-listed diseases and disorders.

The percentage of the active ingredients in the foregoing pharmaceutical compositions can be varied within wide limits, but for practical purposes they usually are present in a total concentration of at least 10% in a solid composition and at least 2% in a primary liquid composition. The most satisfactory pharmaceutical compositions are those in which a much higher proportion of the active ingredients are present, for example, in a total concentration of up to about 98%.

In one aspect, routes of administration of a combination of this invention are oral or parenteral. However, other routes of administration may be utilized depending upon the condition being treated. For example, topical administration or administration by injection may be utilized for treating conditions localized to the skin or a joint. Administration by transdermal patch may be utilized where, for example, it is desirable to effect sustained dosing. Where active components of the invention combination are formulated separately, different routes of administration for each component may be used.

It should be appreciated that the different routes of administration may require different dosages. For example, a useful intravenous (“IV”) dose is between 5 and 50 mg, and a useful oral dosage is between 20 and 800 mg, both for each of the active ingredients. The dosage is within the dosing range used in treatment of the above-listed diseases, or as would be determined by the needs of the patient as described by the physician.

The invention combination may be administered in any form. In one aspect of this invention, administration is in unit dosage form. A unit dosage form of the invention combination to be used in this invention may also comprise other compounds useful in the therapy of diseases described above. A further description of pharmaceutical formulations useful for administering the invention combinations is provided below.

The active components of the invention combination and other compounds as described above, if any, may be formulated together or separately and may be administered together or separately. The particular formulation and administration regimens used may be tailored to the particular patient and condition being treated by a practitioner of ordinary skill in the medical or pharmaceutical arts.

It should be appreciated that a drug that has been previously administered as monotherapy or as adjunctive therapy at a particular dose or number of times per day to a mammal may be administered to the mammal as part of a combination or pharmaceutical composition according to this invention at the same or a different dose and the same or a different number of times per day.

Certain Advantages of the Invention:

The advantages of using an invention combination in a method of the instant invention include the nontoxic nature of the active compounds which comprise the combination at and substantially above therapeutically effective doses, their ease of preparation, the fact that the compounds are well-tolerated, and the ease of topical, IV, or oral administration of the drugs.

Another important advantage is that the present invention combinations more effectively target a particular disease that is responsive to both treatment with an inhibitor of MMP-13 and an alpha-2-delta receptor ligand than would a single component of the combination alone.

Further, this “one-two” punch will exhibit fewer undesirable side effects than combinations that contain non-selective inhibitors of MMP-13, which have side effects due to a broader spectrum of MMP enzyme biological activities. For example, virtually all prior art MMP inhibitors tested clinically to date have exhibited an undesirable side effect known as muscoloskeletal syndrome (“MSS”). MSS is associated with administering an inhibitor of multiple MMP enzymes or an inhibitor of a particular MMP enzyme such as MMP-1. MSS will be significantly reduced in type and severity by administering the invention combination.

This advantage of the instant combinations will also significantly increase the likelihood that agencies which regulate new drug approvals, such as the United States Food and Drug Administration, will approve the instant combination versus the competing combination discussed above even in the unlikely event that the two combinations behaved similarly in clinical trials. These regulatory agencies are increasingly aware that clinical trials, which test drug in limited population groups, do not always uncover safety problems with a drug, and thus all other things being equal, the agencies will favor the drug with the lowest odds of producing undesirable side effects.

Another important advantage is that the independent anti-inflammatory and pain reducing properties of alpha-2-delta receptor ligands, the cartilage modifying properties of alpha-2-delta receptor ligands, and the cartilage and extracellular matrix modifying properties of allosteric inhibitors of MMP-13 provide patients suffering from diseases or disorders such as cartilage damage, osteoarthritis, joint pain, rheumatoid arthritis, inflammation, breast cancer metastasis and pain, and bone cancer metastasis and pain with both relief of symptoms and prevention or inhibition of the underlying disease pathology such as cartilage degradation in OA or extracellular matrix degradation/penetration in breast cancer metastasis.

A further advantage of the invention combination is administration of the invention combination to treat a disease or disorder in a mammal may allow lower doses of active ingredients of the combination to be used than would be used if each ingredient were administered alone, thereby reducing undesirable side effects, if any.

Another important advantage is that the combinations of this invention are useful in the prevention (by prophylactic administration) and treatment of human medical and animal veterinary diseases and disorders.

Preparation of Active Ingredients:

Preparation of any allosteric inhibitor of MMP-13 or ligand to an alpha-2-delta receptor named above is readily carried out, either commercially, or by synthetic methodology well known to those skilled in the art of organic chemistry. Preparations are also found in the above-referenced patent documents.

Certain allosteric inhibitors of MMP-13 may also be prepared as described below in Schemes 1 to 4.

Biaryl, aryl-heteroaryl, and the like allosteric inhibitors of MMP-13 may also be prepared as described below in Scheme 1. 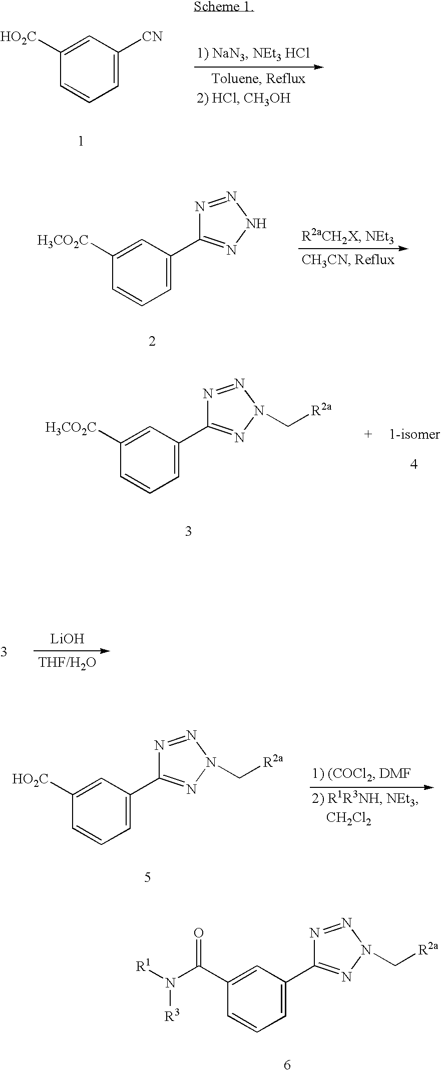
wherein R¹, R^{2a}, and R³ are as defined above for G₁ and G₂.

The procedure of Scheme 1 is exemplified below in Preparations 1 and 2.

### PREPARATION 1

4-({3-[2-(4-Methoxy-benzyl)-2H-tetrazol-5-yl]-benzoyl amino}-methyl)-benzoic acid 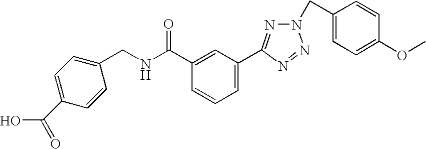
Step (a): 3-(2H-Tetrazol-5-yl)benzoic acid methyl ester

To a solution of 3-cyanobenzoic acid (12.3 g, 0.083 mol) in toluene (300 mL) were added sodium azide (16 g, 0.25 mol) and triethylamine hydrochloride (34 g, 0.25 mol) respectively. The reaction mixture was refluxed for 4 hours, cooled to room temperature, and diluted with water (300 mL). The organic phase was separated and the aqueous portion was acidified (pH=1) using concentrated HCl. The precipitate was collected by filtration and oven-dried to give 14 g (89%) of the tetrazole as a white solid. CI-MS: C8H6N402 [M+1] 191.0. The product obtained (14 g, 0.074 mol) was suspended in anhydrous methanol followed by the addition of gaseous HCl over a period of 20 minutes. The warm solution was stirred at room temperature for overnight, then concentrated in vacuo. The resulting residue was triturated with diethyl ether and collected by filtration to yield 12.1 g (81%) of the methyl ester intermediate 2. CI-MS: C9H8N402 [M+1] 205.2

Step (b): 3-[2-(4-Methoxy-benzyl)-2H-tetrazol-5-yl]-benzoic acid methyl ester

The methyl ester synthesized in Step (a) (12.1 g, 0.059 mol) was diluted with acetonitrile (300 niL) and triethylamine (6.6 g, 0.060 mol). Dissolution occurred after stirring at room temperature for 5 minutes. The solution was treated with 4-methoxybenzyl chloride (6.6 g, 0.065 mol) and refluxed for overnight. Precipitation occurred on cooling the reaction mixture to room temperature. The solvent was concentrated and the residue was triturated with ethyl acetate and filtered. The filtrate was washed with aqueous HCl (1M, 50 mL), dried (MgSO4), and concentrated in vacuo. The 2-isomer was isolated analytically pure utilizing silica gel chromatography (elution with dichloromethane) to give the title compound (10.5 g, 55%) as a white solid. ¹HNMR (CDCl3) δ8.8 (s, 1H), 8.3 (d, 1H), 8.1 (d, 1H), 7.5 (t, 1H), 7.2 (d, 2H), 6.9 (d, 2H), 5.7 (s, 2H), 3.9 (s, 3H), 3.8 (s, 3H) ppm. Mp 105-106° C.

Step (c): 3-[2-(4-Methoxy-benzyl)-2H-tetrazol-5-yl]-benzoic acid

The ester prepared in Step (b) (10.4 g, 0.032 mol) was suspended in aqueous tetrahydrofuran (20 mL, 1:1) followed by the addition of lithium hydroxide monohydrate (4 g, 0.096 mol) in one portion. Dissolution occurred after stirring at room temperature for 30 minute. The solution was stirred for an additional 16 hours. The THF was concentrated in vacuo and the aqueous solution was acidified to pH=1 using concentrated HCl. The resulting precipitate was collected by filtration and recrystallized from hexane/ethyl acetate to give the title 2compound (10 g, 100%) as a white solid. ¹HNMR (DMSO-d6) δ8.6 (s, 1H), 8.3 (d, 1H), 8.1 (d, 1H), 7.7 (t, 1H), 7.4 (d, 2H), 6.9 (d, 2H), 6.9 (s, 2H), 3.7 (s, 3H) ppm.

Step (d): 3-[2-(4-Methoxy-benzyl)-2H-tetrazol-5-yl]-benzoyl chloride.

The carboxylic acid intermediate (10 g, 0.032 mol) from Step (c) was suspended in dichloromethane followed by the addition of oxalyl chloride (20.4 g, 0.16 mol) and catalytic DMF. The reaction mixture was stirred at room temperature for 3 hours, at which time dissolution was nearly complete. The reaction mixture was filtered and concentrated in vacuo. The residue was triturated with petroleum ether and collected by filtration to give the title compound (9.5 g, 90%) as a white solid. ¹HNMR (CDCl3) δ8.9 (s, 1H), 8.5 (d, 1H), 8.2 (d, 1H), 8.6 (t, 1H), 7.4 (d, 2H), 6.9 (d, 2H), 3.8 (s, 3H) ppm. Mp 122-124° C.

Step (e): 4-({3-[2-(4-Methoxy-benzyl)-2H-tetrazol-5-yl]-benzoylamino}-methyl)-benzoic acid methyl ester

To a solution of methyl 4-(aminomethyl)benzoate hydrochloride (0.22 g, 1.1 mmol) and triethylamine (0.22 g, 2.2 mmol) in dichloromethane (20 mL) was added the acid chloride (0.33 g, 1.01 mmol) prepared in Step (d). The reaction mixture was stirred at room temperature for 16 hour, then diluted with aqueous HCl (1M, 20 mL). The organic phase was separated, washed with brine, dried (MgSO4), and concentrated in vacuo. The residue was recrystallized from hexane/ethyl acetate to give a white solid (0.38 g, 83%). ¹HNMR (DMSO-d6) δ9.4 (t, 1H), 8.5 (s, 1H), 8.2 (d, 1H), 8.1 (d, 1H), 7.9 (d, 2H), 7.8 (t, 1H),7.5 (d, 2H), 7.4 (d, 2H), 6.9 (d, 2H), 5.9 (s, 2H), 4.5 (d, 2H), 3.8 (s, 3H), 3.7 (s, 3H) ppm. Mp 167-168° C.

Step (f): 4-({3-[2-(4-Methoxy-benzyl)-2H-tetrazol-5-yl]-benzoylamino}-methyl)-benzoic acid

The ester (0.065 g, 0.14 mmol) prepared in Step (e) was diluted with aqueous tetrahydrofuran followed by the addition of lithium hydroxide monohydrate (0.018 g, 0.4 mmol). Following the experimental conditions described in (d) yielded the free acid (0.045 g, 71%)as a white solid. ¹HNMR (DMSO-d6) δ12.8 (bs, 1H), 9.4 (t, 1H), 8.5 (s, 1H), 8.2 (d, 1H), 8.0 (d, 1H), 7.9 (d, 2H), 7.7 (t, 1H), 7.4 (dd, 4H), 6.9 (d, 2H), 5.9 (s, 2H), 4.5 (d, 2H), 3.7 s, 3H) ppm. Mp 202-205° C.

### PREPARATION 2

4-{5-[3-(3-Phenyl-prop-1-ynyl)-phenyl]-tetrazol-2-ylmethyl}-benzoic acid

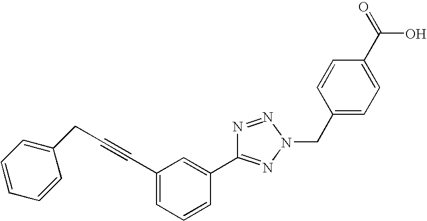

Step (a): 5-(3-Iodo-phenyl)-2H-tetrazole.

The 3-iodobenzonitrile (3.6 g, 16.6 mmol) was converted to the corresponding tetrazole (4.1 g, 91%) utilizing reaction conditions previously described in Step (a) of Preparation 1. CI-MS: C₇H₅IN₂ [M+1] 273.0.

Step (b): 4-[5-(3-Iodo-phenyl)-tetrazol-2-ylmethyl]-benzoic acid tert-butyl ester.

The tetrazole (4 g, 14.7 mmol) prepared in Step (a) was alkylated using the reaction conditions previously described in Step (b) of Preparation 1 to give analytically pure 2-regioisomer (3 g, 44%) and the 1-regioisomer (0.54 g, 8%) respectively. ¹HNMR (CDCl3) 2-regioisomer δ8.5 (s, 1H), 8.1 (d, 1H), 8.0 (d, 2H), 7.8 (d, 1H), 7.4 (d, 2H), 7.2 (m, 2H), 5.8 (s, 2H), 1.6 (s, 9H) ppm. ¹HNMR (CDCl3) 1-regioisomer δ8.0 (d, 2H), 7.9 (d, 2H), 7.5 (d, 1H), 7.3-7.1 (m, 3H), 5.6 (s, 2H) 1.6 (s, 9H) ppm.

Step (c): 4-[5-(3-Iodo-phenyl)-tetrazol-2-ylmethyl]-benzoic acid.

The ester (2.5 g, 5.41 mmol) prepared in Step (b) was suspended in dichloromethane (20 mL) followed by the addition of trifluoroacetic acid (5 mL). The solution was stirred for 16 hours at 25° C., then concentrated in vacuo. The resulting white solid was triturated with hexane/diethyl ether and the carboxylic acid (2.1 g, 100%) was collected by filtration. Mp 241-242° C.

Step (d): 4-{5-[3-(3-Phenyl-prop-1-ynyl)-phenyl]-tetrazol-2-ylmethyl}-benzoic acid. Mp 195-198° C.

The iodo derivative (1 g, 2.46 mmol) prepared in Step (c) was dissolved in dimethylformamide (10 mL) followed by the addition of diisopropylethylamine (1.3 g, 9.8 mmol), copper (I) iodide (0.17 g, 0.89 mmol), 3-phenyl-1-propyne (0.40 g, 3.4 mmol), and bis(triphenylphosphine) palladium (II) dichloride (0.34 g, 0.49 mmol). The reaction mixture was stirred at 50° C. for 4 hours under an atmosphere of N₂. The dark reaction mixture was cooled to room temperature and diluted with equal volumes of ethyl acetate and aqueous HCl. The organic phase was separated, washed with brine, dried (MgSO4) and concentrated in vacuo. The liquid obtained was purified using silica gel chromatography (elution with dichloromethane/tetrahydrofuran) to give a cream colored solid (0.37 g, 38%). ¹HNMR (DMSO-d6) δ13.0 (bs, 1H), 8.0-7.9 (m, 4H), 7.7-7.2 (m, 9H), 6.1 (s, 2H), 3.9 (s, 2H) ppm.

Isoquinoline derivatives may be prepared as outlined below in Scheme 2. 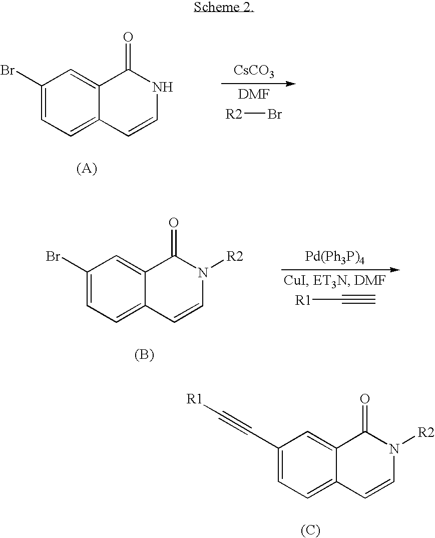
wherein R¹ and R² are as defined above for G₁ and G₂.

In Scheme 2, a suspension of a 7-bromo-1-hydroxyisoquinoline (A) can be alkylated in an aprotic solvent such as dimethylformamide when treated with a common alkylating agent such as an alkyl halide or benzyl halide, generally in the presence of a base such as cesium carbonate, potassium carbonate, or triethylamine. The alkylated isoquinoline (B) can be further reacted with a variety of alkynes using standard coupling conditions known to those skilled in the art, for example, using a catalyst such as Pd(PPh₃)₄ or PdCl₂((PPh₃)₂, with or without an accompanying ligand, and in the presence of a base, such as triethylamine or diisopropylamine, to give compounds of this invention (C). Where appropriate, cleavage of t-butyl protecting groups is carried out under standard conditions, for example, moderately acidic hydrolysis, to afford the carboxylic acid.

The method illustrated in Scheme 2 is exemplified below in Preparation 3.

### PREPARATION 3

4-[1-Oxo-7-(3-phenyl-prop-1-ynyl)-1H-isoquinolin-2-ylmethyl]benzoic acid

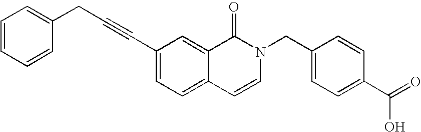

Step (1): 4-(7-bromo-1-oxo-1H-isoquinolin-2-ylmethyl)benzoic acid tert-butyl ester

A suspension of 7-bromo-1-hydroxyisoquinoline (7.02 g, 28.2 mmol) in dimethylformamide (75 mL) was treated with 4-bromomethylbenzoic acid tert-butyl ester (12.5 g, 36.7 mmol) and cesium carbonate (11.94 g, 36.7 mmol), then stirred overnight at room temperature. The dimethylformamide was evaporated in vacuo, the residue diluted with ethyl acetate, washed with 1N HCl, the organic portion washed with brine, dried over MgSO₄ and evaporated to dryness. The residue was triturated with hot hexanes/ethyl acetate, cooled to room temperature, and the solid collected by filtration and dried to give 8.77 g of the product as white crystals (75.7% yield).

¹H-NMR (CDCl₃); d 8.58 (d, 1H), 7.95-7.93 (d, 2H), 7.73-7.71 (dd, 1H), 7.39-7.32 (m, 3H), 7.07-7.05 (d, 1H), 6.46-6.44 (d, 1H), 5.24 (s, 2H), 1.57 (s, 9H). MS: M⁺+1=414.0/416.0 Da

Step (2): 4-[1 -oxo-7-(3-phenyl-prop-1-ynyl)-1H-isoquinolin-2-ylmethyl]benzoic acid tert-butyl ester

A solution of 4-(7-bromo-1-oxo-1H-isoquinolin-2-ylmethyl)benzoic acid tert-butyl ester (1.50 g, 3.62 mmol) in dimethylformamide (10 mL) was degassed with nitrogen, then treated with triethyl-amine (2.07 mL, 14.8 mmol), CuI (0.050 g, 0.26 mmol), Pd(Ph₃P)₄ (0.173 g, 0.15 mmol), and 3-phenyl-1-propyne (1.13 mL, 9.05 mmol). The reaction mixture was heated in an oil bath at 65° C. for 5 hours, cooled to room temperature, the DMF was evaporated, and the residue dissolved in ethyl acetate. The solution was washed with 1N HCl , brine, dried over MgSO₄ and evaporated onto silica gel. Purification on a 3.5×18 cm silica gel column eluted with hexane/ethyl acetate 4:1, followed by drying, afforded the product as 1.0 g of bright yellow solid (67.6% yield).

¹H-NMR (CDCl₃); d 8.53 (d, 1H), 7.95-7.93 (d, 2H), 7.67-7.65 (dd, 1H), 7.43-7.41 (d, 3H), 7.36-7.31 (m, 4H), 7.28-7.24 (m, 1H), 7.04-7.03 (d, 1H), 6.46-6.44 (d, 1H), 5.24 (s, 2H), 3.86 (s, 2H), 1.57 (s, 9H). MS: M⁺+1=450.2 Da

Step (3): 4-[1-Oxo-7-(3-phenyl-prop-1-ynyl)-1H-isoquinolin-2-ylmethyl]benzoic acid

A solution of 4-[1-oxo-7-(3-phenyl-prop-1-ynyl)-1H-isoquinolin-2-ylmethyl]benzoic acid tert-butyl ester [0.20 g, 0.44 mmol, Step (2)] was treated with trifluoroacetic acid (5 mL) and the reaction mixture stirred at room temperature for 30 minutes. The solution was evaporated to dryness, the residue dissolved in ethyl acetate, washed with water, brine, dried over MgSO₄ and evaporated to dryness. The brown solid was triturated with acetonitrile, the solid collected by filtration, and washed with acetonitrile. The solid was then dissolved in hot ethyl acetate, evaporated onto silica gel, and purified on a 2.5×10 cm silica gel column eluted with hexanes/ethyl acetate 1:1, followed by ethyl acetate. Drying afforded the purified product as 0.020 g of light yellow solid (11.4% yield). ¹H-NMR (DMSO-d₆); d 8.20 (s, 1H), 7.89-7.87 (d, 2H), 7.73-7.60 (m, 3H),7.42-7.33 (m, 6H), 7.26-7.22 (m, 1H), 6.69-6.67 (d, 1H), 5.23 (s, 2H), 3.92 (s, 2H). MS: M⁺+1=394.1 Da

Pyrimidine-2,4-dione allosteric inhibitors of MMP-13 may be prepared as illustrated below in Scheme 3. 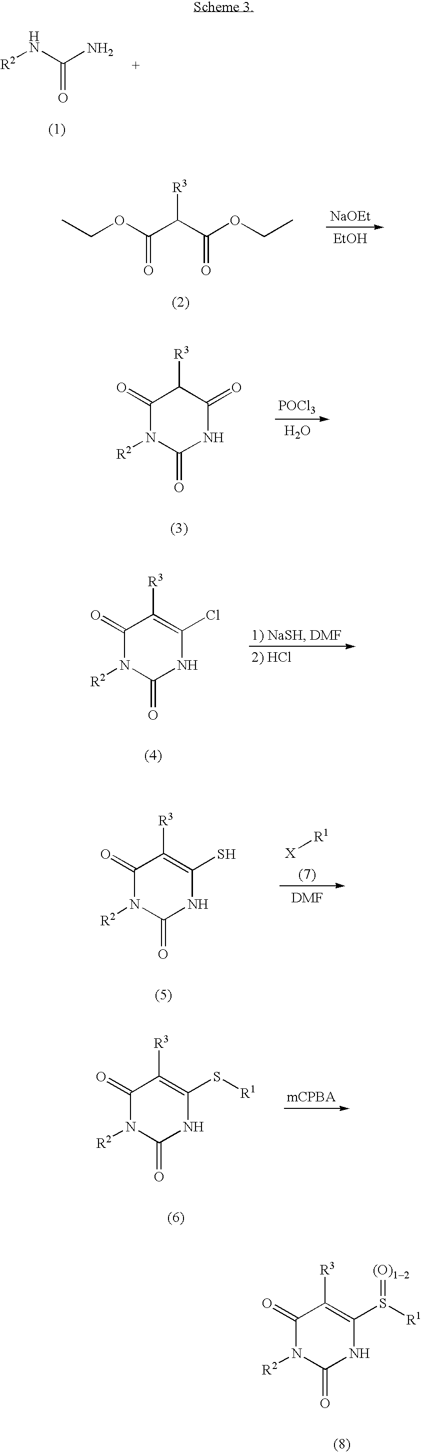
wherein R₁ and R² are as defined above for G₁ and G₂ and R³ is methyl, fluoro, and the like.

The method illustrated in Scheme 3 is exemplified below in Preparation 4.

### PREPARATION 4

4-[4-(Biphenyl-3-ylmethylsulfanyl)-5-methyl-2,6-dioxo-3,6-dihydro-2H-pyrimidin-1-ylmethyl]-benzoic acid

Step (A): 4-(4-Chloro-5-methyl-2,6-dioxo-3,6-dihydro-2H-pyrimidin-1-ylmethyl)- benzoic acid tert-butyl ester

5-Methyl-6-chloropyrimidine-2,4-dione (1.00 g, 6.22 mmol) was dissolved in dimethylformamide (15 mL).To the solution was added cesium carbonate (2.02 g, 6.22 mmol) and t-butyl 4-bromomethylbenzoate (1.69 g, 6.22 mmol). The mixture was stirred at room temperature for 20 hours. The solvent was evaporated under a vacuum at 60° C. The residue was mixed with tetrahydrofuran (50 mL) and methanol (10 mL) and filtered. The filtrate was evaporated in a vacuum to an amber oil. The oil was purified by flash chromatography on silica gel eluting first with dichloromethane then with dichloromethane:methanol (19:1) to give the desired product (0.88 g, 40% yield). ¹H-NMR (DMSO-d₆) δ11.8 (s, 1H), 7.85 (d, 2H), 7.34 (d, 2H), 5.22 (s,2H), 1.88 (s, 3H), 1.51 (s, 9H).

Step (B): 4-(4-Mercapto-5-methyl-2,6-dioxo-3,6-dihydro-2H-pyrimidin-1-ylmethyl)- benzoic acid tert-butyl ester

The product of Step (A) (0.54 g) was dissolved in dimethylformamide (5 mL) wand treated with sodium hydrogen sulfide (0.29 g) and heated at 60° C. for 18 hours. The solvent was removed in a vacuum and the residue stirred with 1 M hydrochloric acid (15 mL) for 1 hour. The resulting solid was filtered, rinsed with water and dried in a vacuum at room temperature to give 4-(4-mercapto-5-methyl-2,6-dioxo-3,6-dihydro-2H-pyrimidin-1-ylmethyl)- benzoic acid tert-butyl ester (0.475 g, 91% yield). ¹H-NMR (DMSO-d₆) δ10.99 (s), 7.76(d, 2H), 7.27 (d, 2H), 5.52 (s, 2H), 1.91, (s, 3H), 1.50 (s, 9H).

Step (C): 4-[4-(Biphenyl-3-ylmethylsufmanyl)-5-methyl2,6-dioxo-3,6-dihydro-2H-pyrimidin-1-ylmethyl]benzoic acid t-butyl ester

4-(4-mercapto-5-methyl-2,6-dioxo-3,6-dihydro-2H-pyrimidin-1-ylmethyl)-benzoic acid tert-butyl ester (0.100 g, 0.29 mmol)) was dissolved in dimethylformamide (5 mL). 1-Phenyl-3-bromomethylbenzene (0.075 g, 0.3 mmol) was added and the solution heated at 60° C. for 3 hours. The dimethylformamide was evaporated in a vacuum. The residue was purified by flash chromatography on silica gel eluted with hexane:ethyl acetate (2:1) to give 4-[4-(biphenyl-3-ylmethylsufmanyl)-5-methyl2,6-dioxo-3,6-dihydro-2H-pyrimidin-1-ylmethyl]benzoic acid t-butyl ester (0.075 g, 50% yield). MS (APCl+), m/z 515, 500, 459.

Step (D): 4-[4-(Biphenyl-3-ylmethylsulfanyl)-5-methyl-2,6-dioxo-3,6-dihydro-2H-pyrimidin-1-ylmethyl]-benzoic acid 4-[4-(Biphenyl-3-ylmethylsufmanyl)-5-methyl2,6-dioxo-3,6-dihydro-2H-pyrimidin-1-ylmethyl]benzoic acid t-butyl ester (0.075 g) was stirred for 2 hours with trifluoroacetic acid (5 mL). The acid was evaporated in a vacuum. The resulting white solid was triturated with diethyl ether to give 4-[4-(biphenyl-3-ylmethylsulfanyl)-5-methyl-2,6-dioxo-3,6-dihydro-2H-pyrimidin-1 -ylmethyl]-benzoic acid (0.028 g, 42% yield). ¹H-NMR (CDCl₃) δ9.98 (s, 1H), 7.95 (d, 2H), 7.37-7.46 (m, 5H), 7.20-7.32 (m, 5H), 6.95 (d, 1H), 5.22 (s, 2H), 3.69 (s, 2H), 2.07 (s, 3H).

The preparation of inverse amide and ester allosteric inhibitors of MMP-13 is exemplified below in Preparation 5.

### PREPARATION 5

4-{7-[2-(4-Methoxy-phenyl)-acetylamino]-1-oxo-1H-isoquinolin-2-ylmethyl}-benzoic acid

Step (1) Preparation of 4-(7-amino-1-oxo-1H-isoquinolin-2-ylmethyl)benzoic acid tert-butyl ester

Into a sealed reactor was placed 4-(7-bromo-1-oxo-1H-isoquinolin-2-ylmethyl)benzoic acid tert-butyl ester (6.40 g, 15.4 mmol), copper bronze (0.1 g), and liquid NH₃ (80 mL). The reactor was heated to 70° C. for 62 hours, cooled to room temperature, filtered through Celite, and washed with tetrahydrofuran (“THF”). The filtrate was evaporated, the residue dissolved in EtOAc, and evaporated onto silica gel. The silica gel eluted on a 3.5×18cm silica gel column with ethyl acetate/hexanes 2:1. Evaporation of the appropriate fractions afforded a solid that was triturated with ether, collected, and dried to give 3.69 g (68.2%) of 4-(7-amino-1-oxo-1H-isoquinolin-2-ylmethyl)benzoic acid tert-butyl ester. ¹H-NMR (CDCl₃); 7.94-7.91 (d, 2H), 7.68-7.67 (d, 1H), 7.34-7.31 (m, 3H), 7.03-7.00 (d, 1H), 6.84-6.82 (d, 1H), 6.41-6.39 (d, 1H), 5.23 (s, 2H), 1.56 (s, 9H) MS:M⁺+1=351.1 Da

Step (2): Preparation of 4-{7-[2-(4-methoxyphenyl)acetylamino]-1-oxo-1H-isoquinolin-2-ylmethyl}benzoic acid tert-butyl ester

A solution of 4-methoxyphenylacetic acid (0.20 g, 1.20 mmol), EDAC.HCl (0.23 g, 1.20 mmol), and 1-hydroxybenzotriazole (“HOBT”, 0.16 g, 1.20 mmol) in dimethylformamide (“DMF”, 5 mL) was stirred at room temperature for 30 minutes. To this was added 4-(7-amino-1-oxo-1H-isoquinolin-2-ylmethyl)benzoic acid tert-butyl ester (0.30 g, 0.86 mmol, Step (1)) and the reaction mixture heated to 100° C. for 2 days. The reaction was cooled to room temperature, treated with water (2 mL), saturated aqueous NaHCO₃ (2 mL), then water (2 mL), and the mixture stirred at room temperature for 1 hour. The precipitated solid was collected by filtration, washed with water and dried. The solid was then triturated with hot hexanes/EtOAc 1:1, cooled to room temperature, and collected. Washing with hexanes/EtOAc and drying afforded 0.27 g (62.1%) of 4-{7-[2-(4-methoxyphenyl)acetylamino]-1-oxo-1H-isoquinolin-2-ylmethyl}benzoic acid tert-butyl ester.

¹H-NMR (DMSO); 10.39 (s, 1H), 8.47 (s, 1H), 7.93-7.92 (d, 1H), 7.84-7.82 (d, 2H), 7.57-7.59 (d, 1H), 7.44-7.42 (d, 1H), 7.37-7.35 (d, 2H), 7.25-7.24 (d, 2H), 6.89-6.86 (d, 2H), 6.60-6.59 (d, 1H), 5.23 (s, 2H), 3.70 (s, 3H), 3.57 (s, 2H), 1.50 (s, 9H). MS: M⁺+1=499.2 Da

Step (3): Preparation of 4-{7-[2-(4-methoxyphenyl)acetylamino]-1-oxo-1H-isoquinolin-2-ylmethyl}benzoic acid

The product of Step (2), namely 4-{7-[2-(4-methoxyphenyl)acetylamino-1-oxo-1H-isoquinolin-2-ylmethyl}benzoic acid tert-butyl ester, (0.18 g, 0.36 mmol), was treated with trifluoroacetic acid (“TFA”, 6 mL), then stirred at room temperature for 50 minutes. The TFA was evaporated, and the resulting solid triturated with hexanes/ethyl acetate (1:1), collected by filtration, washed with water, then hexanes/ethyl acetate (1:1). Drying afforded 0.14 g (89.5%) of 4-{7-[2-(4-methoxyphenyl)acetylamino]-1-oxo-1H-isoquinolin-2-ylmethyl}benzoic acid.

¹H-NMR (DMSO); 12.88 (s, 1H), 10.36 (s, 1H), 8.47 (s, 1H), 7.93-7.86 (m, 3H), 7.60-7.59 (d, 1H), 7.46-7.43 (d, 1H), 7.36-7.34 (2H), 7.26-7.23 (d, 2H), 6.87-6.85 (d, 2H), 6.61-6.59 (d, 1H), 5.23 (s, 2H), 3.70 (s, 3H), 3.56 (s, 2H) MS: M⁺+1 =443.1 Da

Any alpha-2-delta receptor ligand named above is readily available, either commercially, or by synthetic methodology, well known to those skilled in the art of organic chemistry. Preparations are described in the above-referenced patent documents.

Biological Activities:

A compound that is an allosteric inhibitor of MMP-13 may be readily identified by one of ordinary skill in the pharmaceutical or medical arts by assaying a test compound for inhibition of MMP-13 as described below in Biological Methods 1 or 2, and for allosteric inhibition of MMP-13 by assaying the test compound for inhibition of MMP-13 in the presence of an inhibitor to the catalytic zinc of MMP-13 as described below in Biological Methods 3 or 4.

It should be appreciated that for the purposes of the present invention, determining whether or not a test compound is an alpha-2-delta receptor ligand may be carried out by measuring the ability of the test compound to displace tritiated gabapentin from an alpha-2-delta receptor subtype-I or subtype-2 by conventional means. For example, the determination may be carried out with pig alpha-2-delta receptor 1 according to Biological Method 5 below.

Further, an invention combination having an anti-inflammatory, an analgesic, anti-arthritic, or a cartilage damage inhibiting effect, or any combination of these effects, may be readily identified by one of ordinary skill in the pharmaceutical or medical arts by assaying a test combination in any number of well known assays for determining the combination's effects on cartilage damage, arthritis, inflammation, or pain. These assays include in vitro assays that utilize cartilage samples and in vivo assays in whole animals that measure cartilage degradation, inhibition of inflammation, or pain alleviation.

For example with regard to assaying cartilage damage in vitro, an amount of the test combination or control vehicle may be administered with a cartilage damaging agent to cartilage, and the cartilage damage inhibiting effects in both tests studied by gross examination or histopathologic examination of the cartilage, or by measurement of biological markers of cartilage damage such as, for example, proteoglycan content or hydroxyproline content.

Further, in vivo assays to assay cartilage damage may be performed as follows: an amount of the test combination or control vehicle may be administered with a cartilage damaging agent to an animal, and the effects of the test combination being assayed on cartilage in the animal may be evaluated by gross examination or histopathologic examination of the cartilage, by observation of the effects in an acute model on functional limitations of the affected joint that result from cartilage damage, or by measurement of biological markers of cartilage damage such as, for example, proteoglycan content or hydroxyproline content.

Several methods of identifying a test combination with cartilage damage inhibiting properties are described below. The amount to be administered in an assay to identify the test combination is dependent upon the particular assay employed, but in any event is not higher than the well-known maximum amount of a active substance that the particular assay can effectively accommodate.

Similarly, a test combination having pain-alleviating properties may be identified using any one of a number of in vivo animal models of pain.

Still similarly, a test combination having anti-inflammatory properties may be identified using any one of a number of in vivo animal models of inflammation. For example, for an example of inflammation models, see U.S. Pat. No. 6, 329,429, which is incorporated herein by reference.

Still similarly, a test combination having anti-arthritic properties may be identified using any one of a number of in vivo animal models of arthritis. For example, for an example of arthritis models, see also U.S. Pat. No. 6, 329,429.

The allosteric inhibitors of MMP-13 identified herein have been shown to be potent and selective inhibitors of MMP-13 catalytic domain versus other MMP enzymes. To determine their inhibitory profiles, the allosteric inhibitors of MMP-13 were evaluated in standard assays for their ability to inhibit the catalytic activity of various MMP enzymes. The assays used to evaluate the MMP biological activity of the active compounds of this invention combination are well-known and routinely used by those skilled in the study of MMP inhibitors and their use to treat clinical conditions.

The assays measure the amount by which a test compound reduces the hydrolysis of a thiopeptolide substrate catalyzed by a matrix metalloproteinase enzyme. Such assays are described in detail by Ye et al., in Biochemistry, 1992;31(45):11231-11235, which is incorporated herein by reference. One such assay is described below in Biological Method 1.

Some of the particular methods described below use the catalytic domain of the MMP-13 enzyme, namely matrix metalloproteinase-13 catalytic domain (“MMP-13CD”), rather than the corresponding full-length enzyme, MMP-13. It has been shown previously by Ye Qi-Zhuang, Hupe D., and Johnson L. (Current Medicinal Chemistry, 1996;3:407-418) that inhibitor activity against a catalytic domain of an MMP is predictive of the inhibitor activity against the respective full-length MMP enzyme.

### BIOLOGICAL METHOD 1

Thiopeptolide substrates show virtually no decomposition or hydrolysis at or below neutral pH in the absence of a matrix metalloproteinase enzyme. A typical thiopeptolide substrate commonly utilized for assays is Ac-Pro-Leu-Gly-thioester-Leu-Leu-Gly-OEt. A 100 μL assay mixture will contain 50 mM of N-2-hydroxyethylpiperazine-N′-2-ethanesulfonic acid buffer (“HEPES,” pH 7.0), 10 mM CaCl ₂, 100 μM thiopeptolide substrate, and 1 mM 5,5′-dithio-bis-(2-nitro-benzoic acid) (DTNB). The thiopeptolide substrate concentration may be varied, for example from 10 to 800 μM to obtain Kₘ and K_{cat} values. The change in absorbance at 405 nm is monitored on a Thermo Max microplate reader (molecular Devices, Menlo Park, Calif.) at room temperature (22° C.). The calculation of the amount of hydrolysis of the thiopeptolide substrate is based on E₄₁₂=13600 M⁻¹ cm⁻¹ for the DTNB-derived product 3-carboxy-4-nitrothiophenoxide. Assays are carried out with and without matrix metalloproteinase inhibitor compounds, and the amount of hydrolysis is compared for a determination of inhibitory activity of the test compounds.

Test compounds were evaluated at various concentrations in order to determine their respective IC₅₀ values, the micromolar concentration of compound required to cause a 50% inhibition of catalytic activity of the respective enzyme.

It should be appreciated that the assay buffer used with MMP-3CD was 50 mM N-morpholinoethane sulfonate (“MES”) at pH 6.0 rather than the HEPES buffer at pH 7.0 described above.

The test described above for the inhibition of MMP-13 was also adapted and used to determine the ability of the allosteric inhibitors of MMP-13 to inhibit the matrix metalloproteases MMP-1, MMP-2, MMP-3, MMP-7, MMP-8, MMP-9, MMP-12, MMP-14, and/or MMP-17. The results obtained show that the allosteric inhibitors of MMP-13 generally have IC₅₀ values for MMP-13 which are about 100 times lower than the IC₅₀ values for the same compounds with respect to the other matrix metalloproteases tested.

### BIOLOGICAL METHOD 2

Some representative allosteric inhibitors of MMP-13 have been evaluated for their ability to inhibit MMP-13. Inhibitor activity versus other MMPs with the compounds may be determined using, for example, MMP-1FL, which refers to full length interstitial collagenase; MMP-2FL, which refers to full length Gelatinase A; MMP-3CD, which refers to the catalytic domain of stromelysin; MMP-7FL, which refers to full length matrilysin; MMP-9FL, which refers to full length Gelatinase B; MMP-13CD, which refers to the catalytic domain of collagenase 3; and MMP-14CD, which refers to the catalytic domain of MMP-14. Test compounds can be evaluated at various concentrations in order to determine their respective IC₅₀ values, the micromolar concentration of compound required to cause a 50% inhibition of the hydrolytic activity of the respective enzyme.

The results of the above assays with other MMPs have established that the allosteric inhibitors of MMP-13 identified above are potent inhibitors of MMP-13 enzymes, and are especially useful due to their selective inhibition of MMP-13. Because of this potent and selective inhibitory activity, the allosteric inhibitors of MMP-13 identified above are especially useful, in the invention combination to treat diseases mediated by MMP-13.

Allosteric inhibitors of MMP-13 may be readily identified by assaying a test compound for inhibition of MMP-13 according to the methods described below in Biological Methods 3 and 4.

### BIOLOGICAL METHOD 3

Fluorigenic peptide-1 substrate based assay for identifying allosteric inhibitors of MMP-13CD:

Final assay conditions:

50 mM HEPES buffer (pH 7.0)

10 mM CaCl₂

10 μM fluorigenic peptide-1 (“FP1”) substrate

0 or 15 mM acetohydroxamic acid (AcNHOH)=1 K_{d}

2% DMSO (with or without inhibitor test compound)

0.5 nM MMP-13CD enzyme

Stock solutions:

1) 10× assay buffer: 500 mM HEPES buffer (pH 7.0) plus 100 mM CaCl₂

2) 10 mM FP1 substrate: (Mca)-Pro-Leu-Gly-Leu-(Dnp)-Dpa-Ala-Arg—NH₂ (Bachem, M-1895; “A novel coumarin-labeled peptide for sensitive continuous assays of the matrix metalloproteinases,” Knight C. G., Willenbrock F., and Murphy, G., FEBS Lett., 1992;296:263-266). Prepared 10 mM stock by dissolving 5 mg FP1 in 0.457 mL DMSO.

3) 3 M AcNHOH: Prepared by adding 4 mL H₂O and 1 mL 10× assay buffer to 2.25 g AcNHOH (Aldrich 15,903-4). Adjusted pH to 7.0 with NaOH. Diluted volume to 10 mL with H₂O. Final solution contained 3 M AcNHOH, 50 mM HEPES buffer (pH 7.0), and 10 mM CaCl₂.

4) AcNHOH dilution buffer: 50 mM HEPES buffer (pH 7.0) plus 10 mM CaCl₂

5) MMP-13CD enzyme: Stock concentration=250 nM.

6) Enzyme dilution buffer: 50 mM HEPES buffer (pH 7.0), 10 mM CaCl₂, and 0.005% BRIJ 35 detergent (Calbiochem 203728; Protein Grade, 10%)

Procedure (for one 96-well microplate):

A. Prepared assay mixture:

1100 μL 10× assay buffer
11 μL 10 mM FP1
55 μL 3 M AcNHOH or 55 μL AcNHOH dilution buffer

8500 μL H₂O

B. Diluted MMP-13CD to 5 nM working stock:

22 μL MMP-13CD (250 nM)

1078 μL enzyme dilution buffer

C. Ran kinetic assay:

1. Dispensed 2 μL inhibitor test sample (in 100% DMSO) into well.

2. Added 88 μL assay mixture and mixed well, avoiding bubbles.

3. Initiated reactions with 10 μL of 5 nM MMP-13CD; mixed well, avoiding bubbles.

4. Immediately measured the kinetics of the reactions at room temperature.
Fluorimeter: Fmax Fluorescence Microplate Reader & SOFIMAX PRO Version 1.1 software (Molecular Devices Corporation; Sunnyvale, Calif. 94089).

Protocol menu:

| | |
|---|---|
| excitation: 320 nm | emission: 405 nm |
| run time: 15 min | interval: 29 sec |
| RFU min: −10 | RFU max: 200 |
| Vₘₐₓ points: 32/32 | |

D. Compared % of control activity and/or IC₅₀ with inhibitor test compound ±AcNHOH.

Hydrolysis of the fluorigenic peptide-1 substrate, [(Mca)Pro-Leu-Gly-Leu-Dpa-Ala-Arg—NH₂; Bachem, catalog number M-1895], wherein “Mca” is (7-methoxy-coumarin-4-yl)acetyl and “Dpa” is (3-[2,4-dinitrophenyl]-L-2,3-diaminopropionyl), was used to screen for MMP-13 catalytic domain (CD) inhibitors. (Dpa may also be abbreviated as “Dnp”.) Reactions (100 μL) contained 0.05 M Hepes buffer (pH 7), 0.01 M calcium chloride, 0.005% polyoxyethylene (23) lauryl ether (“Brij 35”), 0 or 15 mM acetohydroxamic acid, 10 μM FP1, and 0.1 mM to 0.5 nM inhibitor in DMSO (2% final).

After recombinant human MMP-13CD (0.5 nM final) was added to initiate the reaction, the initial velocity of FP1 hydrolysis was determined by monitoring the increase in fluorescence at 405 nm (upon excitation at 320 nm) continuously for up to 30 minutes on a microplate reader at room temperature. Alternatively, an endpoint read can also be used to determine reaction velocity provided the initial fluorescence of the solution, as recorded before addition of enzyme, is subtracted from the final fluorescence of the reaction mixture. The inhibitor was assayed at different concentration values, such as, for example, 100 μM, 10 μM, 1 μM, 100 nM, 10 nM, and 1 nM. Then the inhibitor concentration was plotted on the X-axis against the percentage of control activity observed for inhibited experiments versus uninhibited experiments (i.e., (velocity with inhibitor) divided by (velocity without inhibitor)×100) on the Y-axis to determine IC₅₀ values. This determination was done for experiments done in the presence, and experiments done in the absence, of acetohydroxamic acid. Data were fit to the equation: percent control activity=100/[1+(([I]/IC₅₀)^{slope})], where [I] is the inhibitor concentration, IC₅₀ is the concentration of inhibitor where the reaction rate is 50% inhibited relative to the control, and slope is the slope of the IC₅₀ curve at the curve's inflection point, using nonlinear least-squares curve-fitting equation regression.

Results may be expressed as an IC₅₀ Ratio (+/−) ratio, which means a ratio of the IC₅₀ of the inhibitor with MMP-13 and an inhibitor to the catalytic zinc of MMP-13, divided by the IC₅₀ of the inhibitor with MMP-13 without the inhibitor to the catalytic zinc of MMP-13. Allosteric inhibitors of MMP-13 have an IC₅₀ Ratio (+/−) ratio of less than 1, and are synergistic with the inhibitor to the catalytic zinc of MMP-13 such as, for example, AcNHOH. Compounds which are not allosteric inhibitors of MMP-13 will be inactive in the assay or will have an IC₅₀ Ratio (+/−) of greater than 1, unless otherwise indicated. Results can be confirmed by kinetics experiments that are well known in the biochemical art.

### BIOLOGICAL METHOD 4

Fluorigenic peptide-1 based assay for identifying allosteric inhibitors of matrix metalloproteinase-13 catalytic domain (“MMP-13CD”):

In a manner similar to Biological Method 3, an assay is run wherein 1,10-phenanthroline is substituted for acetohydroxamic acid to identify allosteric inhibitors of MMP-13CD.

Compounds that are ligands to an alpha-2-delta receptor may be identified according to the method described below in Biological Method 5.

### BIOLOGICAL METHOD 5

[³H]Gabapentin Scintillant Proximity A2DR1 and A2DR2 Binding Assays (“A2DR1 SPA” and “A2DR2 SPA”, respectively)

Step (1): Preparation of A2DR1 or A2DR2 protein

Recombinant HEK 293 cells expressing pig A2DR1 and A2DR2 subunits were grown under normal cell culture conditions (RPMI-1640 media with 10% FBS, 200μg G418, and 1% penicillin/streptomycin lt 37° C. with 5% CO₂) until reaching confluency in T-75 flasks, at which time they were harvested. The harvested cells were suspended in ice-cold SmM Tris/5mM ethylenediaminetetraacetic acid (“EDTA”) buffer, pH 7.4 (“TE buffer”) containing phenylmethylsulfonyl fluoride (“PMSF”) (0.1 mM) and Roche Complete Protease Inhibitor Cocktail, and allowed to sit on ice for 30 minutes. The cells were broken by sonication using 20 bursts, 40-50 cycles, and then centrifuged at 3000× g for 10 minutes. The resulting supernatant was transferred to a new tube and centrifuged at 50,000× g for 30 minutes. The resulting pellet was resuspended in 10 mM HEPES buffer, pH 7.4, homogenized, and stored at −80° C. The A2DR1 or A2DR2 membrane protein concentration was determined by the Pierce BCA method using bovine serum albumin (“BSA”) as the standard.

Step (2): Scintillant Proximity Assay (SPA).

The [³H]gabapentin SPA binding assay was performed in Costar 3632 96-well, clear bottom assay plates using Wheatgerm agglutinin beads (Amersham Pharmacia Biotech). Pig A2DR1 or A2DR2 membranes (10-20 μg protein per well) prepared above in Step (1) and SPA beads (0.5 mg per well) were mixed with 30 nM [³H]gabapentin (52 Ci/mmol; Amersham Pharmacia Biotech) in 10 mM HEPES/10 mM MgSO₄ assay buffer, pH 7.4 using KOH. The final well volume was 200 μL and non-specific binding was determined in the presence of 10 μM unlabeled pregabalin. The final mixture containing A2DR1 or A2DR2 membrane protein incubated with SPA beads, test compounds, and [³]gabapentin was incubated at room temperature for 15-24 hours, and the plates were then counted on a Wallace Trilux 1450 Microbeta scintillation counter.

Step (3): Determination of IC₅₀ values

Curve fitting and IC₅₀ values were calculated using a four-parameter, non-linear regression equation from GraphPad Prism 3.0 software, while Kᵢ values were determined using the equation of Chang and Prussoff.

Alternatively in Biological Method 5, test compound may be assayed at a single concentration, for example 10 μM, to preliminarily determine the presence or absence of a predetermined threshold level of binding activity.

An alpha-2-delta ligand, or a homolog, stereoisomer, or regioisomer thereof, an allosteric MMP-13 inhibitor, or a combination or pharmaceutical composition of this invention, may be assayed for cartilage damage inhibiting or osteoarthritic pain alleviating activities according to the method described below in Biological Method 6.

It should be appreciated that in the below studies, a dose in mg/kg means the weight in milligrams of test compound per weight of test animal body weight in kilograms.

### BIOLOGICAL METHOD 6

Monosodium lodoacetate-induced Osteoarthritis in Rat Model of Joint cartilage damage (“MIA Rat”):

One end result of the induction of osteoarthritis in this model, as determined by histologic analysis, is the development of an osteoarthritic condition within the affected joint, as characterized by the loss of Toluidine blue staining and formation of osteophytes. Associated with the histologic changes is a concentration-dependent degradation of joint cartilage, as evidenced by affects on hind-paw weight distribution of the limb containing the affected joint, the presence of increased amounts of proteoglycan or hydroxyproline in the joint upon biochemical analysis, or histopathological analysis of the osteoarthritic lesions.

The hind-paw weight distribution effects that would be expected to be observed for an invention combination or pharmaceutical composition result from the invention combination or pharmaceutical composition's ability to directly inhibit damage to cartilage or relieve joint pain.

Generally, In the MIA Rat model on Day 0, the hind-paw weight differential between the right arthritic joint and the left healthy joint of male Wistar rats (150 g) are determined with an incapacitance tester, model 2KG (Linton Instrumentation, Norfolk, United Kingdom). The incapacitance tester has a chamber on top with an outwardly sloping front wall that supports a rat's front limbs, and two weight sensing pads, one for each hind paw, that facilitates this determination. Rats are then anesthetized with 5% volume/volume (“v/v”) isoflurane gas until knocked down and maintained with 2% v/v isoflurane. The right, hind leg knee joint of each rat is injected with 1.0 mg of mono-iodoacetate (“MIA”) through the infrapatellar ligament. Injection of MIA into the joint results in the inhibition of glycolysis and eventual death of surrounding chondrocytes. Isoflurane administration is discontinued, and the rats become fully conscious after about 5 minutes. The rats are further administered either an invention combination, pharmaceutical composition, or vehicle (in the instant case, water) daily for 14 days or 28 days.

The invention combination is typically administered at a dose of 30 mg of per kilogram of rat per day (30 mg/kg/day, for example comprising 15 mg/kglday of an allosteric inhibitor of MMP-13, or a pharmaceutically acceptable salt thereof, and 15 mg/kg/day of an alpha-2-delta ligand, or a pharmaceutically acceptable salt thereof, administered separately in sequence or admixed and administered simultaneously), but may be administered at other doses such as, for example, 10 mg/kg/day, 60 mg/kg/day, 90-mg/kg/day, or 100 mg/kg/day according to the requirements of the invention combination being studied. It is well within the level of ordinary skill in the pharmaceutical arts to determine a proper dosage of an invention combination in this model.

Administration of an invention combination or pharmaceutical composition in this model is optionally by oral administration or by intravenous administration via an osmotic pump. After 7 and 14 days for a two-week study, or 7, 14, and 28 days for a four-week study, the hind-paw weight distribution is again determined. Typically, the animals administered vehicle alone place greater weight on their unaffected left hind paw than on their right hind paw, while animals administered an invention combination or pharmaceutical composition show a more normal (i.e., more like a healthy animal) weight distribution between their hind paws. This change in weight distribution is proportional to the degree of joint cartilage damage and joint pain. Percent inhibition of a change in hind paw joint function is calculated as the percent change in hind-paw weight distribution for treated animals versus control animals. For example, for a two week study,

Percent inhibition of a change in hind paw joint function

$$= \{ 1 - [ \frac{( Δ {W}_{G} )}{( Δ {W}_{C} )} ] \} \times 100$$

wherein: ΔW_{c} is the hind-paw weight differential between the healthy left limb and the arthritic limb of the control animal administered vehicle alone, as measured on Day 14; and

ΔW_{G} is the hind-paw weight differential between the healthy left limb and the arthritic limb of the animal administered an invention combination or pharmaceutical composition as measured on Day 14.

The hind paw weight bearing differentials are expressed in grams and calculated as follows: (the average weight in grams placed on the control limb minus the average weight in grams placed on the contralateral test limb for each of the inducement or treatment animals) minus (the average weight in grams placed on the control limb minus the average weight in grams placed on the contralateral test limb for each of the control animals). All results are ideally statistically analyzed by comparing the average results for an induction or treatment group with the average result for its control group at the same time point using analysis of covariance (“ANCOVA”) followed by the Hoehberg's procedure and those with statistical significance have ρ<0.05 unless otherwise noted.

It should be appreciated that it is not necessary to use ANCOVA followed by Hochberg's procedure to statistically analyze data in a method of this invention. Alternative statistical analyses are known that may be used such as ANCOVA without Hochberg's procedure, analysis of variance (“ANOVA”) with Hochberg's procedure, ANOVA without Hochberg's procedure, t-test with Hochberg's procedure, and t-test without Hochberg's procedure.

In order to measure biochemical or histopathological end points in the MIA Rat model for determining inhibition of cartilage damage, some of the animals in the above study may be sacrificed, and the amounts of free proteoglycan in both the osteoarthritic right knee joint and the contralateral left knee joint may be determined by biochemical analysis. The amount of free proteoglycan in the contralateral left knee joint provides a baseline value for the amount of free proteoglycan in a healthy joint. The amount of proteoglycan in the osteoarthritic right knee joint in animals administered an invention combination or pharmaceutical composition and the amount of proteoglycan in the osteoarthritic right knee joint in animals administered vehicle alone, are independently compared to the amount of proteoglycan in the contralateral left knee joint. The amounts of proteoglycan lost in the osteoarthritic right knee joints are expressed as percent loss of proteoglycan compared to the contralateral left knee joint control. The percent inhibition of proteoglycan loss, may be calculated as, for example,{[(proteoglycan loss from joint (%) with vehicle)—(proteoglycan loss from joint with invention combination or pharmaceutical composition]÷ (proteoglycan loss from joint (%) with vehicle)}×100.

If any invention combination or pharmaceutical composition were to be tested in a conventional animal (mammal) model of tumor invasion, inflammation, pain, cartilage damage, and the like such as carrageenan induced foot edema (inflammation and inflammatory pain), monosodium iodoacetate induced osteoarthritis in rat (osteoarthritis, osteoarthritic pain), or experimental osteoarthritis in rabbit, the results would be expected to show that the invention combination or pharmaceutical composition would reduce the size of the lesion on the tibial plateaus, and perhaps the damage in the tibia or on the femoral condyles, as well as show pain alleviating effects if measured. In conclusion, these results would show that an invention combination or pharmaceutical composition would have significant inhibition effects on inflammation, pain, and/or the damage to cartilage, bone, and/or extracellular matrix.

The foregoing studies would establish that an invention combination or pharmaceutical composition is effective for the inhibition of cartilage damage and inflammation and/or alleviating pain, and thus useful for the treatment of breast cancer, osteoarthritis or rheumatoid arthritis in human, and other mammalian disorders described above. Such a treatment offers a distinct advantage over existing treatments that only modify pain or inflammation or and other secondary symptoms. The effectiveness of an invention combination in this model would indicate that the invention combination would have clinically useful effects in preventing and/or treating damage to cartilage, bone, extracellular matrix, joint pain, bone cancer, breast cancer, arthritis, and/or inflammation.

In one aspect of this invention, administration according to the invention method of an invention combination to a mammal to treat the diseases listed above is accomplished by administering the combination, or a salt thereof, in a pharmaceutical dosage form.

Pharmaceutical Dosage Forms:

The active ingredients of the invention combination can be prepared and administered according to the invention method in a wide variety of oral and parenteral pharmaceutical dosage forms. Thus, the active ingredients can be administered by injection, that is, intravenously, intramuscularly, intracutaneously, subcutaneously, intraduodenally, or intraperitoneally. Also, the drugs can be administered by inhalation, for example, intranasally. Additionally, the active ingredients can be administered transdermally. It will be obvious to those skilled in the art that the following dosage forms may optionally comprise as the active components both an alpha-2-delta receptor ligand, or a pharmaceutically acceptable salt thereof, and an allosteric inhibitor of MMP-13, or a pharmaceutically acceptable salt thereof, or the active components each in a separate, and optionally different dosage form and amount. The active compounds generally are present in a concentration of about 5% to about 95% by weight of the formulation.

For preparing pharmaceutical compositions from an alpha-2-delta receptor ligand, or a pharmaceutically acceptable salt thereof, and the allosteric inhibitors of MMP-13, or a pharmaceutically acceptable salt thereof, (i.e., the active components) pharmaceutically acceptable carriers can be either solid or liquid. In one aspect of this invention, solid form preparations are utilized. Solid form preparations include powders, tablets, pills, capsules, cachets, suppositories, and dispersible granules. A solid carrier can be one or more substances that may also act as diluents, flavoring agents, solubilizers, lubricants, suspending agents, binders, preservatives, tablet disintegrating agents, or an encapsulating material.

In powders, the carrier is a finely divided solid that is in a mixture with the finely divided active component. Powders suitable for intravenous administration or administration by injection may be lyophilized.

In tablets, the active component is mixed with the carrier having the necessary binding properties in suitable proportions and compacted in the shape and size desired.

In one aspect of this invention, powders and tablets contain from about 5% to about 70%, total, of the active component. Suitable carriers are magnesium carbonate, magnesium stearate, talc, sugar, lactose, pectin, dextrin, starch, gelatin, tragacanth, methylcellulose, sodium carboxymethylcelulose, a low melting wax, cocoa butter, and the like. The term “preparation” is intended to include the formulation of the active component with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

In one aspect of this invention, the pharmaceutical preparation is in unit dosage form. In such form, the preparation is subdivided into unit doses containing an appropriate quantity of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

In one aspect of this invention, the quantity of active component in a unit dose preparation may be varied or adjusted from 0.01 to 1000 mg or from 1 to 500 mg according to the particular application and the potency of the active components. The composition can, if desired, also contain other compatible therapeutic agents as described above.

In therapeutic use as agents to treat the above-listed diseases, the active components are administered at doses that are effective for treating at least one symptom when a single disease or disorder is being treated or at doses that are effective for treating at least one symptom of each of two diseases or disorders being co-treated according to the invention method.

The initial dosage of about 1 mg/kg to about 100 mg/kg daily of an active component will be effective. In one aspect of this invention, a daily dose range of about 25 mg/kg to about 75 mg/kg of an active component is utilized. The dosages, however, may be varied depending upon the requirements of the patient, the severity of the condition being treated, and the particular drugs being employed in the invention combination. Determination of the proper dosage for a particular situation is within the skill of the art as described above. Typical dosages will be from about 0.1 mg/kg to about 500 mg/kg, and ideally about 25 mg/kg to about 250 mg/kg, such that it will be an amount that is effective to treat the particular disease or disorder being treated.

In another aspect of this invention, a composition for dogs comprises an ingestible liquid peroral dosage form selected from the group consisting of a solution, suspension, emulsion, inverse emulsion, elixir, extract, tincture and concentrate, optionally to be added to the drinking water of the dog being treated. Any of these liquid dosage forms, when formulated in accordance with methods well known in the art, can either be administered directly to the dog being treated, or may be added to the drinking water of the dog being treated. The concentrate liquid form, on the other hand, is formulated to be added first to a given amount of water, from which an aliquot amount may be withdrawn for administration directly to the dog or addition to the drinking water of the dog.

In another aspect of this invention, a composition provides delayed-, sustained- and/or controlled-release of active ingredients. Such pharmaceutical compositions include all such dosage forms which produce≧40% inhibition of a symptom such as pain or swelling, or a pathology such as cartilage degradation, and result in a plasma concentration of at least one, and, in another aspect of this invention, at least two, active component(s) of at least 3 fold the active component's ED₄₀ for at least 2 hours; in another aspect of this invention, for at least 4 hours; in another aspect of this invention, for at least 8 hours; in another aspect of this invention, for at least 12 hours; in another aspect of this invention, still for at least 16 hours; in another aspect of this invention, still for at least 20 hours; and in another aspect of this invention, for at least 24 hours. In another aspect of this invention, there is included within the above-described dosage forms those which produce≧40% inhibition of the symptom or pathology, and result in a plasma concentration of at least one, and, in another aspect of this invention, at least two active component(s) of at least 5 fold the active component's ED₄₀ for at least 2 hours, in another aspect of this invention, for at least 2 hours, in another aspect of this invention, for at least 8 hours, in another aspect of this invention, for at least 12 hours, in another aspect of this invention, for at least 20 hours and in another aspect of this invention, for at least 24 hours. In another aspect of this invention, there is included the above-described dosage forms which produce≧50% inhibition of the symptom or pathology, and result in a plasma concentration of at least one, and, in another aspect of this invention, at least two, active component(s) of at least 5 fold the active component's ED₄₀ for at least 2 hours, in another aspect of this invention, for at least 4 hours, in another aspect of this invention, for at least 8 hours, in another aspect of this invention, for at least 12 hours, in another aspect of this invention, for at least 20 hours and in another aspect of this invention, for at least 24 hours.

The above Formulations 1 to 3 illustrate the invention pharmaceutical compositions and dosage forms wherein the allosteric inhibitor of MMP-13, or a pharmaceutically acceptable salt thereof, and an alpha-2-delta receptor ligand, or a pharmaceutically acceptable salt thereof, are formulated together or separately, each independently as described. The formulations are representative only, and are not to be construed as limiting the invention in any respect.

Still further, it should be appreciated that the invention methods comprising administering an invention combination to a mammal to treat diseases or disorders listed above may be used to treat different diseases simultaneously. For example, administration of an alpha-2-delta receptor ligand, or a pharmaceutically acceptable salt thereof, in accordance with the invention combination may be carried out as described above to treat inflammation, arthritic pain, breast cancer pain, neuropathic pain, fibromyalgia, or epilepsy, while an allosteric inhibitor of MMP-13, or a pharmaceutically acceptable salt thereof, may be administered to treat OA, OA pain, breast cancer, rheumatoid arthritis, or inhibit cartilage damage.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

All patent documents referenced above, including the U.S. patents, U.S. patent applications, U.S. patent application publications, EP patents, EP patent application publications, and PCT WO publications referenced above, are hereby incorporated herein by reference in their entireties for all purposes.

Having described this invention in clear detail, certain aspects of this invention are hereupon claimed. The number of claims herein has been limited for practical reasons. Applicants reserve their right to add claims to additional aspects of this invention in the future.

1. A combination, comprising an allosteric inhibitor of MMP-13, or a pharmaceutically acceptable salt thereof, and a ligand to an alpha-2-delta receptor, or a pharmaceutically acceptable salt thereof, wherein the allosteric inhibitor of MMP-13 is a compound of Formula IB 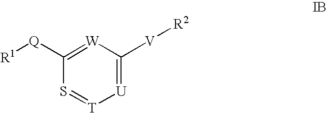
or a pharmaceutically acceptable salt thereof, wherein: each of R₁ and R2 is independently selected from:
Phenyl-(C₁-C₆ alkylenyl); and
Substituted phenyl-(C₁-C₆ alkylenyl);
5-, 6-, 9-, and 10-membered heteroaryl-(C₁-C₆ alkylenyl); and
Substituted 5-, 6-, 9-, and 10-membered heteroaryl-(C₁-C₆ alkylenyl); S, T, U, and W each are C-R⁴; or One of S, T, U, and W is N and the other three of S, T, U, and W are C-R⁴; Each R⁴independently is selected from: H, CH₃, or OCH₃; V is a 5-membered heteroarylenyl which is: 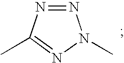
Q is selected from: N(H)C(O); Each “substituted” group contains from 1 to 4 substituents, each
independently on a carbon or nitrogen atom, independently selected from: C₁-C₆ alkyl, C₂-C₆ alkenyl, C₂-C₆ alkynyl, C₃-C₆ cycloalkyl, C₃-C₆ cycloalkylmethyl, Phenyl, Phenylmethyl, 3- to 6-membered heterocycloalkyl, 3- to 6-membered heterocycloalkylmethyl, cyano, CF₃, (C₁—C₆ alkyl)-OC(O), HOCH₂, (C₁—C₆ alkyl)—OCH₂, H₂NCH₂, (C₁—C₆ alkyl)—NH(H)CH₂, (C₁—C₆ alkyl)₂—NCH₂, N(H)₂C(O), (C₁—C₆ alkyl)—NH(H)C(O), (C₁—C₆ alkyl)₂—NC(O), N(H)₂C(O)N(H), (C₁—C₆ alkyl)—NH(H)C(O)N(H), N(H)₂C(O)N(C₁—C₆ alkyl), (C₁—C₆ alkyl)—NH(H)C(O)N(C₁—C₆ alkyl), (C₁—C₆ alkyl)₂—NC(O)N(H), (C₁—C₆ alkyl)₂—NC(O)N(C₁—C₆ alkyl), N(H)₂C(O)O, (C₁—C₆ alkyl)—NH(H)C(O)O, (C₁—C₆ alkyl)₂—NC(O)O, HO, (C₁—C₆ alkyl)—O, CF₃O, CF₂(H)O, CF(H)₂O, H₂N, (C₁—C₆ alkyl)—NH(H), (C₁—C₆ alkyl)₂—N, O₂N, (C₁—C₆ alkyl)—S, (C₁—C₆ alkyl)—S(O), (C₁—C₆ alkyl)—S(O)₂, (C₁—C₆ alkyl)₂—NS(O)₂, (C₁—C₆ alkyl)—S(O)₂—NH(H)—C(O)—(C₁—C8 alkylenyl)ₘ, and (C₁—C₆ alkyl)—C(O)—NH(H)—S(O)₂—(C₁—C₈ alkylenyl)ₘ;
wherein each substituent on a carbon atom may further be independently selected from: Halo, HO₂C, and OCH₂O, wherein each O is bonded to adjacent carbon atoms to form a 5-membered ring;
wherein 2 substituents may be taken together with a carbon atom to which they are both bonded to form the group C═O;
wherein each m independently is an integer of 0 or 1;
wherein each 5-membered heteroarylenyl independently is a 5-membered ring containing carbon atoms and from 1 to 4 heteroatoms selected from 1 O, 1 S, 1 NH, 1 N(C₁-C₆ alkyl), and 4 N, wherein the O and S atoms are not both present, and wherein the heteroarylenyl may optionally be unsubstituted or substituted with 1 substituent selected from fluoro, methyl, hydroxy, trifluoromethyl, cyano, and acetyl;
wherein each heterocycloalkyl is a ring that contains carbon atoms and 1 or 2 heteroatoms independently selected from 2 O, 1 S, 1 S(O), 1 S(O)₂, 1 N, 2 N(H), and 2 N(C₁-C₆ alkyl), and wherein when two O atoms or one O atom and one S atom are present, the two O atoms or one O atom and one S atom are not bonded to each other, and wherein the ring is saturated or optionally contains one carbon-carbon or carbon-nitrogen double bond;
wherein each 5-membered heteroaryl contains carbon atoms and from 1 to 4 heteroatoms independently selected from 1 O, 1 S, 1 N(H), 1 N(C₁-C₆ alkyl), and 4 N, and each 6-membered heteroaryl contains carbon atoms and 1 or 2 heteroatoms independently selected from N, N(H), and N(C₁-C₆ alkyl), and 5- and 6-membered heteroaryl are monocyclic rings; and 9- and 10-membered heteroaryl are 6,5-fused and 6,6-fused bicyclic rings, respectively, wherein at least 1 of the 2 fused rings of a bicyclic ring is aromatic, and wherein when the 0 and S atoms both are present, the O and S atoms are not bonded to each other;
wherein with any (C₁-C₆ alkyl)₂-N group, the C₁-C₆ alkyl groups may be optionally taken together with the nitrogen atom to which they are attached to form a 5- or 6-membered heterocycloalkyl; and
wherein each group and each substituent recited above is independently selected.

2. The combination according to claim 1, wherein the alpha-2-delta receptor ligand is pregabalin.

3. The combination according to claim 1, wherein the alpha-2-delta receptor ligand is gabapentin.

4. The combination according to claim 1, wherein the alpha-2-delta receptor ligand is selected from:
3-(1-aminomethyl-cyclohexylmethyl)-4H-[1,2,4]oxadiazol-5-one;
C-[1-(1H-tetrazol-5-ylmethyl)-cycloheptyl]-methylamine;
(3S,5R)-3-aminomethyl-5-methyl-octanoic acid;
(S,S)-(1-aminomethyl-3,4-dimethyl-cyclopentyl)-acetic acid;
[(lR,5R,6S)-6-(aminomethyl)bicyclo[3.2.0]hept-6-yl]acetic acid;
(1α,3α, 5α)(3-amino-methyl-bicyclo[3.2.0]hept-3-yl)-acetic acid;
(3S,5R)-3-amino-5-methyl-nonanoic acid;
(3S,5R)-3-amino-5-methyl-octanoic acid;
(3S,5R)-3-amino-5-methyl-heptanoic acid; or
a pharmaceutically acceptable salt thereof.

5. The combination according to claim 1, wherein the allosteric inhibitor of MMP-13 is a compound of Formula IB, or a pharmaceutically acceptable salt thereof, selected from the group consisting of:
4-({3-[2-(4-Methoxy-benzyl)-2H-tetrazol-5-yl]-benzoylamino}-methyl)-benzoic acid methyl ester;
4-({3-[2-(4-Methoxy-benzyl)-2H-tetrazol-5-yl]-benzoylamino}-methyl)-benzoic acid;
4-({3-[2-(3-Methoxy-benzyl)-2H-tetrazol-5-yl]-benzoylamino}-methyl)-benzoic acid methyl ester;
4-({3-[2-(3-Methoxy-benzyl)-2H-tetrazol-5-yl]-benzoylamino}-methyl)-benzoic acid;
3-[2-(4-Methoxy-benzyl)-2H-tetrazol-5-yl]-N-(4-morpholin-4-ylmethyl-benzyl)-benzamide;
3-[2-(4-Methoxy-benzyl)-2H-tetrazol-5-yl]-N-(3-trifluoromethyl-benzyl)-benzamide;
N-Benzyl-3-[2-(4-methoxy-benzyl)-2H-tetrazol-5-yl]-benzamide;
3-[2-(4-Methoxy-benzyl)-2H-tetrazol-5-yl]-N-(2-trifluoromethyl-benzyl)-benzamide; and
N-(4-Methoxy-benzyl)-3-[2-(4-methoxy-benzyl)-2H-tetrazol-5-yl]-benzamide;
or a pharmaceutically acceptable salt thereof.

6. The combination according to claim 1, wherein the allosteric inhibitor of MMP-13 is a compound of Formula IB, or a pharmaceutically acceptable salt thereof, selected from the group consisting of:
4-({3-[2-(4-Fluoro-benzyl)-2H-tetrazol-5-yl]-benzoylamino}-methyl)-benzoic acid methyl ester;
4-({3-[2-(4-Fluoro-benzyl)-2H-tetrazol-5-yl]-benzoylamino}-methyl)-benzoic acid;
4-({3-[2-(3-Fluoro-benzyl)-2H-tetrazol-5-yl]-benzoylamino}-methyl)-benzoic acid methyl ester;
4-({3-[2-(3-Fluoro-benzyl)-2H-tetrazol-5-yl]-benzoylamino}-methyl)-benzoic acid;
N-(3—Chloro-4-fluoro-benzyl)-3-[2-(4-methoxy-benzyl)-2H-tetrazol-5-yl]-benzamide;
N-(2,3-Difluoro-benzyl)-3-[2-(4-methoxy-benzyl)-2H-tetrazol-5-yl]-benzamide; and
N-(4-Fluoro-benzyl)-2-methoxy-5-[2-(4-methoxy-benzyl)-2H-tetrazol-5-yl]-benzamide; or
a pharmaceutically acceptable salt thereof.

7. The combination according to claim 1, wherein the allosteric inhibitor of MMP-13 is a compound of Formula IB, or a pharmaceutically acceptable salt thereof, selected from the group consisting of:
N-Benzyl-3-[2-(4-cyano-benzyl)-2H-tetrazol-5-yl]-benzamide;
4-{[3-(2-Thiazol-2-ylmethyl-2H-tetrazol-5-yl)-benzoylamino]-methyl}-benzoic acid methyl ester;
4-{[3-(2-But-2-enyl-2H-tetrazol-5-yl)-benzoylamino]-methyl}benzoic acid methyl ester;
N-Benzyl-3-(2-but-2-enyl-2H-tetrazol-5-yl)-benzamide;
3-(2-But-2-enyl-2H-tetrazol-5-yl)-N-(3-methoxy-benzyl)-benzamide;
3-[2-(4-Methoxy-benzyl)-2H-tetrazol-5-yl]-N-thiazol-2-ylmethyl-benzamide;
N-2,1,3-Benzothiadiazol-5-ylmethyl-3-[2-(4-methoxy-benzyl)-2H-tetrazol-5-yl]-benzamide;
3-[2-(4-Methoxy-benzyl)-2H-tetrazol-5-yl]-N-(2-methoxy-pyridin-4-ylmethyl)-benzamide;
3-[2-(4-Methoxy-benzyl)-2H-tetrazol-5-yl]-N-pyridin-4-ylmethyl-benzamide;
N-1,3-Benzodioxol-5-ylmethyl-3-[2-(4-methoxy-benzyl)-2H-tetrazol-5-yl]-benzamide; and
N-Furan-2-ylmethyl-3-[2-(4-methoxy-benzyl)-2H-tetrazol-5-yl]-benzamide; or
a pharmaceutically acceptable salt thereof.

8. The combination according to claim 1, wherein the allosteric inhibitor of MMP-13 is a compound of Formula IB, or a pharmaceutically acceptable salt thereof, selected from the group consisting of:
4-(5-{3-[(Pyridin-4-ylmethyl)-carbamoyl]-phenyl}-tetrazol-2-ylmethyl)-benzoic acid;
4-(5-{3-[(Pyridin-3-ylmethyl)-carbamoyl]-phenyl}-tetrazol-2-ylmethyl)-benzoic acid;
4-(5-{3-[(2-Methoxy-pyridin-4-ylmethyl)-carbamoyl]-phenyl}-tetrazol-2-ylmethyl)-benzoic acid;
3-[2-(4-Methoxy-benzyl)-2H-tetrazol-5-yl]-N-(2-pyridin-4-yl-ethyl)-benzamide;
N-Isopropyl-3-[2-(4-methoxy-benzyl)-2H-tetrazol-5-yl]-benzamide;
3-[2-(4-Methoxy-benzyl)-2H-tetrazol-5-yl]-N-( 1 -phenyl-ethyl)-benzamide;
4-(5-{3-[(Methyl-pyridin-3-ylmethyl)-carbamoyl]-phenyl}-tetrazol-2-ylmethyl)-benzoic acid;
4-({2-Methoxy-5-[2-(4-methoxy-benzyl)-2H-tetrazol-5-yl]-benzoylamino}-methyl)-benzoic acid;
4-({2-Methoxy-5-[2-(4-methoxy-benzyl)-2H-tetrazol-5-yl]-benzoylamino}-methyl)-benzoic acid; and
2-Methoxy-5-[2-(4-methoxy-benzyl)-2H-tetrazol-5-yl]-N-(4-trifluoromethyl-benzyl)-benzamide;
or a pharmaceutically acceptable salt thereof.

9. A pharmaceutical composition, comprising the combination according to claim 1, and a pharmaceutically acceptable carrier, diluent, or excipient.

10. A method of treating a disease or disorder selected from: joint cartilage damage, joint inflammation, joint stiffness, impaired joint function, joint pain, osteoarthritis, and rheumatoid arthritis in a mammal suffering therefrom, comprising administering to the mammal a therapeutically effective, sufficiently nontoxic amount of the pharmaceutical composition according to claim 9.
